(19) United States
(12) Patent Application Publication
(10) Pub. No.: US 2025/0129140 A1
(43) Pub. Date: Apr. 24, 2025

(54) ANTI-HCMV ANTIBODIES AND ANTIGEN-BINDING FRAGMENTS THEREOF

(71) Applicant: ICAHN SCHOOL OF MEDICINE AT MOUNT SINAI, New York, NY (US)

(72) Inventors: Domenico Tortorella, New York, NY (US); James A. Duty, New York, NY (US); Andrea Parsons, New York, NY (US); Thomas Moran, New York, NY (US); Thomas Kraus, New York, NY (US)

(21) Appl. No.: 18/688,491

(22) PCT Filed: Sep. 2, 2022
(86) PCT No.: PCT/US2022/075879

§ 371 Date: Mar. 1, 2024

Related U.S. Application Data
(60) Provisional application No. 63/240,489, filed on Sep. 3, 2021.

(51) Int. Cl. C07K 16/08; A61K 39/00; A61P 31/22

(52) U.S. Cl. CPC C07K 16/089; A61P 31/22; A61K 2039/505; C07K 2317/24; C07K 2317/33; C07K 2317/76; C07K 2317/92

(57) ABSTRACT

Provided herein are recombinant antibodies and antigen-binding fragments thereof useful for binding to HCMV. Also provided are methods of using the disclosed anti-HCMV antibodies and antigen-binding fragments thereof for treating and preventing HCMV infections in a subject in need thereof.

### FIELD OF THE DISCLOSURE

The present disclosure relates to broadly neutralizing anti-human cytomegalovirus (anti-HCMV) antibodies, vaccines, and kits, as well as methods of use, including diagnostic and therapeutic methods.

### BACKGROUND

Human cytomegalovirus (HCMV) is a β-herpesvirus with an estimated global seroprevalence rate of 83%. In immune competent hosts, the majority of CMV primary infections are asymptomatic. However, the control of CMV in immune-compromised individuals is of great importance as it can be spread during organ transplantation and across the placenta from mother to fetus, as is the case with congenital CMV infection.

While CMV vaccine efforts are underway, strategies to prevent transmission include the use of antivirals and CMV hyperimmune globulin (CytoGam®). Despite positive findings for the use of CytoGam® as a prophylactic treatment to reduce CMV infections, overall efficacy and side effects severely limit its use.

Accordingly, safe and effective anti-CMV therapeutics are urgently needed.

### SUMMARY

Provided herein are antibodies and antigen-binding fragments thereof that bind to HCMV. Also provided are therapeutic compositions of such antibodies and antigen-binding fragments thereof, as well as methods of using these antibodies and antigen-binding fragments thereof.

In one aspect, provided is an antibody or antigen-binding fragment thereof which binds to human cytomelagovirus (HCMV), the antibody or antigen-binding fragment thereof comprising a heavy chain variable region and a light chain variable region, wherein each of the heavy chain and the light chain variable regions comprises a CDR1, CDR2, and CDR3, and wherein:
(a) the sequence of CDR1H comprises SEQ ID NO:33;
   the sequence of CDR2H comprises SEQ ID NO:34;
   the sequence of CDR3H comprises SEQ ID NO:35;
   the sequence of CDR1L comprises SEQ ID NO:36;
   the sequence of CDR2L comprises sequence AAS; and
   the sequence of CDR3L comprises SEQ ID NO:37;
(b) the sequence of CDR1H comprises SEQ ID NO:40;
   the sequence of CDR2H comprises SEQ ID NO:41;
   the sequence of CDR3H comprises SEQ ID NO:42;
   the sequence of CDR1L comprises SEQ ID NO:43;
   the sequence of CDR2L comprises sequence AAS; and
   the sequence of CDR3L comprises SEQ ID NO:44;
(c) the sequence of CDR1H comprises SEQ ID NO:47;
   the sequence of CDR2H comprises SEQ ID NO:48;
   the sequence of CDR3H comprises SEQ ID NO:49;
   the sequence of CDR1L comprises SEQ ID NO:50;
   the sequence of CDR2L comprises sequence GAS; and
   the sequence of CDR3L comprises SEQ ID NO:51; or
(d) the sequence of CDR1H comprises SEQ ID NO:54;
   the sequence of CDR2H comprises SEQ ID NO:55;
   the sequence of CDR3H comprises SEQ ID NO:56;
   the sequence of CDR1L comprises SEQ ID NO:57;
   the sequence of CDR2L comprises sequence AAS; and
   the sequence of CDR3L comprises SEQ ID NO:58.

In one embodiment, provided is an anti-HCMV antibody or antigen-binding fragment thereof, wherein:
(a) the sequence of the heavy chain variable region comprises a sequence that is at least 90% identical to SEQ ID NO:38 and wherein the sequence of the light chain variable region comprises a sequence that is at least 90% identical to SEQ ID NO:39;
(b) the sequence of the heavy chain variable region comprises a sequence that is at least 90% identical to SEQ ID NO:45 and wherein the sequence of the light chain variable region comprises a sequence that is at least 90% identical to SEQ ID NO:46;
(c) the sequence of the heavy chain variable region comprises a sequence that is at least 90% identical to SEQ ID NO:52 and wherein the sequence of the light chain variable region comprises a sequence that is at least 90% identical to SEQ ID NO:53; or
(d) the sequence of the heavy chain variable region comprises a sequence that is at least 90% identical to SEQ ID NO:59 and wherein the sequence of the light chain variable region comprises a sequence that is at least 90% identical to SEQ ID NO:60.

In one embodiment, provided is an anti-HCMV antibody or antigen-binding fragment thereof, wherein:
(a) the sequence of the heavy chain variable region comprises a sequence that is at least 95% identical to SEQ ID NO:38 and wherein the sequence of the light chain variable region comprises a sequence that is at least 95% identical to SEQ ID NO:39;
(b) the sequence of the heavy chain variable region comprises a sequence that is at least 95% identical to SEQ ID NO:45 and wherein the sequence of the light chain variable region comprises a sequence that is at least 95% identical to SEQ ID NO:46;
(c) the sequence of the heavy chain variable region comprises a sequence that is at least 95% identical to SEQ ID NO:52 and wherein the sequence of the light chain variable region comprises a sequence that is at least 95% identical to SEQ ID NO:53; or
(d) the sequence of the heavy chain variable region comprises a sequence that is at least 95% identical to SEQ ID NO:59 and wherein the sequence of the light chain variable region comprises a sequence that is at least 95% identical to SEQ ID NO:60.

In one embodiment, provided is an anti-HCMV antibody or antigen-binding fragment thereof, wherein:
(a) the sequence of the heavy chain variable region comprises SEQ ID NO:38 and the sequence of the light chain variable region comprises SEQ ID NO:39;
(b) the sequence of the heavy chain variable region comprises SEQ ID NO:45 and the sequence of the light chain variable region comprises SEQ ID NO:46;
(c) the sequence of the heavy chain variable region comprises SEQ ID NO:52 and the sequence of the light chain variable region comprises SEQ ID NO:53; or
(d) the sequence of the heavy chain variable region comprises SEQ ID NO:59 and the sequence of the light chain variable region comprises SEQ ID NO:60.

In one embodiment, the anti-HCMV antibody or antigen-binding fragment thereof is a chimeric antibody, a CDR-grafted antibody, or a humanized antibody or antigen-binding fragment thereof.

In one embodiment, the anti-HCMV antibody or antigen-binding fragment thereof is a monoclonal antibody or antigen-binding fragment thereof.

In one embodiment, the anti-HCMV antibody or antigen-binding fragment thereof is a multispecific or a bispecific antibody or antigen-binding fragment thereof.

In one embodiment, the anti-HCMV antibody or antigen-binding fragment thereof is an scFv, Fv, Fab′, Fab, F(ab′)₂, or diabody.

In one embodiment, the anti-HCMV antibody or antigen-binding fragment thereof has isotype IgG2.

In one embodiment, the anti-HCMV antibody or antigen-binding portion thereof is capable of broadly neutralizing an HCMV infection.

Provided herein is a nucleic acid encoding an anti-HCMV antibody or antigen-binding fragment thereof disclosed herein. In some embodiments, the nucleic acid is isolated. Provided herein is a vector comprising a nucleic acid disclosed herein.

Provided herein is a vector or set of vectors encoding an antibody or antigen-binding fragment thereof which binds to HCMV, the antibody or antigen-binding fragment thereof comprising a heavy chain variable region and a light chain variable region, wherein:
(a) the sequence encoding the heavy chain variable region comprises a sequence that is at least 80% identical to SEQ ID NO:7 and the sequence encoding the light chain variable region comprises a sequence that is at least 80% identical to SEQ ID NO:8;
(b) the sequence encoding the heavy chain variable region comprises a sequence that is at least 80% identical to SEQ ID NO:15 and the sequence encoding the light chain variable region comprises a sequence that is at least 80% identical to SEQ ID NO:16;
(c) the sequence encoding the heavy chain variable region comprises a sequence that is at least 80% identical to SEQ ID NO:23 and the sequence encoding the light chain variable region comprises a sequence that is at least 80% identical to SEQ ID NO:24; or
(d) the sequence encoding the heavy chain variable region comprises a sequence that is at least 80% identical to SEQ ID NO:31 and the sequence encoding the light chain variable region comprises a sequence that is at least 80% identical to SEQ ID NO:32.

Provided herein is a vector or set of vectors encoding an antibody or antigen-binding fragment thereof which binds to HCMV, the antibody or antigen-binding fragment thereof comprising a heavy chain variable region and a light chain variable region, wherein:
(a) the sequence encoding the heavy chain variable region comprises SEQ ID NO:7 and the sequence encoding the light chain variable region comprises SEQ ID NO:8;
(b) the sequence encoding the heavy chain variable region comprises SEQ ID NO: 15 and the sequence encoding the light chain variable region comprises SEQ ID NO:16;
(c) the sequence encoding the heavy chain variable region comprises SEQ ID NO:23 and the sequence encoding the light chain variable region comprises SEQ ID NO:24; or
(d) the sequence encoding the heavy chain variable region comprises SEQ ID NO:31 and the sequence encoding the light chain variable region comprises SEQ ID NO:32.

Provided herein is a cell comprising a vector disclosed herein or a vector or set of vectors disclosed herein. In some embodiments, the cell is a bacterial cell, a yeast cell, or an isolated mammalian cell. The cell may be isolated.

Provided herein is a pharmaceutical composition comprising an anti-HCMV antibody or antigen-binding fragment thereof disclosed herein and a pharmaceutically acceptable carrier or excipient.

Provided herein is a T-cell comprising a chimeric antigen receptor comprising the CDRs of an anti-HCMV antibody or antigen-binding fragment thereof disclosed herein.

In some embodiments, the anti-HCMV antibody or antigen-binding fragment thereof conjugated to one or more of a cytotoxin, a fluorescent label, and an imaging agent.

Provided herein is a kit for detecting the presence of HCMV, or an antigenic fragment of HCMV thereof, in a sample comprising: (i) an anti-HCMV antibody or antigen-binding portion thereof disclosed herein, and (ii) a buffer. In one embodiment, the anti-HCMV antibody or antigen-binding portion thereof is bound to a substrate. In one embodiment, the anti-HCMV antibody or antigen-binding portion thereof is detectably labeled. In one embodiment, the kit further comprising a secondary antibody that specifically binds to the antibody or antigen-binding portion thereof. In one embodiment, the secondary antibody is an anti-IgG antibody. In one embodiment, the secondary antibody is detectably labeled.

In one aspect, provided is a method of making an antibody or antigen-binding fragment thereof which binds to HCMV, the method comprising:
(i) providing a cell comprising one or more nucleic acid molecules encoding an anti-HCMV antibody or antigen-binding fragment thereof disclosed herein;
(ii) expressing in the cell at least one of a heavy variable chain, a light variable chain, or combinations thereof; and
(iii) collecting the anti-HCMV antibody or antigen-binding fragment thereof.

In one aspect, provided is a method of detecting the presence of HCMV, or an antigenic fragment thereof, in a sample comprising:
(i) obtaining a sample containing, or suspecting of containing, HCMV, or an antigenic fragment thereof;
(ii) contacting the sample with an anti-HCMV antibody or antigen-binding fragment thereof of any disclosed herein; and
(iii) detecting the presence of specific binding of the anti-HCMV antibody or antigen-binding fragment thereof to HCMV, or an antigenic fragment thereof.

In some embodiments, the method further comprises quantifying the amount of HCMV, or antigenic fragments thereof, present in the sample. In some embodiments, the sample is an environmental sample. In some embodiments, the sample is a biological sample.

In one aspect, provided is a method of treating an HCMV infection in a subject in need thereof comprising, the method comprising administering to the subject an anti-HCMV antibody or antigen-binding fragment disclosed herein. In one embodiment, the method further comprises administering to the individual at least one additional anti-HCMV antibody or antigen-binding portion thereof. In one embodiment, the at least one additional anti-HCMV antibody, or antigen-binding portion thereof, is an anti-HCMV antibody or antigen-binding fragment disclosed herein. In one embodiment, the method comprises administering to the individual at least one additional antiviral composition. In some embodiments, the at least one additional antiviral composition is selected from the group consisting of ganciclovir, valganciclovir, foscarnet, cidofovir, and combinations thereof.

In one aspect, provided is a method of preventing an HCMV infection in a subject comprising administering to the individual an anti-HCMV antibody or antigen-binding fragment thereof of disclosed herein.

In one aspect, provided is a method of diagnosing a subject as having an HCMV infection comprising:
(i) identifying a subject;
(ii) obtaining from the subject a biological sample containing HCMV or an antigenic fragment thereof;
(iii) contacting the sample with an anti-HCMV antibody or antigen-binding fragment thereof disclosed herein;
(iv) detecting the presence of specific binding of the anti-HCMV antibody or antigen-binding fragment thereof to HCMV, or an antigenic fragment thereof; and
(v) diagnosing the subject as having an HCMV infection.

In one aspect, provided is a method of inhibiting binding of HCMV glycoprotein gH and/or glycoprotein gL to a cellular surface protein, the method comprising contacting gH and/or gL with an anti-HCMV antibody or antigen-binding fragment thereof disclosed herein. In some embodiments, the cellular surface protein is selected from the group consisting of Nectin 1, EphA2, Nrp2, PDGFRalpha.

Provided herein is an anti-HCMV antibody or antigen-binding fragment thereof disclosed herein for use in medicine.

Provided herein is an anti-HCMV antibody or antigen-binding fragment thereof disclosed herein for use in treating or preventing an HCMV infection.

Provided herein is the use of an anti-HCMV antibody or antigen-binding fragment thereof disclosed herein in the manufacture of a medicament for use in treating or preventing an HCMV infection.

### BRIEF DESCRIPTION OF THE DRAWINGS

FIG. 1A, FIG. 1B. FIG. 1C, and FIG. 1D illustrate that vaccination elicits the generation of neutralizing antibodies identified through hybridoma screening. FIG. 1A and FIG. 1B. Mice were vaccinated with HCMV purified virus (CMV strains are indicated) or plasmid (labeled “DNA”) encoding the gH/gL dimer. Serum was isolated at Day 146. Neutralization capacity of mouse sera was tested in both epithelial (ARPE-19) (FIG. 1A) and fibroblast (MRC5) (FIG. 1B) cells using reporter virus AD169R. Percent infection was quantified using green fluorescent protein (GFP) and normalized to virus incubated in the presence of normal mouse serum (NMS). FIG. 1C and FIG. 1D. Hybridoma supernatants from fusion 1 were screened for neutralization AD169R in ARPE-19 (FIG. 1C) and MRC5 (FIG. 1D) cells and relative percent infection was normalized to virus alone.

FIG. 2A and FIG. 2B show that the isolated anti-HCMV antibodies disclosed herein are broadly neutralizing. FIG. 2A. Increasing concentrations of monoclonal antibodies (0.016-50 μg/mL) were incubated with AD169R (MOI 0.2) to assess neutralizing capacity across three cell lines. Percent infection was quantified using GFP expression at 18 hpi and normalized based on infection with virus alone (no antibody). FIG. 2B. Monoclonal antibodies were used to neutralize four HCMV strains (MOI 0.2) in fibroblast cells using a 5-fold serial dilution starting at 50 μg/mL. Relative percent infection at 18 hpi was determined using IE1-1 as a readout for infection.

FIG. 3A, FIG. 3B, FIG. 3C, FIG. 3D, FIG. 3E, and FIG. 3F show that the isolated anti-HCMV antibodies disclosed herein significantly reduce plaque formation in ARPE-19 cells. The relative number of plaques (>10,000 μm²) on Day 7 post infection in both ARPE-19 (FIG. 3A, FIG. 3B. and FIG. 3C) and NHDF (FIG. 3D, FIG. 3E. and FIG. 3F) are shown for both concentrations and for each antibody using M2E10 (α-IAV) and CytoGam® as negative and positive controls per plate.

FIG. 4A and FIG. 4B illustrate the isolated anti-HCMV antibodies disclosed herein bind gH. FIG. 4A. ARPE-19 cells were infected with AD169R and collected 6 dpi for immunostaining to characterize antibody binding by flow cytometry using 2 μg/mL. FIG. 4B. U373 astrocytoma cell lines were transduced to constitutively express HCMV glycoproteins gB, gH/gL, gH/gL/gO or gH/gL/UL128. Antibodies (2 μg/mL) were used to assess binding to cell surface (top panel) or intracellularly using permeabilized cells (bottom panel).

FIG. 5 illustrates that the isolated anti-HCMV antibodies disclosed herein bind two distinct regions of gH. Competition assays were performed with U373 gH/gL cells incubated with increasing concentrations of unlabeled antibody (5-0.1 μg/mL) indicated along x-axis and a constant amount (0.5 μg/mL) of labeled 5C3 (top), 6E1 (middle), and 10F8 (bottom). The relative MFI of AF647 positive cells is depicted after being normalized to the average MFI when labeled antibody was incubated with irrelevant influenza antibody PY102.

FIG. 6A and FIG. 6B illustrate that α-gH antibodies bind two distinct regions of gH. FIG. 6A. Representative plot for binding of 9A12 to overlapping peptide libraries spanning 7, 10 and 13 aa in length. FIG. 6B. Diagram of full-length gH outlining location of each alanine mutation tested for epitope mapping. pcDNA gH_{AAAA} was expressed with gL in BHK cells and binding was quantified by immunofluorescence 2 dpt.

FIG. 7 shows that α-gH antibodies can be used in combination with Ganciclovir. Relative number of plaques present 12 dpi in ARPE-19 cells infected with AD169R. Infection was performed in the presence of antibody at three different concentrations +/−ganciclovir (2.5 μM). On day 12 post infection GFP images were taken and the plaque number was calculated relative to virus alone controls on each plate. M2E10 and CytoGam® were used as negative and positive controls respectively. Each condition was done in triplicate and 10,000 μm² was used as a cutoff for plaque area.

FIG. 8A, FIG. 8B, FIG. 8C, and FIG. 8D show that fully human anti-HCMV antibodies maintain their broadly neutralizing capacity. Increasing concentrations of monoclonal antibodies (0.016-50 μg/mL) were incubated with AD169R (MOI 0.2) to assess neutralizing capacity. Percent infection was quantified using GFP expression at 18 hpi and normalized based on infection with virus alone (no antibody). CMV hyperimmune globulin (CytoGam®) was used as a control (trace on the upper right in FIGS. 8A, 8B, 8C, and 8D). FIG. 8A. Fully human (hu, comprising human variable and human constant regions) and chimeric (ms, comprising human variable and murine constant regions) versions of 15G11. FIG. 8B. Fully human (hu, comprising human variable and human constant regions) and chimeric (ms, comprising human variable and murine constant regions) versions of 9A12. FIG. 8C. Fully human (hu, comprising human variable and human constant regions) and chimeric (ms, comprising human variable and murine constant regions) versions of 13G1. FIG. 8D. Fully human (hu, comprising human variable and human constant regions) and chimeric (ms, comprising human variable and murine constant regions) versions of 14E1.

FIG. 9 illustrates that fully human anti-CMV mAbs limit CMV infection and proliferation in a SCID animal. Severe combined immunodeficiency disease (SCID) mice implanted with CMV infected cells imbedded in gelfoam were injected intraperitoneally with isotype control, anti-gH mAbs 9A12, 13G1, and 15G11 and ganciclovir every three days for up to nine days. At day ten post-implantation, the cells were released from the gelfoam and DNA extracted from the cells was subjected to qPCR for the HCMV UL123 and β-actin genes. The relative CMV levels cells were determined from Ct values of qPCR analysis (in triplicate) from cells collected from three mice/treatment. Statistical tests were performed using ordinary one-way ANOVA with comparisons to isotype treated cells as a control and a Dunnett's post-test; **, p<0.01; ***, p<0.001; ****, p<0.0001.

### DETAILED DESCRIPTION

### Human Cytomegalovirus

Human cytomegalovirus is the largest β-herpesvirus with a linear dsDNA genome of 235 kb, which codes for >165 viral proteins and contains several miRNAs and ncRNAs. Like all herpes viruses, HCMV establishes a lifelong infection, maintaining a latent reservoir within the bone marrow. Viral shedding can occur after reactivation or after reinfection with a second strain of HCMV. The estimated global seroprevalence of HCMV is 83% with strong evidence for higher prevalence in lower socio-economic groups and women of childbearing age, the latter attributed to increased contact with young children. Infections in immune competent hosts are typically asymptomatic but can cause severe, life-threatening complications in individuals who are immune compromised or have immature immune systems. High-risk groups for HCMV include patients with immune disorders such as AIDS, transplant recipients and infants. Congenital HCMV infections occur when the mother has a reactivation or primary infection immediately before, during or after pregnancy and passes the virus to the infant. Congenital CMV infection is the leading cause of neurological damage in children and can be associated with severe birth defects including sensorineural hearing loss, microcephaly, and periventricular calcifications. Although multiple clinical trials have been conducted to identify a safe and effective therapy to reduce transmission and/or reduce disease severity in congenitally infected infants none have been approved, further emphasizing the importance of novel strategies to treat CMV.

Most of the current FDA-approved drugs for the treatment of CMV including ganciclovir, valganciclovir, foscarnet and cidofovir target viral replication by interfering with DNA polymerase with varying efficacy and dose-related cytotoxicity. The use of hyperimmune globulin (HIG) isolated from CMV seropositive donors has been approved as a prophylaxis of CMV disease with kidney and renal transplant patients but use in other designations such as AIDS patients, HSCT or congenital CMV infection has not been approved. The use of HIG requires high doses, has a short half-life and thus needs to be given frequently, requires IV administration, contains antibodies that are not specific to CMV, and has been shown to have significant lot to lot variability which impairs treatment efficacy.

Of the herpesviruses, HCMV harbors the most genes dedicated to evading the host immune response, including adaptive immunity, and represents a significant life-long burden of antigenic T-cell surveillance and immune dysfunction. Accordingly, the viral envelope of HCMV contains various protein complexes that enable wide viral tropism, utilizing multiple glycoprotein complexes to attach and fuse with host cell membranes including the membranes of fibroblasts, epithelial, endothelial, and myeloid cells.

Glycoproteins gB, gH, and gL comprise the core fusion machinery and exist in various protein complexes on the virion surface. Glycoprotein gB catalyzes membrane fusion during viral entry, and gH and gL likely serve as factors which activate gB to permit pH-independent fusion at the cellular membrane. In addition to the gH/gL heterodimer, gH and gL exist in the trimeric gH/gL/gO complex which is essential for viral entry into fibroblasts. The pentameric complex (PC), which consists of gH/gL and three additional proteins UL128, UL130 and UL131a, is required for viral entry into epithelial, endothelial, and myeloid cells where the virion enters through a low pH-dependent endocytosis mechanism. The glycoprotein complex gH/gL/gO (gH trimer) is required for infection of all cell types, while the gH/gL/UL128/130/131a (gH pentamer) complex imparts specificity in infecting epithelial, endothethial, and myeloid cells. Given that the amount of gH/gL/gO trimer on the viral surface correlates with levels of CMV entry in both epithelial and fibroblast cells, gH may contribute to viral entry primarily through activation of the fusion event, rather than serving a receptor-binding role. Without wishing to be bound by theory, anti-gH antibodies may function by interrupting a fusion-triggering signal to gB.

### Antibodies

The term “antibody” is used in the broadest sense and includes monoclonal antibodies (including full length or intact monoclonal antibodies), polyclonal antibodies, multivalent antibodies, multispecific antibodies (e.g., bispecific antibodies), antibody fragments, and antigen-binding portions thereof (e.g., paratopes, CDRs), so long as they exhibit the desired biological activity and specificity. The terms “antigen-binding portion” or “antigen-binding fragment” as used herein may refer to a region on an antibody that binds to its antigen.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). V_{H} refers to the variable domain of the heavy chain. V_{L} refers to the variable domain of the light chain. The amino acid positions assigned to CDRs and FRs may be defined according to Kabat or according to Chothia. The term “framework regions” (FR) refers to those variable domain residues other than the CDR residues.

As used herein, the term “Complementarity Determining Regions” (CDRs) refers to portions of an antibody variable domain that are (typically) involved in antigen-binding. Each variable domain typically has three CDR regions identified as CDR1, CDR2 and CDR3. Each CDR can comprise amino acid residues from a CDR as defined by e.g. Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1987, 1991)). Each CDR can also comprise amino acid residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain (Chothia & Lesk 196 J. Mol. Biol. 901 (1987)). In some instances, a CDR can include amino acids from both a CDR region defined according to Kabat and a hypervariable loop. The Kabat residue designations do not always correspond directly with the linear numbering of the amino acid residues (primary amino acid sequence). The actual linear amino acid sequence may contain fewer or additional amino acids than in the strict Kabat numbering corresponding to a shortening of, or insertion into, a structural component, whether framework or CDR, of the basic variable domain structure. The correct Kabat numbering of residues may be determined for a given antibody or antigen-binding fragment thereof by alignment of residues of homology in the sequence of the antibody or antigen-binding fragment thereof with a “standard” Kabat numbered sequence. Alternatively, a CDR can be defined according to the ImMunoGeneTics (IMGT) system (Lefranc, M. P. et al., Dev. Comp. Immunol., 27, 55-77 (2003)).

### Epitope

In one aspect, the antibodies or antigen-binding fragments disclosed herein bind to glycoprotein gH and/or glycoprotein gL. Without wishing to be bound by theory, antibodies that specifically bind to glycoprotein gH may be capable of broadly neutralizing HCMV because of the role glycoprotein gH plays in viral entry. Glycoprotein gH is part of a heterodimer, as well as a trimeric complex and a pentameric complex, and is required for viral entry, serving as a factor to permit cell surface fusion. Thus, while not wishing to be bound by theory, these anti-HCMV antibodies or antigen-binding portions thereof which target glycoprotein gH are capable of broadly inhibiting HCMV infection because they are blocking and/or disrupting viral entry pathways.

The full length of glycoprotein gH, generated by consensus sequence, is reproduced below:

(SEQ ID NO: 63)
MRPGLPFYLTVFAVYLLSHLPSQRYGADAASEALDPHAFHLLLNTYGRPIRFLRENT
TQCTYNSSLRNSTVVRENAISFNFFQSYNQYYVFHMPRCLFAGPLAEQFLNQVDLTE
TLERYQQRLNTYALVSKDLASYRSFSQQLKAQDSLGQQPTTVPPPIDLSIPHVWMPP
QTTPHDWKGSHTTSGLHRPHFNQTCILFDGHDLLFSTVTPCLHQGFYLMDELRYVKI
TLTEDFFVVTVSIDDDTPMLLIFGHLPRVLFKAPYQRDNFILRQTEKHELLVLVKKTQ
LNRHSYLKDSDFLDAALDENYLDLSALLRNSFHRYAVDVLKSGRCQMLDRRTVEM
AFAYALALFAAARQEEAGTEISIPRALDRQAALLQIQEFMITCLSQTPPRTTLLLYPTA
VDLAKRALWTPDQITDITSLVRLVYILSKQNQQHLIPQWALRQIADFALQLHKTHLA
SFLSAFARQELYLMGSLVHSMLVHTTERREIFIVETGLCSLAELSHFTQLLAHPHHEY
LSDLYTPCSSSGRRDHSLERLTRLFPDATVPATVPAALSILSTMQPSTLETFPDLFCLPL
GESFSALTVSEHVSYVVTNQYLIKGISYPVSTTVVGQSLIITQTDSQSKCELTRNMHTT
HSITAALNISLENCAFCQSALLEYDDTQGVINIMYMHDSDDVLFALDPYNEVVVSSP
RTHYLMLLKNGTVLEVTDVVVDATDSRLLMMSVYALSAIIGIYLLYRMLKTC.

The full length of glycoprotein gL, generated by consensus sequence, is reproduced below:

(SEQ ID NO: 64)
MCRRPDCGFSFSPGPVVLLWCCLLLPIVSSVAVSVAPTAAEKVPAECPE
LTRRCLLGEVFQGDKYESWLRPLVNVTRRDGPLSQLIRYRPVTPEAANS
VLLDDAFLDTLALLYNNPDQLRALLTLLSSDTAPRWMTVMRGYSECGDG
SPAVYTCVDDLCRGYDLTRLSYGRSIFTEVLGFELVPPSLFNVVVAIRN
EATRTNRAVRLPVSTAAAPEGITLFYGLYNAVKEFCLRHQLDPPLLRHL
DKYYAGLPPELKQTRVNLPAHSRYGPQAVDAR.

On one embodiment, the anti-HCMV antibody or antigen-binding fragment thereof is one of the antibodies disclosed in Table 1.

TABLE 1
Amino acid sequences of antibodies 13G1, 14E1, 15G11, and 9A12

| | 13G1 | 14E1 | 15G11 | 9A12 |
|---|---|---|---|---|
| Description | (SEQ ID NO) | (SEQ ID NO) | (SEQ ID NO) | (SEQ ID NO) |
| CDR1H | 33 | 40 | 47 | 54 |
| CDR2H | 34 | 41 | 48 | 55 |
| CDR3H | 35 | 42 | 49 | 56 |
| CDR1L | 36 | 43 | 50 | 57 |
| CDR2L | AAS | AAS | GAS | AAS |
| CDR3L | 37 | 44 | 51 | 58 |
| VH | 38 | 45 | 52 | 59 |
| VL | 39 | 46 | 53 | 60 |

In one embodiment, the anti-HCMV antibody or antigen-binding fragment thereof provided herein comprises six CDRs, wherein:
(a) the sequence of CDR1H comprises SEQ ID NO:33;
(b) the sequence of CDR2H comprises SEQ ID NO:34;
(c) the sequence of CDR3H comprises SEQ ID NO:35;
(d) the sequence of CDR1L comprises SEQ ID NO:36;
(e) the sequence of CDR2L comprises sequence AAS; and
(f) the sequence of CDR3L comprises SEQ ID NO:37.

In one embodiment, the anti-HCMV antibody or antigen-binding fragment thereof provided herein comprises six CDRs, wherein:
(a) the sequence of CDR1H comprises SEQ ID NO:40;
(b) the sequence of CDR2H comprises SEQ ID NO:41;
(c) the sequence of CDR3H comprises SEQ ID NO:42;
(d) the sequence of CDR1L comprises SEQ ID NO:43;
(e) the sequence of CDR2L comprises sequence AAS; and
(f) the sequence of CDR3L comprises SEQ ID NO:44.

In one embodiment, the anti-HCMV antibody or antigen-binding fragment thereof provided herein comprises six CDRs, wherein:
(a) the sequence of CDR1H comprises SEQ ID NO:47;
(b) the sequence of CDR2H comprises SEQ ID NO:48;
(c) the sequence of CDR3H comprises SEQ ID NO:49;
(d) the sequence of CDR1L comprises SEQ ID NO:50;
(e) the sequence of CDR2L comprises sequence GAS; and
(f) the sequence of CDR3L comprises SEQ ID NO:51.

In one embodiment, the anti-HCMV antibody or antigen-binding fragment thereof provided herein comprises six CDRs, wherein:
(a) the sequence of CDR1H comprises SEQ ID NO:54;
(b) the sequence of CDR2H comprises SEQ ID NO:55;
(c) the sequence of CDR3H comprises SEQ ID NO:56;
(d) the sequence of CDR1L comprises SEQ ID NO:57;
(e) the sequence of CDR2L comprises sequence AAS; and
(f) the sequence of CDR3L comprises SEQ ID NO:58.

Also provided herein are anti-HCMV antibodies or antigen-binding fragments thereof comprising variable heavy chain and variable light chain sequences or pairings thereof that comprise sequences that are similar, but not identical to, the variable heavy chain and variable light chains disclosed in SEQ ID NOs: 38, 39, 45, 46, 52, 53, 59, and 60 and pairings thereof.

In some embodiments, the anti-HCMV antibody or antigen-binding fragment thereof comprises a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NOs: 38, 45, 52, or 59.

In some embodiments, the anti-HCMV antibody or antigen-binding fragment thereof comprises a variable heavy chain amino acid sequence comprising any one of SEQ ID NOs: 38, 45, 52, or 59.

In some embodiments, the anti-HCMV antibody or antigen-binding fragment thereof comprises a light chain variable domain comprising a sequence that is at least 80%, at least 85%, at least 90%, at least 95%, at least 96%, at least 97%, at least 98%, or at least 99% identical to a light chain variable domain sequence of SEQ ID NOs: 39, 46, 53, or 60.

In some embodiments, the anti-HCMV antibody or antigen-binding fragment thereof comprises a variable light chain amino acid sequence comprising any one of SEQ ID NOs: 39, 46, 53, or 60.

In one embodiment, the anti-HCMV antibody or antigen-binding fragment thereof that comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NOs: 38, 45, 52, or 59; and/or
(ii) a light chain variable domain comprising a sequence that is at least 80%, at least 85%, at least 90%, at least 95%, at least 96%, at least 97%, at least 98%, or at least 99% identical to a light chain variable domain sequence of SEQ ID NOs: 39, 46, 53, or 60.

In one embodiment, the anti-HCMV antibody or antigen-binding fragment thereof that comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:38; and/or
(ii) a light chain variable domain comprising a sequence that is at least 80%, at least 85%, at least 90%, at least 95%, at least 96%, at least 97%, at least 98%, or at least 99% identical to a light chain variable domain sequence of SEQ ID NO:39.

In one embodiment, the anti-HCMV antibody or antigen-binding fragment thereof that comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:45; and/or
(ii) a light chain variable domain comprising a sequence that is at least 80%, at least 85%, at least 90%, at least 95%, at least 96%, at least 97%, at least 98%, or at least 99% identical to a light chain variable domain sequence of SEQ ID NO:46.

In one embodiment, the anti-HCMV antibody or antigen-binding fragment thereof that comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:52; and/or
(ii) a light chain variable domain comprising a sequence that is at least 80%, at least 85%, at least 90%, at least 95%, at least 96%, at least 97%, at least 98%, or at least 99% identical to a light chain variable domain sequence of SEQ ID NO:53.

In one embodiment, the anti-HCMV antibody or antigen-binding fragment thereof that comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:59; and/or
(ii) a light chain variable domain comprising a sequence that is at least 80%, at least 85%, at least 90%, at least 95%, at least 96%, at least 97%, at least 98%, or at least 99% identical to a light chain variable domain sequence of SEQ ID NO:60.

In one embodiment, the anti-HCMV antibody or antigen-binding fragment thereof that comprises
(i) a heavy chain variable domain comprising SEQ ID NOs: 38, 45, 52, or 59; and/or
(ii) a light chain variable domain comprising SEQ ID NOs: 39, 46, 53, or 60.

In one embodiment, the anti-HCMV antibody or antigen-binding fragment thereof that comprises
(i) a heavy chain variable domain comprising SEQ ID NO:38; and/or
(ii) a light chain variable domain comprising SEQ ID NO:39.

In one embodiment, the anti-HCMV antibody or antigen-binding fragment thereof that comprises
(i) a heavy chain variable domain comprising SEQ ID NO:45; and/or
(ii) a light chain variable domain comprising SEQ ID NO:46.

In one embodiment, the anti-HCMV antibody or antigen-binding fragment thereof that comprises
(i) a heavy chain variable domain comprising SEQ ID NO:52; and/or
(ii) a light chain variable domain comprising SEQ ID NO:53.

In one embodiment, the anti-HCMV antibody or antigen-binding fragment thereof that comprises
(i) a heavy chain variable domain comprising SEQ ID NO:59; and/or
(ii) a light chain variable domain comprising SEQ ID NO:60.

As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In one embodiment, provided is an anti-HCMV antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:38;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:39; and
(iii) six CDRs, wherein:
   a. the sequence of CDR1 of the heavy chain variable region comprises SEQ ID NO:33;
   b. the sequence of CDR2 of the heavy chain variable region comprises SEQ ID NO:34;
   c. the sequence of CDR3 of the heavy chain variable region comprises SEQ ID NO: 35;
   d. the sequence of CDR1 of the light chain variable region comprises SEQ ID NO:36;
   e. the sequence of CDR2 of the light chain variable region comprises sequence AAS; and
   f. the sequence of CDR3 of the light chain variable region comprises SEQ ID NO:37.

In one embodiment, provided is an anti-HCMV antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:45;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:46; and
(iii) six CDRs, wherein:
   a. the sequence of CDR1 of the heavy chain variable region comprises SEQ ID NO:40;
   b. the sequence of CDR2 of the heavy chain variable region comprises SEQ ID NO:41;
   c. the sequence of CDR3 of the heavy chain variable region comprises SEQ ID NO: 42;
   d. the sequence of CDR1 of the light chain variable region comprises SEQ ID NO:43;
   e. the sequence of CDR2 of the light chain variable region comprises sequence AAS; and
   f. the sequence of CDR3 of the light chain variable region comprises SEQ ID NO:44.

In one embodiment, provided is an anti-HCMV antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:52;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:53; and
(iii) six CDRs, wherein:
   a. the sequence of CDR1 of the heavy chain variable region comprises SEQ ID NO:47;
   b. the sequence of CDR2 of the heavy chain variable region comprises SEQ ID NO:48;
   c. the sequence of CDR3 of the heavy chain variable region comprises SEQ ID NO: 49;
   d. the sequence of CDR1 of the light chain variable region comprises SEQ ID NO:50;
   e. the sequence of CDR2 of the light chain variable region comprises sequence GAS; and
   f. the sequence of CDR3 of the light chain variable region comprises SEQ ID NO:51.

In one embodiment, provided is an anti-HCMV antibody or antigen-binding fragment thereof comprises
(i) a heavy chain variable domain comprising a sequence that is at least 80%, at least 85%, at least 90%, at least 95%, at least 96%, at least 97%, at least 98%, or at least 99% identical to a heavy chain variable domain sequence of SEQ ID NO:59;
(ii) a light chain variable domain comprising a sequence that is least 80%, at least 85%, at least 90%, at least 95%, at least 96%, at least 97%, at least 98%, or at least 99% identical to a heavy chain variable domain sequence of SEQ ID NO:60; and
(iii) six CDRs, wherein:
   a. the sequence of CDR1 of the heavy chain variable region comprises SEQ ID NO:54;
   b. the sequence of CDR2 of the heavy chain variable region comprises SEQ ID NO:55;
   c. the sequence of CDR3 of the heavy chain variable region comprises SEQ ID NO: 56;
   d. the sequence of CDR1 of the light chain variable region comprises SEQ ID NO:57;
   e. the sequence of CDR2 of the light chain variable region comprises sequence AAS; and
   f. the sequence of CDR3 of the light chain variable region comprises SEQ ID NO:58.

It will be evident that any of the frameworks described herein can be utilized in combination with any of the CDRs and CDR motifs described herein.

### Antibody Modifications

In some embodiments of the aspects described herein, amino acid sequence modification(s) of the antibodies or antigen-binding fragments thereof that bind to HCMV described herein are contemplated. Amino acid sequence variants of the antibody or antigen-binding fragment thereof are prepared by introducing appropriate nucleotide changes into the nucleic acid encoding the antibody or antigen-binding fragment thereof, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, provided that the final construct possesses the desired characteristics, e.g., binding specificity, and inhibition of biological activity.

One type of variant is a conservative amino acid substitution variant. These variants have at least one amino acid residue in the antibody or antigen-binding fragment thereof replaced by a different residue that has similar side chain properties. Amino acids can be grouped according to similarities in the properties of their side chains (see Lehninger, BIOCHEMISTRY (2nd ed., Worth Publishers, New York, 1975):
(1) non-polar: Ala (A), Val (V), Leu (L), Ile (I), Pro (P), Phe (F), Trp (W), Met (M);
(2) uncharged polar: Gly (G), Ser(S), Thr (T), Cys (C), Tyr (Y), Asn (N), Gln (Q);
(3) acidic: Asp (D), Glu (E);
(4) basic: Lys (K), Arg (R), His (H).
As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser(S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).
As such, a non-limiting example for a conservative amino acid substitution is one that replaces a hydrophobic amino acid with another hydrophobic amino acid.

In some embodiments, the CDRs of an anti-HCMV antibody or antigen-binding fragment disclosed herein has a conservative amino acid substitution.

Further contemplated are amino acid sequence insertions, which can include amino- and/or carboxyl-terminal fusions ranging in length from one residue to polypeptides containing a hundred or more residues, as well as intrasequence insertions of single or multiple amino acid residues. Examples of terminal insertions include an antibody or antigen-binding fragment thereof with an N-terminal methionyl residue or the antibody or antigen-binding fragment thereof fused to a cytotoxic polypeptide. Other insertional variants of the antibody or antigen-binding fragment thereof include the fusion to the N- or C-terminus of the antibody or antigen-binding fragment thereof to an enzyme or a polypeptide which increases the serum half-life of the antibody or antigen-binding fragment thereof, such as, for example, biotin.

Any cysteine residue not involved in maintaining the proper conformation of the antibodies or antigen-binding fragments thereof that bind to HCMV also can be substituted, for example with a serine or an alanine, to improve the oxidative stability of the molecule and prevent aberrant crosslinking.

Conversely, cysteine bond(s) can be added to the antibody or antigen-binding fragment thereof to improve its stability (particularly where the antibody or antigen-binding fragment thereof is an antibody fragment such as an Fv fragment).

In some embodiments, the antibodies or antigen-binding fragments thereof have amino acid alterations that alter the original glycosylation pattern of the antibody or antigen-binding fragment thereof. By “altering the original glycosylation pattern” is meant deleting one or more carbohydrate moieties found in the antibody or antigen-binding fragment thereof, and/or adding one or more glycosylation sites that are not present in the antibody or antigen-binding fragment thereof. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. Addition of glycosylation sites to the antibodies or antigen-binding fragments thereof that bind to HCMV is accomplished by altering the amino acid sequence such that it contains one or more of the above-described tripeptide sequences (for N-linked glycosylation sites). The alteration can also be made by the addition of, or substitution by, one or more serine or threonine residues to the sequence of the original antibody or antigen-binding fragment thereof (for O-linked glycosylation sites).

In some embodiments, the anti-HCMV antibodies or antigen-binding fragments thereof provided herein are deglycosylated or aglycosylated.

Where the antibody or antigen-binding fragment thereof comprises an Fc region, the carbohydrate(s) attached thereto can be altered. For example, antibodies with a mature carbohydrate structure that lacks fucose attached to an Fc region of the antibody or antigen-binding fragment thereof have been described. See, e.g., U.S. Patent Pubs. No. 2003/0157108; No. 2004/0093621. Antibodies with a bisecting N-acetylglucosamine (GlcNAc) in the carbohydrate attached to an Fc region of the antibody or antigen-binding fragment thereof are referenced in WO 03/011878; U.S. Pat. No. 6,602,684. Antibodies with at least one galactose residue in the oligosaccharide attached to an Fc region of the antibody or antigen-binding fragment thereof are reported in WO 97/30087. See also WO 98/58964 and WO 99/22764 concerning antibodies with altered carbohydrate attached to the Fc region thereof.

According to certain embodiments, the contemplated antibodies and antigen-binding fragments thereof also feature humanized frameworks for reduced immunogenicity. In certain embodiments, the CDRs of the contemplated antibody or antigen-binding fragment thereof are located in frameworks obtained from a human antibody or antigen-binding fragment thereof. In other embodiments, surface-exposed framework residues of the contemplated antibody or antigen-binding fragment thereof are replaced with framework residues of a human antibody or antigen-binding fragment thereof. The CDRs may also be located in murine or humanized frameworks linked to human constant regions (i.e., chimeric antibodies).

Techniques for humanization of murine antibodies are known to one of ordinary skill in the art and are generally reviewed in Safdari et al., (2013) Biotechnol. Genet. Eng. Rev., 29:175-86, hereby incorporated by reference in its entirety. Humanization of antibodies generally comprises grafting of CDRs (such as the CDRs disclosed herein) or conservative substituted variants thereof into an appropriate human variable region framework, for example, as disclosed in Jones et al. (1986) Nature 321, 522-525, hereby incorporated by reference in its entirety. Common methods used include, but are not limited to, framework-homology-based humanization, germline humanization, complementary determining regions (CDR)-homology-based humanization and specificity determining residues (SDR) grafting. Proper orientation of the CDRs in the humanized antibody is typically necessary and can be determined by, for example, evaluating the crystal structure of the humanized antibody.

In a preferred embodiment, the CDRs of a contemplated antibody or antigen-binding fragment thereof are located in frameworks that are a composite of two or more human antibodies. In such embodiments, the contemplated antibodies or antigen-binding fragments thereof comprise two or more sequence segments (“composites”) derived from V-regions of unrelated human antibodies that are selected to maintain monoclonal antibody sequences important for antigen-binding of the starting precursor anti-HCMV monoclonal antibody, and which have all been filtered for the presence of potential T cell epitopes using “in silico tools” (Holgate & Baker, IDrugs. 2009 April; 12 (4): 233-7). The close fit of human sequence segments with all sections of the starting antibody V regions and the elimination of CD4⁺ T cell epitopes prior to synthesis of the antibody or antigen-binding fragment thereof allow this technology to circumvent immunogenicity while maintaining optimal affinity and specificity through the prior analysis of sequences necessary for antigen-specificity (Holgate & Baker, 2009).

Antibodies with improved binding to the neonatal Fc receptor (FcRn), and increased half-lives, are described in WO 00/42072 and U.S. Patent Pub. No. 2005/0014934. These antibodies comprise an Fc region with one or more substitutions therein which improve binding of the Fc region to HCMV. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region comprising an antibody variant with improved HCMV binding comprises amino acid substitutions at one, two or three of positions 307, 380 and 434 of the Fc region thereof (Eu numbering of residues). In one embodiment, the antibody or antigen-binding fragment thereof has 307/434 mutations. Engineered antibodies that bind to HCMV with three or more (e.g., four) functional antigen-binding sites are also contemplated. See, e.g., U.S. Patent Pub. No. US 2002/0004587.

### Antibody Fragments and Types

In some embodiments of the aspects described herein, the anti-HCMV antibody fragment is a Fab fragment, which comprises or consists essentially of a variable (V_{L}) and constant (C_{L}) domain of the light chain and a variable domain (V_{H}) and the first constant domain (C_{H}1) of the heavy chain.

In some embodiments of the aspects described herein, the anti-HCMV antibody fragment is a Fab′ fragment, which refers to a Fab fragment having one or more cysteine residues at the C-terminus of the CHI domain.

In some embodiments of the aspects described herein, the anti-HCMV antibody fragment is an Fd fragment comprising or consisting essentially of V_{H} and C_{H}1 domains.

In some embodiments of the aspects described herein, the anti-HCMV antibody portion is an Fd′ fragment comprising V_{H} and C_{H}1 domains and one or more cysteine residues at the C-terminus of the C_{H}1 domain.

Single-chain Fv or scFv antibody fragments comprise or consist essentially of the V_{H} and V_{L} domains of antibody, such that these domains are present in a single polypeptide chain. Generally, an Fv polypeptide further comprises a polypeptide linker between the V_{H} and V_{L} domains, which allows the scFv to form the desired structure for antigen-binding. See, for example, Pluckthun, 113 Pharmacology Monoclonal Antibodies 269 (Rosenburg & Moore, eds., Springer-Verlag, New York, 1994). Accordingly, in some embodiments of the aspects described herein, the anti-HCMV antibody fragment is a Fv fragment comprising or consisting essentially of the V_{L} and Vu domains of a single arm of an antibody.

In some embodiments of the aspects described herein, the anti-HCMV antibody portion is a diabody comprising two antigen-binding sites, comprising a heavy chain variable domain (V_{H}) connected to a light chain variable domain (V_{L}) in the same polypeptide chain.

In some embodiments of the aspects described herein, the anti-HCMV antibody portion is a dAb fragment comprising or consisting essentially of a V_{H} domain.

In some embodiments of the aspects described herein, the anti-HCMV antibody portion is a F(ab′)₂ fragment, which comprises a bivalent fragment comprising two Fab′ fragments linked by a disulfide bridge at the hinge region.

Linear antibodies refer to the antibodies as described in Zapata et al., Protein Engin., 8 (10): 1057-1062 (1995). Briefly, these antibodies comprise a pair of tandem Fd segments (V_{H}—C_{H}1-V_{H}-C_{H}1), which, together with complementary light chain polypeptides, form a pair of antigen-binding regions. Linear antibodies can be bispecific or monospecific. In some embodiments of the aspects described herein, the anti-HCMV antibody fragment is a linear antibody comprising a pair of tandem Fd segments (V_{H}—C_{H}1-V_{H}-C_{H}1) which, together with complementary light chain polypeptides, form a pair of antigen-binding regions.

Various techniques have been developed and are available for the production of antibody fragments. Traditionally, these fragments were derived via proteolytic digestion of intact antibodies. See, e.g., Morimoto et al., 24 J. Biochem. Biophys. Meths. 107 (1992); Brennan et al., 229 Science 81 (1985). However, these fragments can now be produced directly by recombinant host cells. For example, antibody fragments can be isolated from the antibody phage libraries discussed herein. Alternatively, Fab′-SH fragments can be directly recovered from E. coli and chemically coupled to form F (ab′)₂ fragments (Carter et al., 1992). According to another approach, F (ab′)₂ fragments can be isolated directly from recombinant host cell culture. Other techniques for the production of antibody fragments will be apparent to the skilled practitioner. In other embodiments, the antibody fragment of choice is a single chain Fv fragment (scFv). See, for example, WO 93/16185.

Contemplated antibodies or antigen-binding fragments may have all types of constant regions, including IgA1, IgA2, IgM, IgG, IgD, and IgE, and any isotype, including IgG1, IgG2, IgG3, and IgG4. In one embodiment, the human isotype IgG1 is used. In one embodiment, the human isotype IgG2 is used. In another embodiment, the human isotype IgG4 is used. Light chain constant regions can be λ or κ. The antibody or antigen-binding fragment thereof may comprise sequences from more than one class or isotype.

Also disclosed herein are chimeric antigen receptor T-cells (CAR T-cells) that bind to HCMV. In one embodiment, one or more of the CDRs of an anti-HCMV antibody disclosed herein are grafted onto a chimeric antigen receptor (CAR) on a T-cell.

In some embodiments, the anti-HCMV antibody or antigen-binding fragment thereof is an isolated antibody or antigen-binding fragment thereof. The terms “purified” or “isolated” antibody, peptide, polypeptide, or protein refers to a peptide, polypeptide, or protein, as used herein, may refer to a peptide, polypeptide, or protein that has been separated from other proteins, lipids, and nucleic acids with which it is naturally associated. The polypeptide/protein can constitute at least 10% (i.e., any percentage between 10% and 100%, e.g., 20%, 30%, 40%, 50%, 60%, 70%, 80%, 85%, 90%, 95%, and 99%) by dry weight of the purified preparation. Purity can be measured by any appropriate standard method, for example, by column chromatography, polyacrylamide gel electrophoresis, or HPLC analysis. An isolated polypeptide/protein (e.g., anti-HCMV antibodies) described in herein can be produced by recombinant DNA techniques.

### Antibody Binding

As used herein, “binding” of an antibody or antigen-binding fragment thereof to HCMV or an epitope on the surface of HCMV includes the selective interaction of the antibody or antigen-binding fragment thereof with HCMV. Binding therefore includes, e.g., primary and secondary interactions including hydrogen bonds, ionic interactions, salt bridges, as well as hydrophilic and hydrophobic interactions.

As used herein, “affinity”, represented by the equilibrium constant for the dissociation (K_{D}) of an antigen with an antigen-binding protein, is a measure of the binding strength between an antigenic determinant and an antigen-binding site on the antigen-binding protein, such as an antibody or antibody fragment thereof. The smaller the value of the K_{D}, the stronger the binding strength between an antigenic determinant and the antigen-binding molecule. Alternatively, the affinity can also be expressed as the affinity constant (KA), which is 1/K_{D}). As will be clear to the skilled person, affinity can be determined in a manner known per se, depending on the specific antigen of interest.

In certain embodiments, the anti-HCMV antibodies or antigen-binding fragments thereof described herein bind to HCMV with a K_{D} of 10⁻⁵ to 10⁻¹² mol/l, 10⁻⁶ to 10⁻¹² mol/l, 10⁻⁷ to 10⁻¹² mol/l, 10⁻⁸ to 10⁻¹² mol/l, 10⁻⁹ to 10⁻¹² mol/l, 10⁻¹⁰ to 10⁻¹² mol/l, or 10⁻¹¹ to 10⁻¹² mol/l. In other embodiments, the anti-HCMV antibodies or antigen-binding fragments thereof described herein bind to HCMV with a K_{D} of 10⁻⁵ to 10⁻¹¹ mol/l, 10⁻⁶ to 10⁻¹¹ mol/l, 10⁻⁷ to 10⁻¹¹ mol/l, 10⁻⁸ to 10⁻¹¹ mol/l, 10⁻⁹ to 10⁻¹¹ mol/l, or 10⁻¹⁰ to 10⁻¹¹ mol/l. In other embodiments, the anti-HCMV antibodies or antigen-binding fragments thereof described herein bind to HCMV with a Kp of 10⁻⁵ to 10⁻¹⁰ mol/l, 10⁻⁶ to 10⁻¹⁰ mol/l, 10⁻⁷ to 10⁻¹⁰ mol/l, 10⁻⁸ to 10⁻¹⁰ mol/l, or 10⁻⁹ to 10⁻¹⁰ mol/l. In other embodiments, the anti-HCMV antibodies or antigen-binding fragments thereof described herein bind to HCMV with a K_{D} of 10⁻⁵ to 10⁻⁸ mol/l, 10⁻⁶ to 10⁻⁸ mol/l, or 10⁻⁷ to 10⁻⁸ mol/l.

Provided herein are antibodies and antigen-binding fragments thereof that bind specifically to HCMV.

The term “specificity” herein refers to the ability of an antibody or antigen-binding fragment thereof, such as an anti-HCMV antibody or antigen-binding fragment thereof, to recognize an HCMV epitope, while only having little or no detectable reactivity with other epitopes. Specificity can be relatively determined by competition assays or by epitope identification/characterization techniques described herein or their equivalents known in the art.

As used herein, an “epitope” can be formed both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a particular spatial conformation. An “epitope” includes the unit of structure conventionally bound by an immunoglobulin V_{H}/V_{L} pair. Epitopes define the minimum binding site for an antibody or antigen-binding fragment thereof, and thus represent the target of specificity of an antibody or antigen-binding fragment thereof. In the case of a single domain antibody, an epitope represents the unit of structure bound by a variable domain in isolation.

Provided therein are anti-HCMV antibodies or antigen-binding portions thereof provided herein that are capable of binding to a number of different structures on the surface of HCMV, including glycoprotein gH and glycoprotein gL.

Provided herein are anti-HCMV antibodies or antigen-binding portions thereof that specifically bind to glycoprotein gH. Provided herein are anti-HCMV antibodies or antigen-binding portions thereof that specifically bind to glycoprotein gL. Provided herein are anti-HCMV antibodies or antigen-binding portions thereof that specifically bind to glycoprotein gL and to glycoprotein gH.

In one embodiment, the contemplated antibody or antigen-binding fragment specifically binds to the same epitope as antibody 13G1, 4E1, 15G11, and/or 9A12.

In one embodiment, the contemplated antibody or antigen-binding fragment specifically competes with antibody 13G1, 4E1, 15G11, and/or 9A12 for binding to HCMV.

In some embodiments, an antibody disclosed herein blocks binding of HCMV gL/gH to a cellular surface protein. In some embodiments, an antibody disclosed herein interrupts a fusion-triggering signal to gB.

As used herein, a “blocking” antibody or an antibody “antagonist” is one that inhibits or reduces biological activity of the antigen to which it binds. Inhibition of activity and inhibition of binding includes partial inhibition. Methods for the identification of anti-HCMV antibodies o antigen-binding fragments thereof that block gH/gL interactions are described herein and are known to one skilled in the art. For instance, competing, cross-blocking, and cross-blocked antibodies can be identified using any suitable method known in the art, including competition ELISAs or BIACORE® assays where binding of the competing or cross-blocking antibody to human HCMV prevents the binding of an antibody disclosed herein or vice versa.

### Antibody Conjugates

In some embodiments of the aspects described herein, the antibody or antigen-binding fragment thereof that bind to HCMV are conjugated to a functional moiety. Examples of useful functional moieties include, but are not limited to, a blocking moiety, a detectable moiety, a diagnostic moiety, a targeting, and a therapeutic moiety.

Exemplary blocking moieties include moieties of sufficient steric bulk and/or charge such that reduced glycosylation occurs, for example, by blocking the ability of a glycosidase to glycosylate the antibody or antigen-binding fragment thereof. The blocking moiety may additionally or alternatively, reduce effector function, for example, by inhibiting the ability of the Fc region to bind a receptor or complement protein. Preferred blocking moieties include cysteine adducts and PEG moieties.

In a preferred embodiment, the blocking moiety is a cysteine, preferably a cysteine that has associated with a free cysteine, e.g., during or subsequent to the translation of the Fc containing polypeptide, e.g., in cell culture. Other blocking cysteine adducts include cystine, mixed disulfide adducts, or disulfide linkages.

In another preferred embodiment, the blocking moiety is a polyalkylene glycol moiety, for example, a PEG moiety and preferably a PEG-maleimide moiety. Preferred pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight, but these characteristics can affect the biological properties. Polymer average molecular weights particularly useful for decreasing clearance rates in pharmaceutical applications are in the range of 2,000 to 35,000 Daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically active molecule.

Examples of detectable moieties which are useful in the methods and antibodies and antigen-binding fragments thereof contemplated herein include fluorescent moieties or labels, imaging agents, radioisotopic moieties, radiopaque moieties, and the like, e.g., detectable labels such as biotin, fluorophores, chromophores, spin resonance probes, or radiolabels. Exemplary fluorophores include fluorescent dyes (e.g. fluorescein, rhodamine, and the like) and other luminescent molecules (e.g. luminal). A fluorophore may be environmentally-sensitive such that its fluorescence changes if it is located close to one or more residues in the modified protein that undergo structural changes upon binding a substrate (e.g., dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties which are useful in the methods and antibodies and antigen-binding fragments thereof contemplated herein include detectable moieties suitable for revealing the presence of a disease or disorder. Typically, a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties which are useful in the methods and antibodies and antigen-binding fragments thereof contemplated herein include, for example, anti-viral agents. The functional moiety may also have one or more of the above-mentioned functions.

Other types of functional moieties are known in the art and can be readily used in the methods and compositions of disclosed herein based on the teachings contained herein.

### Nucleic Acids

Also provided herein are nucleic acids encoding the anti-HCMV antibodies and antigen-binding fragments thereof disclosed herein, as well as vectors, host cells, and expression systems.

The term “nucleic acid” as used herein refers to a polymeric form of nucleotides of any length, either ribonucleotides or deoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The nucleic acids encoding anti-HCMV antibodies and antigen-binding fragments thereof may be, e.g., DNA, cDNA, RNA, synthetically produced DNA or RNA, or a recombinantly produced chimeric nucleic acid molecule comprising any of those polynucleotides either alone or in combination. For example, provided is an expression vector comprising a polynucleotide sequence encoding an anti-HCMV antibody or antigen-binding fragment thereof described herein operably linked to expression control sequences suitable for expression in a eukaryotic and/or prokaryotic host cell.

The term “vector” refers to a vehicle capable of transporting another nucleic acid to which it has been linked. A “vector” includes, but is not limited to, a viral vector, a plasmid, an RNA vector or a linear or circular DNA or RNA molecule which may consist of a chromosomal, non-chromosomal, semi-synthetic or synthetic nucleic acids. The vector can be a nucleic acid and or viral particle. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus.

A variety of expression vectors have been developed for the efficient synthesis of antibodies and antigen-binding fragments thereof in prokaryotic cells such as bacteria and in eukaryotic systems, including but not limited to yeast and mammalian cell culture systems have been developed. The vectors can comprise segments of chromosomal, non-chromosomal and synthetic DNA sequences.

Also provided are cells comprising expression vectors for the expression of the contemplated anti-HCMV antibodies or antigen-binding fragments thereof.

In one aspect, provided is a nucleic acid encoding an anti-HCMV antibody or antigen-binding fragment thereof disclosed herein. The sequences encoding the heavy chain variable region and the light chain variable region of an anti-HCMV antibody or antigen-binding fragment thereof disclosed herein may be located on the same nucleic acid molecules or on different nucleic acid molecules.

In one embodiment, provided is a nucleic acid encoding the heavy chain variable region of an HCMV antibody or antigen-binding fragment thereof disclosed herein. In one embodiment, provided is a nucleic acid encoding the light chain variable region of an anti-HCMV antibody or antigen-binding fragment thereof disclosed herein.

In one embodiment, provided is a vector or set of vectors comprising a sequence encoding the heavy chain variable region of an anti-HCMV antibody or antigen-binding fragment thereof disclosed herein and the light chain variable region of an anti-HCMV antibody or antigen-binding fragment thereof disclosed herein. In one embodiment, the heavy chain variable region is encoded by a first vector and the light chain variable region is encoded by a second vector.

Provided herein are nucleic acids encoding the antibodies in Table 2.

TABLE 2
Nucleic acid sequences of antibodies 13G1, 14E1, 15G11, and 9A12

| | 13G1 | 14E1 | 15G11 | 9A12 |
|---|---|---|---|---|
| Description | (SEQ ID NO) | (SEQ ID NO) | (SEQ ID NO) | (SEQ ID NO) |
| CDR1H | 1 | 9 | 17 | 25 |
| CDR2H | 2 | 10 | 18 | 26 |
| CDR3H | 3 | 11 | 19 | 27 |
| CDR1L | 4 | 12 | 20 | 28 |
| CDR2L | gctgcatcc | gctgcatcc | ggtgcatcc | gctgcatcc |
| CDR3L | 6 | 14 | 22 | 30 |
| VH | 7 | 15 | 23 | 31 |
| VL | 8 | 16 | 24 | 32 |

Provided herein is a vector or set of vectors encoding an antibody or antigen-binding fragment thereof which binds to HCMV, the antibody or antigen-binding fragment comprising a heavy chain variable region and a light chain variable region, wherein:
(a) the sequence encoding the heavy chain variable region comprises a sequence that is at least 80%, at least 85%, at least 90%, at least 95%, at least 96%, at least 97%, at least 98%, or at least 99% identical to SEQ ID NO:7 and the sequence encoding the light chain variable region comprises a sequence that is at least 80%, at least 85%, at least 90%, at least 95%, at least 96%, at least 97%, at least 98%, or at least 99% identical to SEQ ID NO:8;
(b) the sequence encoding the heavy chain variable region comprises a sequence that is at least 80%, at least 85%, at least 90%, at least 95%, at least 96%, at least 97%, at least 98%, or at least 99% identical to SEQ ID NO:15 and the sequence encoding the light chain variable region comprises a sequence that is at least 80%, at least 85%, at least 90%, at least 95%, at least 96%, at least 97%, at least 98%, or at least 99% identical to SEQ ID NO:16;
(c) the sequence encoding the heavy chain variable region comprises a sequence that is at least 80%, at least 85%, at least 90%, at least 95%, at least 96%, at least 97%, at least 98%, or at least 99% identical to SEQ ID NO:23 and the sequence encoding the light chain variable region comprises a sequence that is at least 80%, at least 85%, at least 90%, at least 95%, at least 96%, at least 97%, at least 98%, or at least 99% identical to SEQ ID NO:24; or
(d) the sequence encoding the heavy chain variable region comprises a sequence that is at least 80%, at least 85%, at least 90%, at least 95%, at least 96%, at least 97%, at least 98%, or at least 99% identical to SEQ ID NO:31 and the sequence encoding the light chain variable region comprises a sequence that is at least 80%, at least 85%, at least 90%, at least 95%, at least 96%, at least 97%, at least 98%, or at least 99% identical to SEQ ID NO:32.

Provided herein is a vector or set of vectors encoding an antibody or antigen-binding fragment thereof which binds to HCMV, the antibody or antigen-binding fragment comprising a heavy chain variable region and a light chain variable region, wherein:
(a) the sequence encoding the heavy chain variable region comprises SEQ ID NO:7 and the sequence encoding the light chain variable region comprises SEQ ID NO:8;
(b) the sequence encoding the heavy chain variable region comprises SEQ ID NO:15 and the sequence encoding the light chain variable region comprises SEQ ID NO:16;
(c) the sequence encoding the heavy chain variable region comprises SEQ ID NO:23 and the sequence encoding the light chain variable region comprises SEQ ID NO:24; or
(d) the sequence encoding the heavy chain variable region comprises SEQ ID NO:31 and the sequence encoding the light chain variable region comprises SEQ ID NO:32.

The nucleic acid sequences may have conservative substitutions and/or may be codon optimized.

As used herein, codon optimization refers to an in vitro mutagenesis of a nucleic acid to increase or maximize expression of a gene (e.g. a transgene relative to the unmodified nucleic acid, without changing (or with minimal change) to the amino acid sequence of the synthesized protein, i.e. synonymous mutations. Codon optimization can affect protein expression rates up to 1,000× fold, particularly by favoring efficient soluble protein expression. The codons changed are typically ones not generally used by the host cell translation system. Codon bias/codon usage frequency depends on the host organism, and is described, for example, in U.S. Pat. No. 8,326,547, hereby incorporated by reference in its entirety.

### Antibody Preparation and Expression Systems

The antibodies or antigen-binding fragments thereof disclosed herein are typically produced by recombinant expression. Nucleic acids encoding light and heavy chain variable regions, optionally linked to constant regions, are inserted into expression vectors. The light and heavy chains can be cloned in the same or different expression vectors. The DNA segments encoding immunoglobulin chains are operably linked to control sequences in the expression vector(s) that ensure the expression of immunoglobulin polypeptides. Expression control sequences include, but are not limited to, promoters (e.g., naturally associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

The expression of the antibodies and antigen-binding fragments contemplated herein can occur in either prokaryotic or eukaryotic cells. Suitable hosts include bacterial or eukaryotic hosts, including yeast, insects, fungi, bird, and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used.

E. coli is one prokaryotic host particularly useful for cloning the polynucleotides (e.g., DNA sequences). Other microbial hosts suitable for use include bacilli, such as Bacillus subtilus, and other enterobacteriaceae, such as Salmonella, Serratia, and various Pseudomonas species.

Other microbes, such as yeast, are also useful for expression. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an anti-HCMV antibody or antigen-binding fragment thereof disclosed herein with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989).

Any of a series of yeast gene expression systems incorporating promoter and termination elements from the actively expressed genes coding for glycolytic enzymes produced in large quantities when yeast are grown in mediums rich in glucose can be utilized to obtain recombinant anti-HCMV antibodies or antigen-binding fragments disclosed herein. Known glycolytic genes can also provide very efficient transcriptional control signals. For example, the promoter and terminator signals of the phosphoglycerate kinase gene can be utilized.

Production of anti-HCMV antibodies or antigen-binding fragments thereof in insects can be achieved. For example, by infecting the insect host with a baculovirus engineered to express a transmembrane polypeptide by methods known to those of skill. See Ausubel et al., 1987, 1993.

In addition to microorganisms, mammalian tissue culture may also be used to express and produce the antibodies or antigen-binding fragments thereof disclosed herein (e.g., polynucleotides encoding immunoglobulins or fragments thereof). See Winnacker, From Genes to Clones, VCH Publishers, N.Y., N.Y. (1987). Eukaryotic cells are actually preferred, because a number of suitable host cell lines capable of secreting heterologous proteins (e.g., intact immunoglobulins) have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, nucleotide sequences encoding antibodies or antigen-binding fragments thereof can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for light and/or heavy chains in operable linkage with a promoter and enhancer from a mammary gland specific gene, such as casein or beta lactoglobulin.

Additionally, plants have emerged as a convenient, safe and economical alternative main-stream expression systems for recombinant antibody production, which are based on large scale culture of microbes or animal cells. Antibodies or antigen-binding fragments thereof can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560 and 6,512,162; and WO 0129242. Several plant-derived antibodies have reached advanced stages of development, including clinical trials (see, e.g., Biolex, NC).

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

The antibodies and antigen-binding fragments thereof disclosed herein can be expressed using a single vector or two vectors. When the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms disclosed herein can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

Provided herein is a method of making an antibody or antigen-binding fragment thereof which binds to HCMV, the method comprising: (i) providing a cell comprising one or more nucleic acid molecules encoding an anti-HCMV antibody or antigen-binding fragment disclosed herein; (ii) expressing in the cell at least one of a heavy variable chain, a light variable chain, or combinations thereof; and (iii) collecting the antibody or antigen-binding fragment thereof. In some embodiments, the antibody or antigen-binding fragment thereof is further purified.

### Kits and Methods of Use

Provided herein are kits for detecting HCMV present in a sample. These kits may comprise an anti-HCMV antibody or antigen-binding portion thereof disclosed herein and various reagents, for example, reagents that aid in detection of binding between the anti-HCMV antibody and an epitope present on HCMV or an antigenic fragment thereof.

The term “biological sample” as used herein may refer to a sample obtained from an organism (e.g., patient) or from components (e.g., cells) of an organism. The sample may be of any biological tissue, cell(s) or fluid. The sample may be a “clinical sample” which is a sample derived from a subject, such as a human patient. Such samples include, but are not limited to, saliva, sputum, blood, blood cells (e.g., white cells), bodily fluids, lavages, pancreatic juices, gastric juices, discharges, CSF, lymph amniotic fluid, plasma, semen, bone marrow, and tissue or fine needle biopsy samples, urine, stool, peritoneal fluid, and pleural fluid, or cells therefrom, and any combinations thereof. Biological samples may also include sections of tissues such as frozen sections taken for histological purposes. A biological sample may also be referred to as a “patient sample.” A biological sample may also include a substantially purified or isolated protein, membrane preparation, or cell culture.

The kits may be used in vitro assays, such as immunoassays, e.g. enzyme immune assays (EIA), enzyme linked immunosorbent assay (ELISA), ELISPOT (enzyme-linked immunospot), radioimmunoassays (RIAs), immunofluorescence, and other assays known in the art, including but not limited to Western Blot analysis and/or immunoprecipitation methods. The in vitro assays may be competitive, or indirect, such as in a sandwich assay, or may be an antibody capture method. For example, in a direct ELISA, a buffered solution of an antigen, e.g., a sample containing HCMV or an antigenic fragment thereof (e.g., a biological sample containing or suspected of containing HCMV) is added to a well of a microtiter plate, e.g. a 96-well plate. A solution of non-reacting protein, e.g. bovine serum albumin or casein is then added to the well. The anti-HCMV antibody or antigen-binding portions thereof conjugated to a reporter molecule enzyme is added, e.g. conjugated to horse-radish peroxidase, although that is not necessarily the enzyme, as other common enzymes include alkaline phosphatase, or β-D-galactosidase, although other enzymes are conceivable and considered embodied by the present disclosure. A substrate for the enzyme is then added, which leads to a detectable signal. For example, adding TMB to horseradish peroxidase leads to a colored product, in which case the ELISA is a colorimetric assay. ELISAs may be run in a qualitative or quantitative format. Qualitative results provide a simple positive or negative result (yes or no) for a sample. The cutoff between positive and negative is determined by the analyst and may be statistical. Sandwich ELISAs generally follow the following protocol. Capture anti-HCMV antibody or antigen-binding portions thereof is bound to (i.e. “immobilized”) on a substrate, e.g. a mictotiter plate. Antigen-containing sample (i.e. sample containing HCMV or an antigenic fragment thereof, is then added to the substrate at which point it is captured by the anti-HCMV antibodies. The substrate is then washed to remove unbound antigen. A second anti-HCMV antibody or antigen-binding portions thereof is added, which binds to a different epitope on HCMV. The second anti-HCMV antibody or antigen-binding portions thereof is bound to a reporter molecule, e.g., an enzyme, although the reporter molecule may be any molecule which leads to a detectable signal. The plate may be washed a second time, and in those instances where the reporter molecule is an enzyme, a substrate may be added, e.g., TMB, that results in a detectable signal (also a colorimetric assay). A third type of common ELISA is competitive ELISA. In these embodiments, unlabeled anti-HCMV antibody or antigen-binding portions thereof is incubated in the presence of an antigen-containing sample (i.e. sample containing HCMV or an antigenic fragment thereof), which are then added to an antigen-coated well. The plate is washed to remove unbound antibodies. A secondary antibody is added that is specific to the primary antibody, e.g., a secondary antibody specific to anti-HCMV antibodies. The secondary antibody is bound to a reporter molecule, as described herein, such as an enzyme (or any other molecule that may lead to a detectable signal). Some competitive ELISA utilize labeled antigens rather than labeled antibodies; the less antigen in the sample, the more labeled antigen is retained and the stronger a detectable signal results.

Other forms of common in vitro assays include radioimmunoassays (RIAs). Typically, a known quantity of an antigen is linked to a radioactive tracer, e.g. I-125 although others are suitable for use, which is then mixed with a known amount of antibody specific for the antigen, e.g., anti-HCMV antibodies or antigen-binding portions thereof. Then, a sample containing unknown quantity of an antigen is added, (e.g., a biological sample that contains or is suspected of containing HCMV or an antigenic fragment thereof) is added. This is a direct competitive for specific binding; as the concentration of unlabeled antigen is increased, the binding between the anti-HCMV antibodies and the labeled standard is decreased, which is directly measurable by measuring radioactivity. Other assays are known and a person of ordinary skill in the art would readily recognize their applicability.

In some embodiments, provide is a method of detecting HCMV or an antigenic fragment thereof in a sample. Such methods may utilize any of the assays described herein, or others that are known in the art. Several of the assays described herein are capable of quantifying the amount of antigen present in a sample, and so accordingly, in some embodiments, the present disclosure is directed to methods of quantifying the amount of HCMV or antigenic fragments thereof present in a sample, e.g., a biological sample.

The assays containing anti-HCMV antibodies or antigen-binding portions thereof of the present disclosure may or may not be utilized for diagnostic purposes. Accordingly, in some embodiments, provided are methods of diagnostic use of the anti-HCMV antibodies or antigen-binding portions thereof of the present disclosure. Because of the specificity of the anti-HCMV antibodies or antigen-binding portions thereof of the present disclosure, immunoassays containing anti-HCMV antibodies or antigen-binding portions thereof of the present disclosure may be sufficient to diagnose an individual as having an active or latent infection of HCMV. The antibodies or antigen-binding portions thereof need not be restricted to any particular epitopes, so long as the antibodies or antigen-binding portions thereof used are specific to HCMV. For example, in a sandwich assay, the first anti-HCMV antibody or antigen-binding portions thereof may bind to a first epitope, such as a gH or gL glycoprotein, and the second anti-HCMV antibody or antigen-binding portions thereof (which is bound to a reporter molecule) may bind to a second epitope; for example, but not necessarily, if the first anti-HCMV antibody or antigen-binding portions thereof specifically binds to the glycoprotein gH, the second may bind to glycoprotein gL, and the reverse is true as well. In some embodiments, the first antibody or antigen-binding portions thereof and second antibody or antigen-binding portions thereof may bind to the same antigen/glycoprotein, e.g., both may bind to glycoprotein gH and/or gL, or in some embodiments may bind to completely different antigens on surface of HCMV viral envelope.

Because of the distance between binding sites, these antibodies or antigen-binding portions thereof may be suitable for use in those immunoassays, e.g., sandwich assays, in which multiple binding sites on the same target (e.g., HCMV or an antigenic fragment thereof) are necessary. Or, in some embodiments, one may target a totally different antigen, e.g., the first anti-HCMV antibody or antigen-binding portions thereof targets an epitope on glycoprotein gH and/or gL and the second anti-HCMV antibody targets an epitope elsewhere, for example but not necessarily glycoprotein gB, gO, UL128, UL130, and/or UL131a and the antigenic fragments thereof of the corresponding antigens.

Methods for Modulating gH/gL Activity

In one aspect, provided are methods of using the antibodies and antigen-binding fragments thereof described herein for decreasing the interaction between gH/gL and cellular surface proteins. Such cellular surface proteins may include, but are not limited to, Nectin 1, EphA2, Nrp2, PDGFRalpha.

### Methods of Treatment

In one aspect, provided are anti-HCMV antibodies and antigen-binding fragments thereof that are also useful for the treatment of subjects in need thereof or for the prevention of disease.

By “subject” is meant a mammal, including, but not limited to, a human or non-human mammal, such as a bovine, equine, canine, ovine, or feline, etc. Individuals and patients are also subjects herein.

The terms “treat,” “treated,” “treating,” or “treatment” as used herein refer to therapeutic treatment, wherein the object is to slow down (lessen) an undesired physiological condition, disorder or disease, or to obtain beneficial or desired clinical results. Beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total) or enhancement or improvement of the condition, disorder or disease. Treatment includes eliciting a clinically significant response without excessive levels of side effects. Treatment also includes prolonging survival as compared to expected survival if not receiving treatment.

The terms “prevent”, “prevention”, and the like refer to acting prior to overt disease or disorder onset, to prevent the disease or disorder from developing or to minimize the extent of the disease or disorder, or slow its course of development.

Provided is a method of treating an HCMV infection in a subject in need thereof, the method comprising administering to the subject an antibody or antigen-binding fragment thereof disclosed herein. In some embodiments, the method further comprises administering to the individual at least one additional anti-HCMV antibody or antigen-binding portion thereof. In some embodiments the at least one additional anti-HCMV antibody or antigen-binding portion thereof is an antibody or antigen-binding fragment thereof of disclosed herein.

Provided is an anti-HCMV antibody or antigen-binding portion thereof disclosed herein for use in treating an HCMV infection in a subject in need thereof. Provided is a set of anti-HCMV antibodies or antigen-binding portions thereof disclosed herein for use in treating an HCMV infection in a subject in need thereof.

Provided is an anti-HCMV antibody or antigen-binding portion thereof disclosed herein for the manufacture of a medicament for treating an HCMV infection in a subject in need thereof. Provided is a set of anti-HCMV antibodies or antigen-binding portions thereof disclosed herein for the manufacture of a medicament for treating an HCMV infection in a subject in need thereof.

Provided is an anti-HCMV antibody or antigen-binding portion thereof disclosed herein for use as a medicament. Provided is a set of anti-HCMV antibodies or antigen-binding portions thereof disclosed herein for use as a medicament.

Provided is a method of treating an HCMV infection in a subject in need thereof, the method comprising administering to the subject a pharmaceutical composition comprising an antibody or antigen-binding fragment thereof disclosed herein. In some embodiments, the method further comprises administering to the individual at least one additional pharmaceutical composition comprising an anti-HCMV antibody or antigen-binding portion thereof. In some embodiments the at least one additional anti-HCMV antibody or antigen-binding portion thereof is an antibody or antigen-binding fragment thereof of disclosed herein.

Provided is a pharmaceutical composition comprising an anti-HCMV antibody or antigen-binding portion thereof disclosed herein for use in treating an HCMV infection in a subject in need thereof. Provided is a pharmaceutical composition comprising a set of anti-HCMV antibodies or antigen-binding portions thereof disclosed herein for use in treating an HCMV infection in a subject in need thereof.

Provided is a pharmaceutical composition comprising an anti-HCMV antibody or antigen-binding portion thereof disclosed herein for the manufacture of a medicament for treating an HCMV infection in a subject in need thereof. Provided is a pharmaceutical composition comprising a set of anti-HCMV antibodies or antigen-binding portions thereof disclosed herein for the manufacture of a medicament for treating an HCMV infection in a subject in need thereof.

Provided is a pharmaceutical composition comprising an anti-HCMV antibody or antigen-binding portion thereof disclosed herein for use as a medicament. Provided is a pharmaceutical composition comprising a set of anti-HCMV antibodies or antigen-binding portions thereof disclosed herein for use as a medicament.

Provided herein are methods in which a therapeutically effective amount of an antibody or antigen-binding portions thereof set forth herein is administered to a mammal in need thereof. Although antibodies or antigen-binding portions thereof set forth herein are particularly useful for administration to humans, they may be administered to other mammals as well. The term “mammal” as used herein is intended to include, but is not limited to, humans, laboratory animals, domestic pets and farm animals. “Therapeutically effective amount” means an amount of antibody or antigen-binding portions thereof set forth herein that, when administered to a mammal, is effective in producing the desired therapeutic effect.

In some embodiments, the anti-HCMV antibodies or antigen-binding portions thereof of the present disclosure may be co-administered with one or more additional treatments for HCMV, e.g., co-administered with one or more antivirals and/or additional anti-HCMV antibodies or antigen-binding portions thereof, including but not limited to additional anti-HCMV antibodies or antigen-binding portions thereof disclosed herein. The most common antiviral treatment for HCMV is ganciclovir, and accordingly in one embodiment the anti-HCMV antibodies or antigen-binding portions thereof of the present disclosure may be co-administered with ganciclovir. Other antivirals that would be acceptable include valganciclovir, forscarnet, and cidofovir, either in combination or alone, including in combination with ganciclovir. Additionally, co-administration may or may not be with additional anti-HCMV antibodies or antigen-binding portions thereof. The anti-HCMV antibodies or antigen-binding portions thereof of the present disclosure may be administered with a variety of additional existing antibodies, such as CytoGam®.

In some embodiments, provided is a method of treating an HCMV infection in a subject in need thereof comprising, the method comprising administering to the subject (i) an antibody or antigen-binding fragment thereof disclosed herein and (ii) at least one additional antiviral composition. In some embodiments, the at least one additional antiviral composition is selected from the group consisting of ganciclovir, valganciclovir, foscarnet, cidofovir, and combinations thereof.

The antibody or antigen-binding fragment thereof disclosed herein and the second antiviral composition (which could, for example, be a second anti-HCMV antibody or antigen-binding fragment thereof), can be administered consecutively or concurrently. The antibody or antigen-binding fragment thereof disclosed herein, and the second antiviral composition do not need to be present in the same packaging or composition.

In one aspect, provided is a method of preventing an HCMV infection in a subject comprising administering to the subject the antibody or antigen-binding fragment thereof disclosed herein.

Provided is an anti-HCMV antibody or antigen-binding portion thereof disclosed herein for use in preventing an HCMV infection in a subject in need thereof. Provided is a set of anti-HCMV antibodies or antigen-binding portions thereof disclosed herein for use in preventing an HCMV infection in a subject in need thereof.

Provided is an anti-HCMV antibody or antigen-binding portion thereof disclosed herein for the manufacture of a medicament for preventing an HCMV infection in a subject in need thereof. Provided is a set of anti-HCMV antibodies or antigen-binding portions thereof disclosed herein for the manufacture of a medicament for preventing an HCMV infection in a subject in need thereof.

Provided herein is a method of diagnosing a subject as having an HCMV infection comprising:
(i) identifying a subject;
(ii) obtaining from the subject a biological sample containing HCMV or an antigenic fragment thereof;
(iii) contacting the sample with the antibody or antigen-binding fragment thereof disclosed herein;
(iv) detecting the presence of specific binding of the antibody or antigen-binding fragment thereof to HCMV, or an antigenic fragment thereof; and
(v) diagnosing the subject as having an HCMV infection.

### Pharmaceutical Compositions

In another aspect, provided herein are pharmaceutically acceptable compositions that comprise a therapeutically effective amount of an anti-HCMV antibody or antigen-binding fragment thereof is described herein formulated together with one or more pharmaceutically acceptable excipients.

The active agent and excipient(s) may be formulated into compositions and dosage forms according to methods known in the art. The pharmaceutical compositions disclosed herein may be specially formulated in solid or liquid form, including those adapted for parenteral administration, for example, by subcutaneous, intratumoral, intramuscular or intravenous injection as, for example, a sterile solution or suspension.

Therapeutic compositions comprising antibodies or antigen-binding fragments thereof that bind to HCMV may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent is a compound which adds mass to a pharmaceutical formulation and contributes to the physical structure of the formulation in lyophilized form. Suitable bulking agents include mannitol, glycine, polyethylene glycol and sorbitol.

The use of a surfactant can reduce aggregation of the reconstituted protein and/or reduce the formation of particulates in the reconstituted formulation. The amount of surfactant added is such that it reduces aggregation of the reconstituted protein and minimizes the formation of particulates after reconstitution. Suitable surfactants include polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

Preservatives may be used in formulations disclosed herein. Suitable preservatives include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

The compositions comprising an antibody or antigen-binding fragment thereof and a pharmaceutically acceptable carrier may comprise the anti-HCMV antibodies or antigen-binding portions thereof set forth herein at various concentrations. For example, the compositions may comprise an antibody or antigen-binding fragment thereof at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions also may comprise an antibody or antigen-binding fragment thereof at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml.

In some embodiments, the compositions comprising the antibody or antigen-binding fragment thereof and the pharmaceutically acceptable carrier are lyophilized and provided in a composition for reconstitution prior to administration.

### Methods of Administration

Therapeutic compositions comprising the contemplated antibody or antigen-binding fragment thereof may be administered in any convenient manner, including by injection, transfusion, implantation or transplantation. The compositions described herein may be administered to a patient subcutaneously, intradermally, intratumorally, intranodally, intramedullary, intramuscularly, intracranially, by intravenous or intralymphatic injection, or intraperitoneally. In one embodiment, the cell compositions disclosed herein are preferably administered by intravenous injection.

In some embodiments, the amount of antibody administered is in the range of about 0.001 mg/kg to about 1000 mg/kg of patient body weight, and any range in between. Depending on the type and severity of the infection, about 0.1 mg/kg to about 50 mg/kg body weight (for example, about 0.1-15 mg/kg/dose) of antibody is an initial candidate dosage for administration to the patient, whether, for example, by one or more separate administrations, or by continuous infusion. The anti-HCMV antibodies or antigen-binding fragments thereof can be delivered relatively low volume rates, for example but not necessarily from about 0.001 ml/day to 10 ml/day so as to minimize tissue disturbance or trauma near the site where the formulation is released. The formulation may be released at a rate of, depending on the specific biological agent(s), at a low dose, e.g., from about 0.01 μg/hr or 0.1 μg/hr, 0.25 μg/hr, 1 μg/hr, generally up to about 200 μg/hr, or the formulation is delivered at a low volume rate e.g., a volume rate of from about 0.001 ml/day to about 1 ml/day, for example, 0.01 micrograms per day up to about 20 milligrams per day. Dosage depends on a number of factors such as potency, bioavailability, and toxicity of the active ingredient used (e.g. the anti-HCMV antibodies or antigen-binding portions thereof) and the requirements of the subject. The progress of this therapy is readily monitored by conventional methods and assays and based on criteria known to the physician or other persons of skill in the art. The above parameters for assessing successful treatment and improvement in the disease are readily measurable by routine procedures familiar to a physician.

In certain embodiments, the antibody or antigen-binding fragment thereof is administered to the mammal by intravenous infusion, i.e., introduction of the antibody or antigen-binding fragment thereof into the vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, a dose of a compound or a composition is administered to a subject every day, every other day, every couple of days, every third day, once a week, twice a week, three times a week, once every two weeks, or once a month. In other embodiments, two, three or four doses of a compound or a composition is administered to a subject every day, every couple of days, every third day, once a week, once every two weeks or once a month. In some embodiments, a dose(s) of a compound or a composition is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more.

It is to be understood that this disclosure is not limited to the particular molecules, compositions, methodologies, or protocols described, as these may vary. Any methods and materials similar or equivalent to those described herein can be used in the practice or testing of embodiments disclosed herein. It is further to be understood that the disclosure includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply.

To facilitate a better understanding of the present invention, the following examples of specific embodiments are given. The following examples should not be read to limit or define the entire scope of the invention.

### EXAMPLES

### Example 1: Materials and Methods for Examples 2-10

### Cell Lines, Antibodies, Plasmids, and Viruses

The MRC5 lung fibroblasts (ATCC #CCL-171), NHDF dermal fibroblasts, BHK (ATCC #CCL-10) and the U373 astrocytoma cell lines were cultured in Dulbecco's modified Eagle's medium (DMEM, Corning #10-013-CV). The ARPE-19 human retinal epithelial cells (ATCC #CRL-2302) were cultured in DMEM and F-12 medium (Gibco, #11765-054) mixed at 1:1 ratio. The trophoblast cell line, HTR-8/SVneo (ATCC, #CRL-3271) were obtained and cultured in RPMI medium (Corning, 10-041-CV). The DMEM, RPMI and DMEM/F-12 mediums were all supplemented with 10% fetal bovine serum (FBS), 1 mM HEPES (Corning, #25-060-CI), 100 U/mL of penicillin and 100 μg/mL of streptomycin (100× Pen/Strep, Corning, #30-002-CI). The embryonic kidney cell line Expi293F (Thermo Fisher, #A14527) were maintained using the serum free Expi293™ Expression Medium (Thermo Fisher, #A1435101), and kept in suspension by shaking at 125 rpm. The U373 cell lines which constitutively express the CMV glycoproteins gB, gH/gL, gHgLgO, and gHgLUL128 were generated as previously described (Gardner, T J. 2016 Nat Comm) and propagated in complete DMEM media. All cell lines were kept at 37° C. with 5% CO₂.

M2E10 (anti-IAV) and PY102 (anti-IAV) served as control antibodies. CytoGam®) was purchased from CSL Behring LLC (#NDC-44206-532-90). Monoclonal antibody W6/32 (anti-MHC-I) was purified from the supernatant of a hybridoma cell culture supernatant. MSL-109 was cloned based on the published nucleotide sequence, expressed in HEK-293 cells and enriched from the conditioned supernatant using a classical Ni-NTA purification system The polyclonal anti-gL immunoglobulins were generated in rabbits following inoculation with a peptide derived from the CMV TB40/E gL sequence (aa. 265-278, PAHSRYGPQAVDAR, SEQ ID NO:61). The following antibodies were purchased commercially; mouse anti-glyceraldehyde-3-phosphate dehydrogenase (GAPDH) (EMD Millipore, #MAB374), donkey anti-rabbit IgG-HRP (Invitrogen, #A16035) and donkey anti-mouse-HRP (Invitrogen, #A16017). The secondary antibodies used for detection in flow cytometry experiments were goat anti-mouse Alexa Fluor 647 (Invitrogen, #A21236), chicken-anti-rabbit Alexa Fluor 647 (Invitrogen, A21443) or chicken anti-human Alexa Fluor 647. The polyclonal anti-IE1 antibody was generated following immunization of rabbits with the linearized peptide sequence N′-KRKMDPDNPDEGPS-C′, SEQ ID NO:62.

The gH/gL plasmid used for vaccination was generated by cloning the gH and gL sequence of TB40E WT into a mammalian expression vector.

CMV virus was propagated in MRC5 or NHDF fibroblast cell lines and the virus was purified from infected cell supernatant and cell lysate following sonification to lyse the cells, then further purified by ultracentrifugation (20,000 rpm, 1.5 hrs, 25° C.) over a 20% sorbital (Fisher Scientific, #S459-500) cushion using a SW28 rotor (Bechman Coulter). Resulting virus was resuspended in PBS/BSA and stored at −80° C. The viruses were titered in ARPE-19 and either NHDF or MRC5 fibroblasts to determine the infectious units per microliter (IU/mL).

### Mouse Immunization, Hybridoma Fusion and Study Approval

12-week-old female mice (5/group) were divided into three immunization groups and received either 100 μg purified CMV (Merlin) formulated with 100 μg of Poly IC, 50 μg TB40E and 50 μg VHLE formulated with PolyIC, or electroporated with 100 μg of DNA plasmid encoding the gH/gL TB40E strain sequence. Following the prime (100 μg), mice immunized with purified virus were boosted 14 days post prime and 21 days post DNA immunization. Each group received a total of four boosts and blood was collected from the submandibular vein at 50 and 150 days post prime. Following sera neutralization analysis, three mice were selected from the DNA immunization group for hybridoma fusion and received two final boosts consisting of 50 μg of TB40E WT virus at −5 and −2 days before being euthanized by IACUC approved methods-CO₂ asphyxiation. The spleens were processed to single cell suspension and hybridomas were generated using the standard protocol. Briefly, the individual B cell clones were grown on a soft agar and selected for screening using a robotic ClonaCell Easy Pick instrument (Hamilton/Stem Cell Technology). Individual clones were expanded, and the supernatant was used to screen for binding to gH/gL. All animal studies were approved by the Icahn School of Medicine Institutional Animal Care and Use Committee (IACUC).

### Antibody Sequencing

For the sequencing of the variable heavy and kappa chains, RNA was extracted from the each hybridoma using Qiagen RNeasy Mini Kit (Qiagen, Valencia, CA), followed by first stand cDNA synthesis using random hexamers and poly-T primers (Integrated DNA Technologies, Caralville, IA) and SuperScript 3 reverse transcriptase (ThermoFisher Scientific, NY, NY). PCR with FastStart Taq DNA polymerase (Roche) was performed according to factory supplier using degenerate broad primers specific for 5′ UTR ends of heavy and kappa mouse V genes and specific IgG constant and kappa constant 3′ primers. Purified PCR product was then submitted for Sanger sequencing in both directions using same primers as PCR (GeneWiz, South Plainfield, NJ). Sequence results were blasted against the IMGT mouse databank of germline genes using V-Quest (http://imat.org) and specific 5′ and 3′ primers were built to the exact 5′ start of the V gene and the exact 3′ end of the J gene.

High-Throughput Expi293 gH/gL Binding Assay

Expi293F cells were transiently transfected to express gH/gL glycoproteins using Lipofectamine 3000 (L3000001, Thermo Fisher) and then incubated with supernatant from the hybridoma cell lines from each fusion. Binding was detected using an anti-mouse Fc Alexa Fluor 647 detection antibody and samples were run on a high-throughput flow cytometer (HTFC, Sartorius Group). Cells with a high mean fluorescence intensity were identified using FlowJo software (Tree Star, Inc.) and graphed using GraphPad Prism to create a heat map based on MFI.

### Isotyping

VelocImmune® animals produce chimeric antibodies with human variable genes and mouse constant genes. Isotyping for the constant gene of the antibodies was performed with the Mouse Immunoglobulin Isotyping Kit (BD, 550026) for flow cytometry, following the manufacturer's instructions.

### High-Throughput Neutralization Screening of Hybridoma Clones

AD169RBAD^{rUL131} or TB40E was pre-incubated with hybridoma supernatant (10 μl of supernatant and 40 μL of media containing virus; MOI 0.2) and incubated at 4° C. for 1 hr before being added to ARPE-19 cells (10,000 cells per well) for 2 hrs at 37° C., 5% CO₂. Following infection, the inoculum was removed and replaced with 100 μL complete DMEM/F-12 media. After an overnight incubation, cells were stained using the immunostaining protocol outlined below.

### Monoclonal Antibody Purification

Monoclonals were purified by FPLC on an AKTA pure FPLC systems using protein G affinity columns (HiTrap-1 ml, GE/Cytiva, #17-0404-01). The antibodies were dialyzed against PBS and quantitated by using a bicinchoninic acid (BCA) assay and by measuring the OD at 280 nm.

### Virus Neutralization

AD169R^{BADrUL131}, TB40E, TR, or Towne was pre-incubated with monoclonal antibodies (starting dilution of 100 μg/mL antibody) was diluted using 5 fold dilutions to achieve a final range of 50-0.016 μg/mL antibody after addition to 25 μL of media containing virus; final MOI 0.2) and incubated at 4° C. for 1 hr before the inoculum was added to the appropriate cell types (ARPE-19, HTR-svNeo, MRC5, or NHDF cells at 10,000 cells per well) for 2 hrs at 37° C., 5% CO₂. Following infection, the inoculum was removed and replaced with 100 μL complete DMEM/F-12 media before an overnight incubation at 37° C., 5% CO₂.

### Dissemination/Focus Forming Units Assay

ARPE-19 or NHDF cells were plates at 50,000 cells per well in a 24 well and infected the following day. TB40/E (MOI 0.01) and AD169RBAD^{rUL131} (MOI 0.1 & 0.01) was incubated with either 10 μg/mL or 0.5 μg/mL of antibody one hour prior to addition to cells. Following a 2 hr incubation at 37° C., 5% CO₂ the inoculum was removed, and cells were overlaid with 1% low melt temperature sea agarose overlay. The overlay was allowed to solidify at room temp and then 500 μL of media was added before cells were put back into the incubator. At 7 days post infection, the cells were scanned using the brightfield and GFP filters and then placed back into the tissue culture incubator until the assay was stopped by fixation and immunostaining on day 10 or 14 as indicated.

### Flow Cytometry Analysis

Surface staining was performed using 1% BSA in PBS on live cells. Intracellular staining was performed on fixed cells using 100 μL of CytoFix/CytoPerm (BD, #51-2090KZ) per 1E6 cells at 4° C. for 20 minutes as recommended then stained in the presence of 1% BSA (Akron Biotech, #AK8905-0100), 0.1% Saponin in PBS throughout the procedure. Briefly, cells were stained using 1 μg/mL primary antibody, washed and then stained with a goat-anti-mouse Fc AF647 secondary (1:500) (Thermo Fisher). Fluorescent data was collected on either an Intellicyte HTFC or an Attune NxT flow cytometer. The data was analyzed using Flow Jo software and graphed using Prism 8.4 software.

### Competition Assay

U373 gH/gL cells were trypsinized, fixed and resuspended in a permeabilization buffer (0.1% Saponin, 1% BSA in PBS) before being plated in a 96-well format at 50,000 cells per well and spun at 1800 rpm for 5 min at 4° C. 20 μg of each labeled antibody was conjugated to AF647 using an APEX labeling kit (Invitrogen, #A10475). The antibody was diluted to 0.5 μg/mL in perm buffer and mixed with increasing concentrations of unlabeled antibody (5-0.01 μg/mL final concentration). The labeled and unlabeled antibody was added to the U373 gH/gL cells and allowed incubated at 4° C. for 1 hr before unbound antibody was washed away and the cells were run on the flow cytometer. The percent MFI is calculated using the average MFI of labeled antibody binding in the presence of 5 μg (10×) of unlabeled PY102 as an irrelevant control.

### Pre-Post Attachment Neutralization Assay

MRC5 or ARPE-19 cells were seeded at 10,000 cells/well the day before infection and incubated at 37° C., 5% CO₂. The following day, BADrUL131 (MOI 0.2) was either added to cells directly or incubated with antibody (50 μg/mL) at 4° C. before being added to cells. Virus was incubated with cells at 37° C. (+/−antibody) for 2 hrs before being removed and replaced with complete cell culture media. Cells were incubated overnight and then fixed for immunostaining at 18 hpi using GFP and IE1-1 as a readout for infection. The percent infection was calculated using the irrelevant influenza antibodies PY102 or M2E10.

### Immunofluorescence Assay

Cells were fixed using 4% paraformaldehyde (PFA) for 20 minutes at room temp. Cells were then permeabilized using 0.3% Triton X-100 (Thermo Fisher, #HFH10) in PBS and stained with an affinity-purified, polyclonal rabbit anti-IE1-1/2 (0.7 ng/ml) primary and an anti-rabbit Alexa Fluor 647 (1:1000) secondary. To quantify total cells per well, Hoechst (Molecular Probes, #H3570) was diluted 1:100,000 in PBS and incubated with cells for 20 minutes at room temp. Virus neutralization was quantified using a Celigo cytometer (Nexelcom Bioscience). Percent infection was calculated using ((#IE1-1+)/(#Hoechst+))*100 events per well and normalized to the percent infection when an irrelevant, non-neutralizing, or no antibody was used. For 15G11 localization in late stage multinuclear bodies, ARPE-19 cells were infected with BADrUL131 reporter virus for 6 days before cells where fixed in 4% PFA and permeabilized with Triton-X as noted above. The cells were then blocked using 4% BSA/PBS before labeled CKAP4-AF488 and 15G11-AF647 were added at 5 μg/mL diluted in 4% BSA/PBS for 1 hr at RT. Unbound antibody was washed away and cells were stained with Hoechst (1:10,000) in PBS for 20 minutes at RT before being imaged.

### Immunoblotting Assay

Proteins from total cell lysates or proteins immunoprecipitated with the respective antibody were resolved on an SDS-polyacrylamide. The proteins were than transferred to a PVDF membrane using an electric current. The PVDF was blocked with 1% BSA (in PBS) for 1 hr at room temperature and subsequently incubated with a mouse or rabbit-derived antibody against a cellular or viral protein. The membrane was then incubated with an anti-mouse or anti-rabbit immunoglobulin conjugated to HRP and subjected to an enhanced chemiluminescence reagent. A radiographic film was used to visualize the polypeptides.

293Expi Transfection with Truncation Mutants of gH

gH mutants were cloned into pcDNA 3.1 and co-transfected with gL (1:1 ratio, 6 μg plasmid total) with lipofectamine 2000 (Thermo Fisher, #11668019) into 293Expi cells (7.5×10⁶). Cells were harvested 3 days post transfection and fixed before being incubated with α-gH antibodies (2 μg/mL) for 1 hr at RT. Cells were washed and then incubated in anti-mouse AF647 conjugated secondary (1:1000) in order to quantify binding. Transfection efficiency was determined using pMAX eGFP plasmid which was spiked in at 0.1 μg per well.

### gH AAAA Mutant Screen

5×10⁴ BHKs were plated in 24 well plate the day before transfection with 1500 ng of pcDNA plasmid at a 1:1 ratio of gH:gL using the manufacturer's protocol for Lipofectamine 2000. After 5 hrs the DNA/OPTI-MEM was removed and replaced with complete DMEM media. 48 hrs post transfection, the cells were fixed with 4% PFA and stained with labeled mouse monoclonal antibody (1 μg/mL) and Hoechst (1:100,000).

### Antibody Labeling

Each monoclonal antibody (20 μg) was labeled using the APEX Antibody Labeling Kit (Alexa Fluor™ 647, #A20186, Invitrogen) according to manufacturer's instructions.

### PepperPRINT DT

The sequence of gH (TB40/E strain) was elongated with neutral linkers (GSGSGSG) (SEQ ID NO:65) at the C- and N-terminus to avoid a truncation of peptides before being converted into 7, 10 and 13 amino acid peptides with overlaps of 6, 9 and 12 amino acids. After peptide synthesis, a thioether linkage between the C-terminal cysteine and an appropriately modified N-terminus allowed for cyclized resulting in conformational gH peptide microarray library containing 1,815 unique peptide printed in duplicate (3,360 peptide spots). Each microarray included 128 spots for HA control peptides flanking the array (YPYDVPDYAG) (SEQ ID NO:66).

Following pre-swelling and incubation with blocking buffer (Rockland MB-070, 30 min) the mAbs 1D11, 4E7, 9A12, 10F8, 13G1, and 15G11 were added at concentrations of 1, 10 and 100 μg/mL and allowed to incubate for 16 hr at 4° C. shaking at 140 rpm. Following a wash, each microarray copy was incubated with goat-anti-mouse DyLight 680 (0.2 μg/mL) for 45 minutes at room temperature and washed again before scan using a LI-COR Odyssey Imaging System and scanning intensities of 7/7 (red/green). The additional HA peptides lining the array were subsequently stained with mouse monoclonal anti-HA (clone 12CA5, 0.5 μg/mL) and used as an internal quality control to confirm assay quality and peptide microarray integrity. Quantification of spot intensities was based on a 16-bit grey scale tiff file and microarray image analysis was performed using the PepSlide® Analyzer.

### Immunoprecipitation Assay

Confluent U373-gHgL cells were harvested using trypsin (mM in PBS), counted and snap frozen on dry ice and stored at −80° C. Frozen cells were lysed using 1× NP-40 lysis buffer [NP-40, Leupeptin, Aprotinin, Phenylmethylsulfonyl fluoride (PMSF)] and incubated on ice for 20 minutes. Following the incubation, cell lysates were spun at 13,000 g for 5 minutes to remove cell membranes. For each IP, 5 μg of antibody was added to 1 mL of cell lysate (˜ 2 E6 cell lysates/IP) before 20 μL of Protein A was added. After 1 hr rocking at 4° C. the agarose beads were spun at 13,000 g for 5 minutes and washed 3 times with NET buffer before being resuspended with 50 μL of sample buffer [10% SDS, 1 M Tris pH 6.8, 50% glycerol, 600 mM DL-Dithiothreitol (DTT), bromophenol blue] and heated at 95° C. for 2 minutes. After a final spin to pellet beads, 35 μL was loaded into a 10% acrylamide gel and run using 35 V overnight. Proteins were transferred to a PVMF Membrane using 100 mAmps for 2 hrs. The resulting membrane was blocked in 10% milk and then incubated with anti-gL and anti-gH antibodies for 1 hr to detected specificity for HCMV glycoproteins. W6/32 was used as a negative control and 3 washes were performed before adding an anti-rabbit secondary antibody conjugated to HRP. Following a second round of washes, Immobilon Western Chemiluminescent HRP Substrate (Millipore, WBKLS 0500) was used to detect HRP activity on film.

### Virus Attachment

ARPE-19 cells were plated in glass bottom 12 well plates the day before infection. TB40/E UL32 e-GFP virus (MOI 0.1) was pre-incubated with α-BKV or 15G11 antibody at 10 μg/mL for 1 hr at 4° C. Infection occurred at 37° C. for 0-2 hrs before cells were washed using a citrate wash buffer (pH 3.2) for two minutes at RT. Cells were then washed with PBS and fixed in 4% PFA for 20 minutes at RT before being stained with Hoechst. Plates were read using a Cytation 3 Cell Imaging Multi-Mode Reader (BioTek, Winooski, VT) and images were acquired using brightfield and the 405 and 488 nm lasers.

### K_{D} Determination.

Biolayer interferometry assays were performed using the Octet RED instrument (ForteBio, Inc.) to determine the association (kₒₙ) and dissociation (k_{dis}) constants for each antibody. Purified monoclonal antibody was loaded onto the anti-mouse Fc IgG capture (AMC) biosensors using 5-fold dilutions (50-0.016 μg/mL in PBS) for 10 minutes. To determine the kₒₙ, the sensors were exposed to the recombinant HCMV pentamer (strain VR1814) consisting of gH, gL, UL128, UL130 and UL131A (Native Antigen, CMV-PENT-100) at a constant concentration of 100 μg/mL in PBS for 3 minutes. To determine the k_{dis}, dissociation was measured over the course of 3 minutes while the sensors were in PBS buffer. K_{D} values were calculated as a ratio of kₒₙ/k_{dis}. A binding model of 1:1 resulted in the best fit for each antibody.

### Statistical Analysis

The student's unpaired, two tailed t tests were performed using the GraphPad Prism software (La Jolla, CA). An asterisk identifies statistical significance of P<0.05 or less. The half maximal inhibitory concentration (IC50) values for each monoclonal were calculated using the 4-parameter non-linear regression analysis using GraphPad Prism and the standard deviation is displayed for all relevant figures.

### Example 2: Generation of a Panel of Broadly Neutralizing Human Monoclonal Antibodies Against HCMV gH/gL

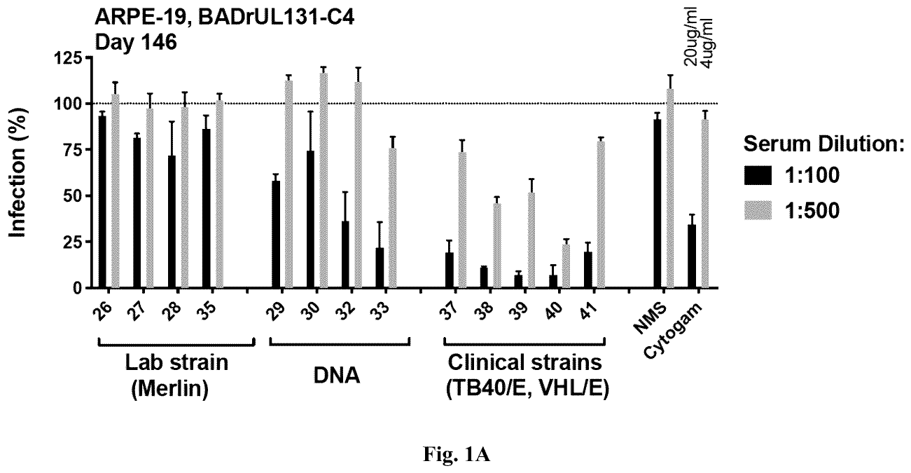

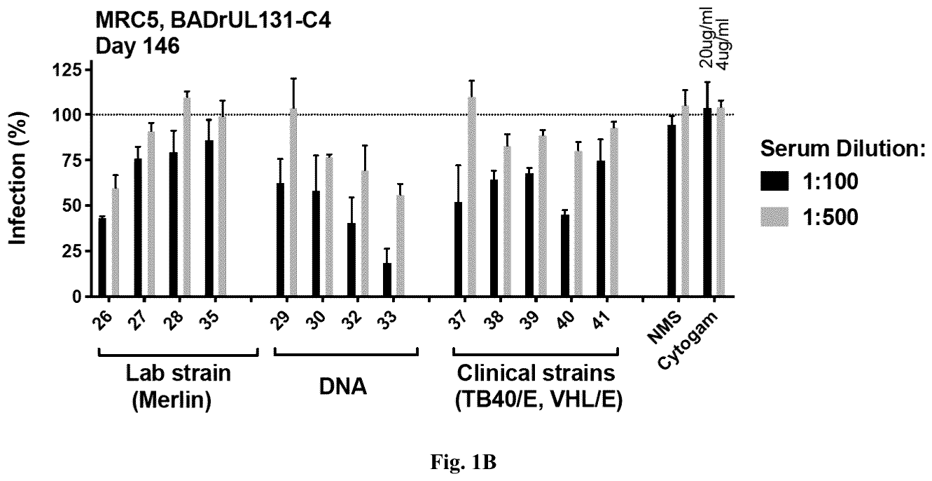

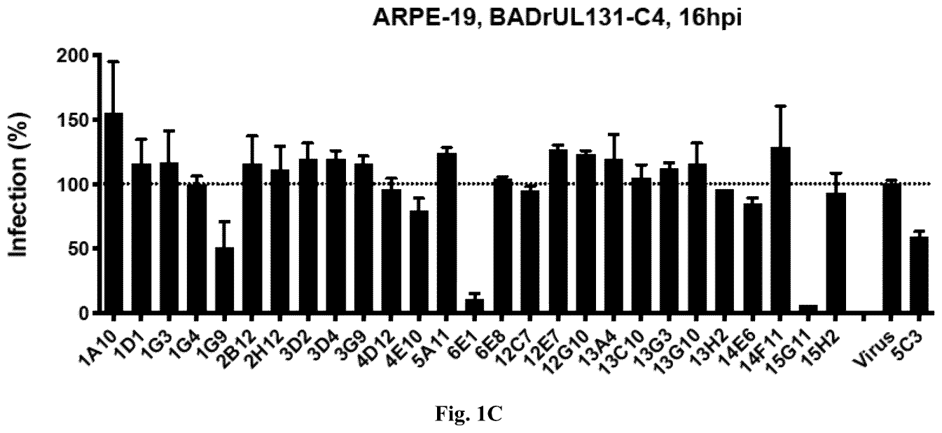

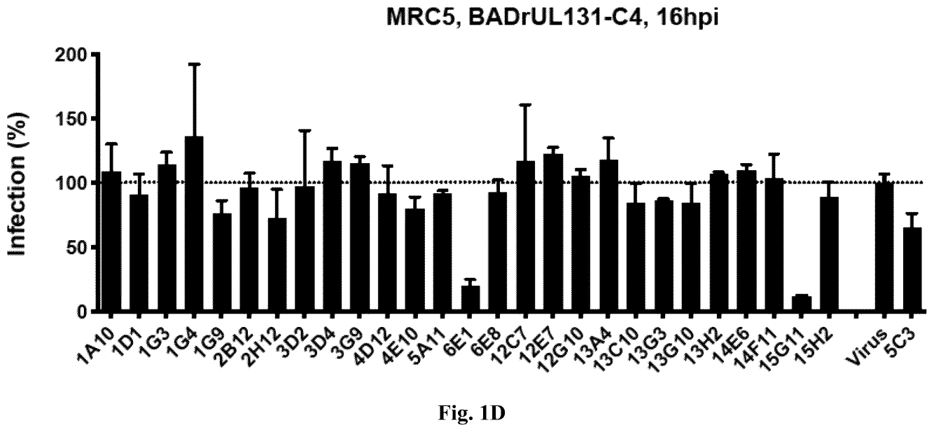

VelocImmune mice were vaccinated with either a lab adapted strain of HCMV (Merlin), a combination of clinical strains of HCMV (TB40E and VHL/E) or pcDNA plasmid expressing the gH/gL protein based on the TB40E WT coding sequencing. Following vaccination, the serum from immunized mice were assessed for neutralization capacity using a high-throughput neutralization assay (FIGS. 1A and 1B). Throughout the screening process the AD169 laboratory strain (denoted BADrUL131-C4) was used, which contains the UL131-UL128 open reading frame of the HCMV clinical strain TR and expresses the reporter eGFP.

Following immunization, sera from each mouse was diluted in complete media and pre-incubated with virus (MOI: 0.2) for one hour before being added to cells. Neutralization was examined at 18 hpi with Cytogam and normal mouse serum (NMS) used as positive and negative controls, respectively. The serum from mice immunized with gH/gL cDNA resulted in robust, dose-dependent neutralization of all mice and provided protection from the mismatched HCMV strain in both epithelial and fibroblast cell lines (FIGS. 1A and 1B). The vaccination induced a humoral response with increased neutralization in fibroblast-infected cells than epithelial cells. Vaccination with TB40/E and VHL/E produced antibodies more capable of neutralizing BADrUL131 infection in epithelial cells with >90% neutralization at both 1:100 and 1:500 dilution in some mice (FIGS. 1A and 1B). Mice immunized with Merlin displayed limited neutralization capacity when compared to the clinical strain or pcDNA vaccination groups. Based on the serum neutralization assay, the pcDNA vaccination strategy was superior to traditional virus-based vaccine strategies, capable of eliciting broadly neutralizing antibodies that block infection in both epithelial and fibroblast cell types.

To generate a panel of antibodies capable of inhibiting infection in epithelial and fibroblast cell types, three animals (mouse #29, 32, 33) were selected with the highest neutralizing capacity from the pcDNA vaccinated group to create hybridoma clones. Collectively, 4303 hybridoma clones were screened using high-throughput flow cytometry to detect binding to transiently expressed gH/gL on 293Expi cells. High binders were identified as clones with the enhanced mean fluorescence intensity (MFI) over untransfected cells. A heat map for each fusion was generated to summarize the MFI for each hybridoma clone.

The clones with the highest MFI were then used in a high-throughput neutralization assay using the reporter virus BADrUL131-C4 to identify the most potent neutralizers, representative data from Fusion 1 is shown (FIGS. 1C and 1D). Based on binding to 293Expi-gHgL and neutralization capacity, 24 clones were selected to be sequenced and isotyped. Of these clones, 12 unique IgG antibodies (11 neutralizers and 1 non-neutralizer) were expanded and purified for further characterization (Table 3).

TABLE 3
Hybridoma fusion identifies 12 unique antibodies which bind gH glycoprotein.
The genetic characteristics for each monoclonal antibody including isotype and CDR3
sequence is listed for fusions 1-3. IMGT/V-QUEST software was used to assign the germ line
reference for IGHV genes to determine the percent identity with germ line. The gene usage
for the panel is summarized below for the heavy chain variable V, D and J genes.

| | | | | | | | |
|---|---|---|---|---|---|---|---|
| | | | | V | | J | |
| | CDR3 | AA | V gene & | identity | J gene & | identity | D gene & |
| Antibody | length | Junction | allele | (%) | allele | (%) | allele |

Mouse 33

| | | | | | | | |
|---|---|---|---|---|---|---|---|
| 1G9 | 14 | CANHPNVL | IGHV5-51*01 | 96.88 | IGHJ6*02 | 85.48 | IGHD2-8*01 |
| | | MIFVQDFW | F | | F | | F |
| 6E1 | 15 | CARRGYNF | IGHV5-51*01 | 96.88 | IGHJ6*02 | 85.48 | IGHD5-18*01 |
| | | GYYYGMD | F | | F | | F |
| | | VW | | | | | |
| 15G11 | 10 | CARGGLGA | IGHV4- | 96.56 | IGHJ3*02 | 94 | IGHD7-27*01 |
| | | FDIW | 31*03F or | | F | | F |
| | | | IGHV4- | | | | |
| | | | 31*06F | | | | |

Mouse 32

| | | | | | | | |
|---|---|---|---|---|---|---|---|
| 1D11 | 15 | CAKSKHW | IGHV3-9*01 | 97.92 | IGHJ6*02 | 85.48 | IGHD4-17*01 |
| | | GDYYYTM | F | | F | | F |
| | | DVW | | | | | |
| 4E7 | 11 | CARDPNW | IGHV1-18*01 | 97.92 | IGHJ4*02 | 97.92 | IGHD1-1*01 |
| | | NFFDYW | F | | F | | F |
| 9A12 | 16 | CARSSGWY | IGHV3-48*03 | 95.83 | IGHJ6*02 | 90.32 | IGHD6-13*01 |
| | | RNYSYGM | F | | F | | F |
| | | DVW | | | | | |
| 10F8 | 10 | CAREAYSN | IGHV3- | 95.83 | IGHJ6*03 | 78.18 | IGHD4-11*01 |
| | | YGVW | 33*01 F or | | F | | ORF |
| | | | IGHV3- | | | | |
| | | | 33*06 F | | | | |
| 12H11 | 17 | CARLGHVQ | IGHV1-69*05 | 96.53 | IGHJ6*02 | 91.94 | IGHD3-16*01 |
| | | FGYYYYD | F | | F | | F |
| | | MDVW | | | | | |

Mouse 29

| | | | | | | | |
|---|---|---|---|---|---|---|---|
| 10H6 | 20 | CARRGNW | IGHV1-18*01 | 90.62 | IGHJ6*02 | 88.71 | IGHD1-1*01 |
| | | NPPYFYYR | F | | F | | F |
| | | YNGLDVW | | | | | |
| 11D3 | 13 | CARDSSGS | IGHV3-13*01 | 92.28 | IGHJ4*02 | 91.67 | IGHD3-22*01 |
| | | YSGFDYW | F | | F | | F |
| 13G1 | 15 | CARIDYSN | IGHV2-26*01 | 97.25 | IGHJ5*02 | 96.08 | IGHD4-11*01 |
| | | YIGNWFDP | F | | F | | ORF |
| | | W | | | | | |
| 14E1 | 10 | CARDHGFF | IGHV1-18*01 | 97.92 | IGHJ4*02 | 93.75 | IGHD1-26*01 |
| | | FDYW | F | | F | | F |

Fusion 1 resulted in three unique antibodies and fusion 2 and fusion 3 resulted in five antibodies each. Sequencing of the heavy chain from each hybridoma clone revealed diverse CDR3 lengths ranging from 10⁻²⁰ aa and found the IGHJ6*02 gene to be predominant (50%) across the panel. An unrooted phylogenetic tree was created to determine if vaccination with gH/gL DNA elicits a similar response in individual animals (identify shared motifs, AA junctions, commonly used alleles etc). Isotyping of the panel shows a strong skewing toward IgG2a antibody production (11/12 clones) and only one IgG2b clone were identified. Serial vaccination with gH/gL cDNA in three animals elicited a broad, strongly neutralizing immune response, consisting primarily of IgG2a antibodies with diverse CDR3 regions.

### Example 3: HCMV-Neutralizing Antibodies are Broadly Neutralizing and Cross-Protective

The monoclonal antibodies were next evaluated for their ability to limit infection in diverse cell types important for virus entry and spread during natural infection in vivo.

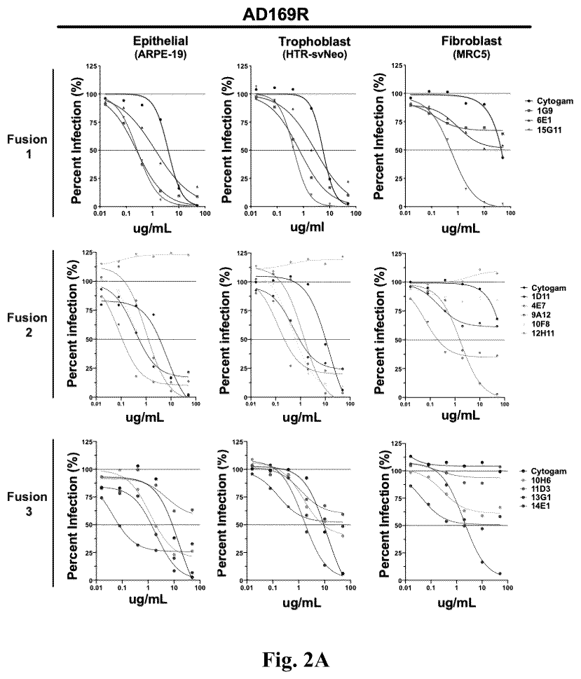

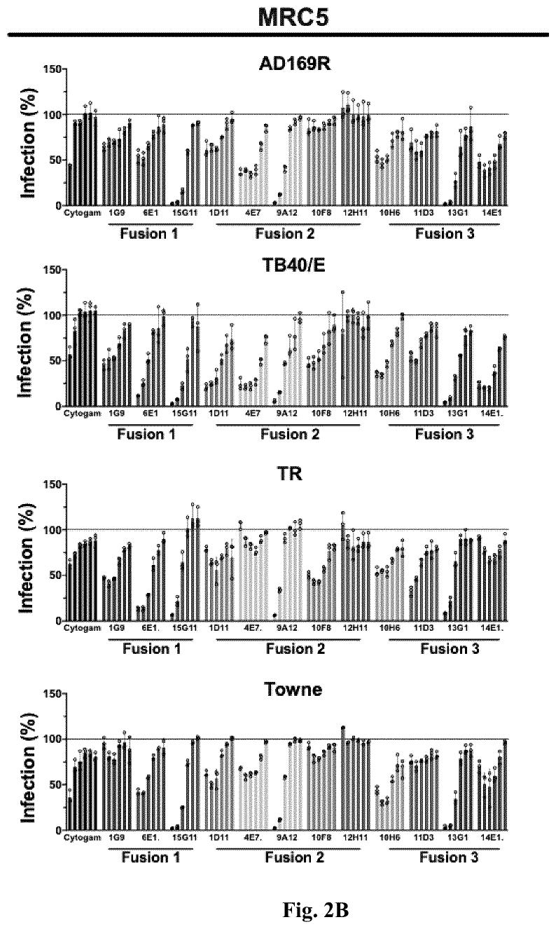

For this, the BADrUL131-C4 viral strain (MOI 0.2) was pre-incubated with purified monoclonal antibodies using 5-fold dilutions starting at 50 μg/mL for one hour before adding to epithelial, endothelial or fibroblast cell lines to generate neutralization curves. The neutralization curves for each antibody demonstrated similar trends between epithelial cell line ARPE-19 and the placental tissue derived trophoblast cell line HTR-8/SVneo (FIG. 2A). The FDA-approved polyclonal immunoglobulin marketed as Cytogam was used as a positive control and as previously reported Cytogam is less efficacious in fibroblast infections because it is enriched in antibodies directed against the UL128-131A envelope proteins rather than gO.

The non-neutralizing antibody 12H11 from Fusion 2 did not provide protection from HCMV in the epithelial, endothelial or fibroblast cell lines tested however, neutralization curves generated for the mAb panel using PMA-differentiated, THP-1 macrophages using both the vaccination matched TB40/E WT and BADrUL131-C4 viral strains (FIGS. 2C and 2D) show that 12H11 was able to neutralize ˜50% of the TB40/E WT infection in THP-1 macrophages. The most effective antibody from each fusion (15G11, 9A12 and 13G1) consistently neutralize >90% of the infection at the highest concentrations regardless of cell type indicating that they bind to a region of gH/gL vital for entry. The majority of antibodies were markedly more effective in epithelial or trophoblast cells than fibroblast indicating that targeting gH/gL has a greater impact for entry into epithelial cells. The half maximal inhibitory concentration (IC50) (Tables 4, 5, and 6) shows that the IC50s of 15G11, 9A12 and 13G1 significantly limited virus infection of all strains and in all cell types tested (IC50: 0.09-6.96 μg/mL). As expected, CytoGam® was ineffective at blocking virus entry and in some case the IC50 could not be determined in the context of fibroblast infection.

In summary, a group of mAbs were identified that broadly block HCMV infection.

TABLE 4
Ability of isolated antibodies to limit virus infection
(IC50 values) for indicated cell lines and antibodies.

| Mouse | | | IC₅₀(ARPE-19) | | IC₅₀(HTR-svNeo) | |
|---|---|---|---|---|---|---|
| ID | Fusion | Antibody | TB40/E | AD169R | TB40/E | AD169R |
| — | — | Cytogam | 0.45 | 3.92 | 3.88 | 5.55 |
| | 1 | 1G9 | 0.04 | 0.25 | 0.56 | 0.70 |
| Mouse | 1 | 6E1 | 0.19 | 1.30 | 2.12 | 2.81 |
| 33 | | | | | | |
| | 1 | 15G11 | 0.09 | 0.24 | 0.41 | 0.44 |
| | 2 | 1D11 | 0.43 | 0.68 | 0.43 | 1.48 |
| | 2 | 4E7 | 0.26 | 0.12 | 0.25 | 0.35 |
| Mouse | 2 | 9A12 | 1.10 | 1.28 | 0.72 | ~1.802 |
| 32 | | | | | | |
| | 2 | 10F8 | 0.50 | 0.80 | 0.38 | 0.69 |
| | 2 | 12H11 | >50 | >50 | >50 | >50 |
| | 3 | 10H6 | 7.48 | 2.63 | 0.65 | 15.03 |
| Mouse | 3 | 11D3 | 7.09 | >50 | 0.51 | >50 |
| 29 | | | | | | |
| | 3 | 13G1 | 1.01 | 1.03 | 1.12 | 1.82 |
| | 3 | 14E1 | 3.06 | 0.11 | >50 | 19.21 |

TABLE 5
Ability of isolated antibodies to limit virus infection
(IC50 values) for indicated cell lines and antibodies.

| | | | IC₅₀(MRC5/NHDF) | | | | |
|---|---|---|---|---|---|---|---|
| Mouse ID | Fusion | Antibody | TB40/E | AD169R | AD169 | TR | Towne |
| — | — | Cytogam | >50 | 41.70 | >50 | >50 | 31.68 |
| | 1 | 1G9 | 12.04 | >50 | 16.88 | 5.53 | >50 |
| Mouse 33 | 1 | 6E1 | 2.33 | 36.61 | 6.94 | 0.63 | 8.60 |
| | 1 | 15G11 | 0.50 | 0.51 | 0.31 | 3.51 | 0.87 |
| | 2 | 1D11 | 0.44 | >50 | 2.73 | >50 | 40.53 |
| | 2 | 4E7 | 0.09 | 0.75 | 0.20 | >50 | >50 |
| Mouse 32 | 2 | 9A12 | 1.09 | 1.52 | 0.77 | 6.96 | 2.53 |
| | 2 | 10F8 | 11.05 | >50 | 6.90 | 5.13 | >50 |
| | 2 | 12H11 | >50 | >50 | >50 | >50 | >50 |
| | 3 | 10H6 | 0.30 | 17.64 | 0.70 | 39.25 | 0.91 |
| Mouse 29 | 3 | 11D3 | 1.94 | >50 | 0.01 | 7.37 | >50 |
| | 3 | 13G1 | 0.89 | 0.58 | 15.92 | 3.26 | 1.09 |
| | 3 | 14E1 | 0.89 | 1.78 | 0.91 | >50 | >50 |

TABLE 6
Ability of isolated antibodies to limit virus infection
(IC50 values) for indicated cell lines and antibodies.

| | | | IC₅₀(THP-1) | |
|---|---|---|---|---|
| Mouse ID | Fusion | Antibody | TB40/E | AD169R |
| — | — | Cytogam | 1.73 | 20.49 |
| | 1 | 1G9 | 0.13 | 0.23 |
| Mouse 33 | 1 | 6E1 | 0.07 | 0.21 |
| | 1 | 15G11 | 0.39 | 0.01 |
| | 2 | 1D11 | 0.24 | 0.35 |
| | 2 | 4E7 | 0.04 | 0.04 |
| Mouse 32 | 2 | 9A12 | 0.00 | 0.08 |
| | 2 | 10F8 | 0.02 | 0.25 |
| | 2 | 12H11 | 0.05 | 0.65 |
| | 3 | 10H6 | 2.90 | 0.80 |
| Mouse 29 | 3 | 11D3 | 26.02 | 1.36 |
| | 3 | 13G1 | 0.02 | 0.03 |
| | 3 | 14E1 | 0.00 | 0.02 |

Next, the ability of the mAbs to neutralize HCMV strains TR and Towne was evaluated (FIG. 2B). This larger panel of HCMV viruses represents strains isolated in North America and Europe, as well as isolates collected between 1956 and 1999, with a gH/gL sequence identity >96.6%. Even with subtle changes in the gH/gL sequence across strains, several antibodies demonstrated reduced neutralization capacity when pre-incubated with different HCMV strains. In fact, 4E7 was unable to neutralize TR specifically while 10F8 was able to neutralize TB40 and TR but loses the ability to neutralize BADrUL131-C4 and Towne. The broadly neutralizing antibodies 15G11, 9A12 and 13G1 again consistently neutralized infection in MRC5 across all strains (FIG. 2B).

In addition to the standard IC50, the maximum neutralization at a high and low concentration (50 and 2 μg/mL respectively) of antibody was displayed using a heat map. The requirement for high concentrations of CytoGam® to neutralize infection is blatantly clear as CytoGam® can only block ˜20-40% infection at 2 μg/mL as compared to the panel of antibodies when used at 2 μg/mL and several monoclonals are able to neutralize >60% of the infection.

In short, these newly developed monoclonal antibodies better control infection than CytoGam® in fibroblast cell types both at high and at low concentrations.

### Example 4: Isolated Anti-HCMV Antibodies Limit Viral Dissemination

Next, a focus forming unit (FFU) assay was optimized to examine the antibodies ability to block viral replication and dissemination over multiple infection cycles. Using a Celigo Imaging Cytometer, the infection in live cells was followed by collecting brightfield and GFP images along a time course after infection with BADrUL131 reporter virus. Once the assay was terminated at Day 10 or 14, the cells were fixed and performed immunostaining to quantify total cells by Hoechst or infection using IE1-1 as readout for infection. These images can be overlaid to show the formation and spread of HCMV multinuclear bodies over time. The number of FFUs was quantified per well, using 10,000 μm² as a cutoff for plaque area. Briefly, ARPE-19 and NHDF cells were infected with BADrUL131 (MOI 0.01 and MOI 0.001 respectively) in the presence of antibody (10 or 0.5 μg/mL) for 1 hr before both virus and antibody were removed and cells were overlaid with 1% Seaplaque Agarose to limit cell-free infection. Images were taken on day 7 post infection and the relative number of plaques per well was calculated compared to wells that received an irrelevant control antibody M2E10. Cytogam was used as a positive control in each of the FFU assays. As shown previously in the high-throughput neutralization assay (HTN), the monoclonal antibodies are more effective in limiting infection of epithelial cells than fibroblast cells.

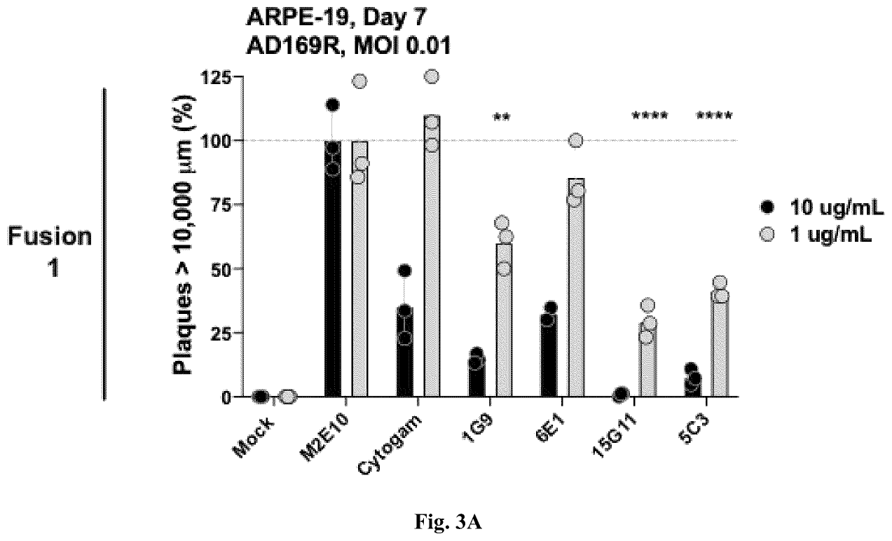

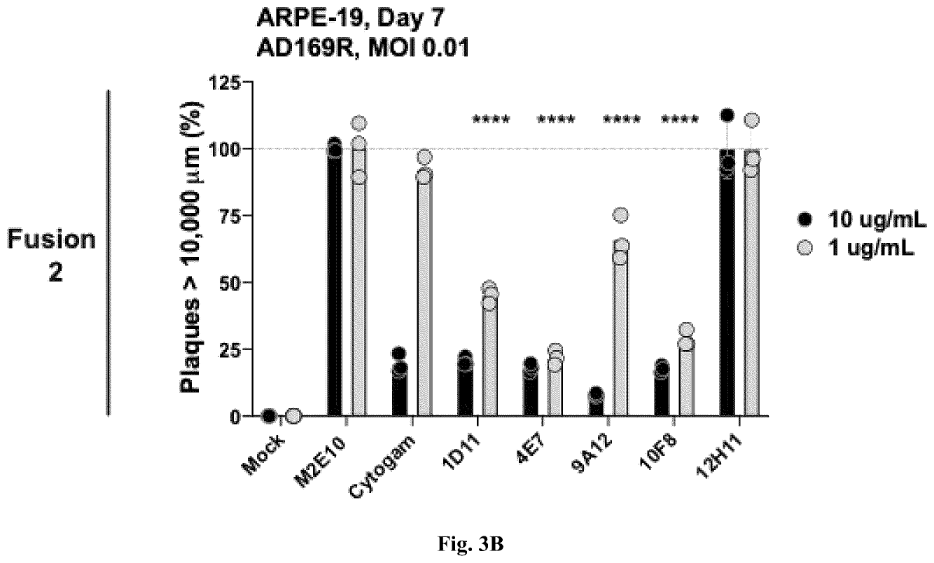

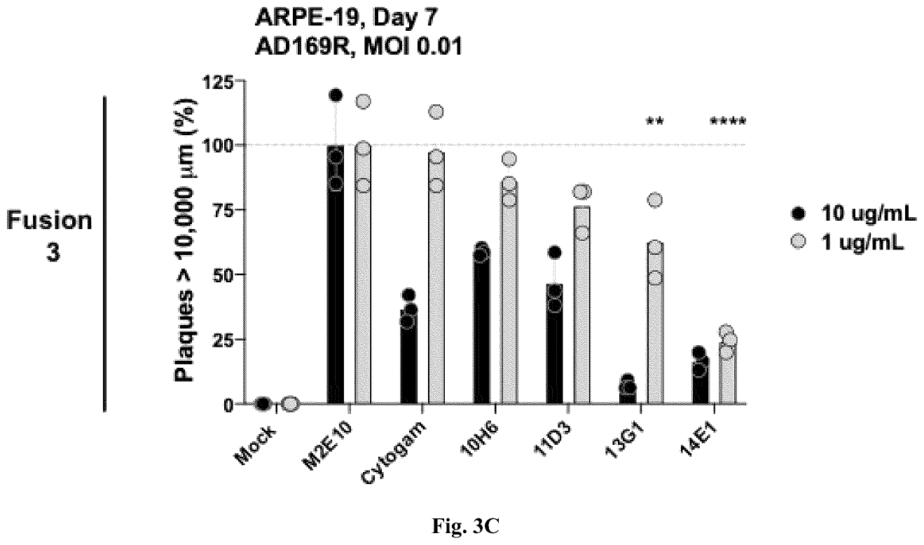

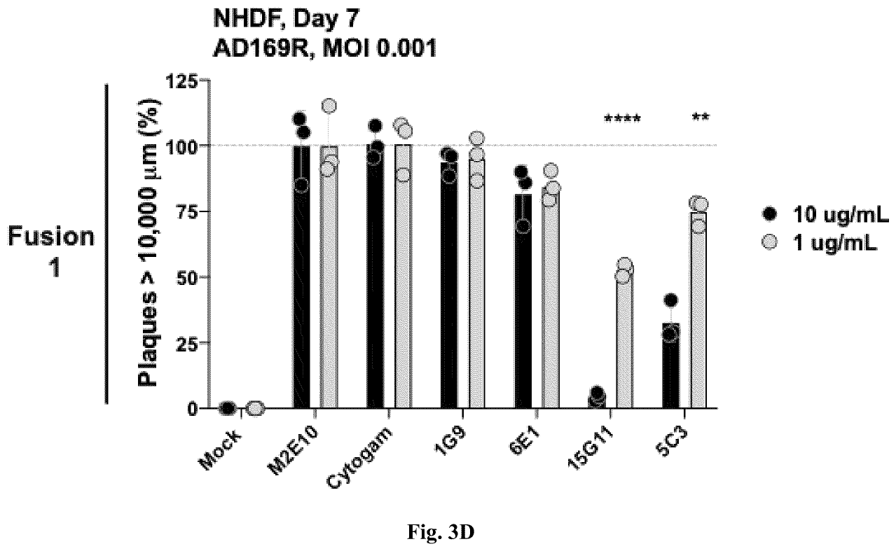

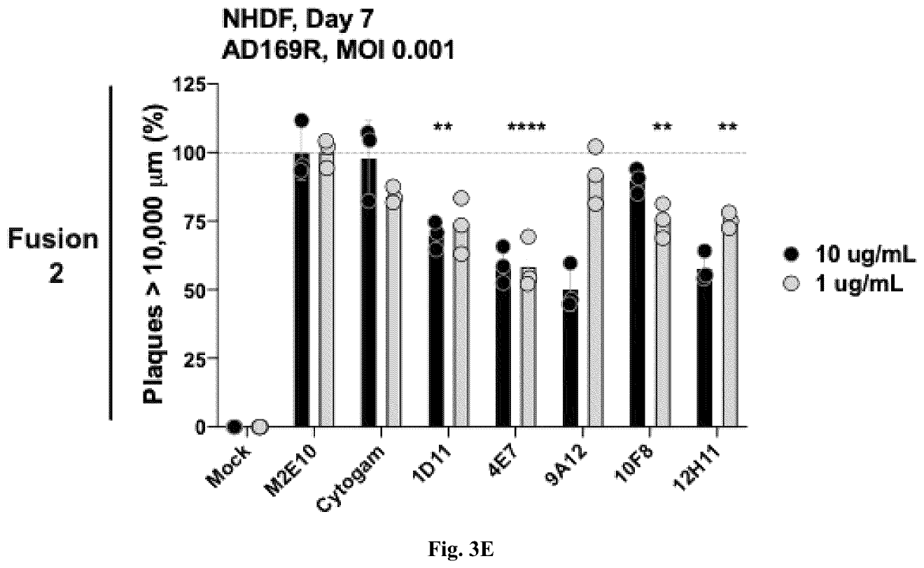

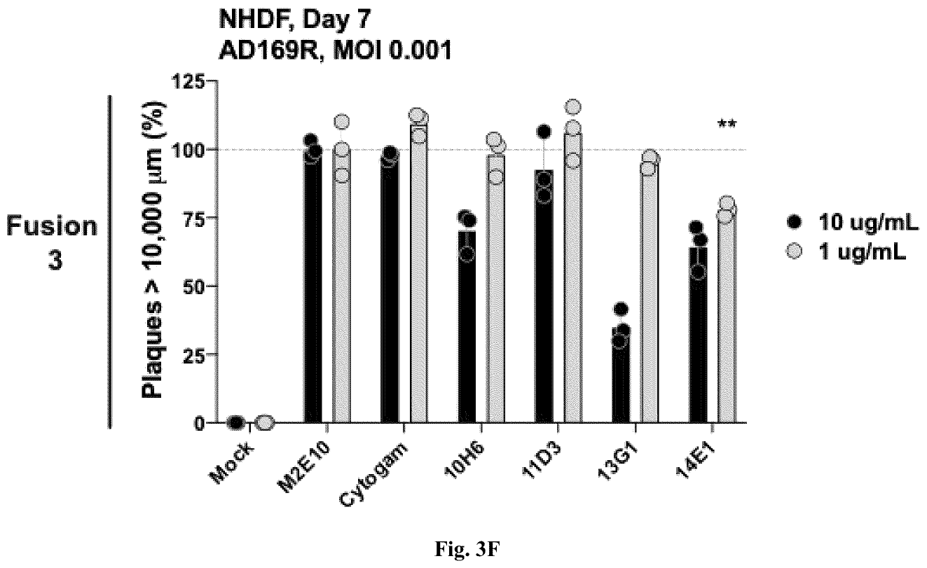

With the exception of the non-neutralizing antibody 12H11, each of the antibodies limited viral dissemination in a dose dependent manner and many outperformed the currently approved polyclonal antibody therapy CytoGam® at both concentrations tested. Interestingly, 4E7, 10F8, and 14E1 were effective when diluted 20-fold to a final concentration of 0.5 μg/mL (FIG. 3). Only a select few antibodies were able to reach 50% reduction in FFU value compared to M2E10 by day 7 in NHDF fibroblast cells. This included 15G11, 9A12 and 13G1. Of note, 4E7 nearly reached the cutoff of 50% and 12H11, the non-neutralizer seems to have a minimal impact on viral dissemination in NHDF using the BADrUL131 strain that was previously not observed in the HTN assay. This data represents the number of plaques >10,000 μm² but the same trends can be seen when examining at the total number of infected cells per well by either GFP or IE1-1 straining. FFU assays using the TB40E WT strain have also been performed for Fusion 2 antibodies using ARPE-19 and the same trends in neutralization are observed (data not shown). Even after 14 days post infection, the wells that received virus incubated with monoclonal antibodies from fusion 2 have significantly less infected cells per well than Cytogam or M2E10, indicating that these antibodies are quite effective at limiting HCMV dissemination.

### Example 5: Monoclonal Antibodies Target Multiple HCMV Envelope Protein Complexes

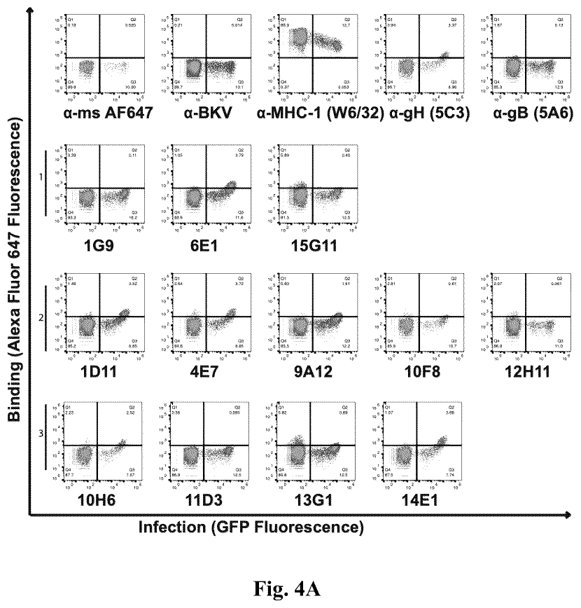

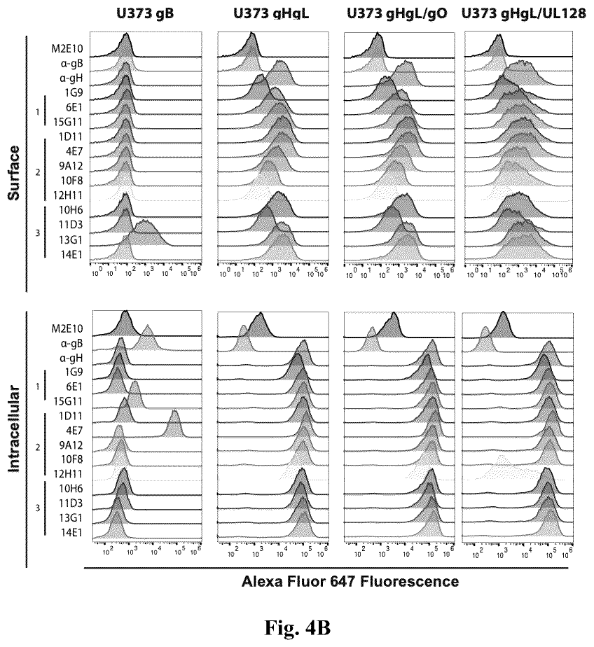

To confirm that the antibodies were able to bind to glycoprotein complexes generated during natural infection, the binding to in vitro infected ARPE-19 cells at 6 days post infection was assessed with BADrUL131-C4 by flow cytometry. The percent infection was calculated based on GFP expression (x axis) and the percent binding of each antibody was quantified using an anti-mouse Fc antibody conjugated to AF647 fluorophore (y axis). The contour plots for each antibody revealed binding to mock and day 6 post infection ARPE-19 cells. While GFP signal was high by day 6 post infection in almost all cells, the expression of late viral proteins like gH/gL was not absolute. However, binding could be detected, typically in cells with the highest GFP fluorescence. 12H11 has limited binding to infected cells and 4E7 seems to bind both mock and infected ARPE-19 cells. (FIG. 4A).

To confirm specificity for the gH/gL dimer, the trimer or, the pentamer of HCMV, an astrocytoma cell line (U373) was used, which constitutively expresses either gB, gH/gL, gH/gL/gO or gH/gL/UL128 (FIG. 4B). gH/gL/UL128 was used as a surrogate for the complete pentamer complex, creating a conformation similar to the complete pentamer. Each of the antibodies was incubated with either live or fixed/permeabilized cells and then the percent of binding and mean fluorescence intensity (MFI) for each cell line was assessed by flow cytometry (FIG. 4B). The previously characterized gH/gL antibody 5C3 (Gardner, T. J., et al., Functional screening for anti-CMV biologics identifies a broadly neutralizing epitope of an essential envelope protein. Nat Commun, 2016. 7: p. 13627) and gB antibody 5A6 (Stein, K. R., et al., CD46 facilitates entry and dissemination of human cytomegalovirus. Nat Commun, 2019. 10 (1): p. 2699.) were used as positive controls for binding. An influenza specific antibody (M2E10) was used as a negative control.

Although most of the antibodies were able to bind to gH/gL efficiently regardless of complex formation, the non-neutralizer (12H11) again shows reduced binding to the surface of gHgL expressing cells and significantly less binding to cells expressing the gH/gL/UL128 complex. Most interestingly, the 4E7 antibody from fusion 2 only bound to U373-gB cells when permeabilized, indicating it may be able to bind an additional host-derived, intracellular protein(s). The 13G1 antibody seems to bind non-specifically to the surface of U373-gB cells yet does not significantly bind the same cell type when fixed and permeabilized. The non-neutralizing antibody 12H11 also showed significantly reduced affinity for the gH/gL/UL128 complex but was able to sufficiently bind dimer and trimer indicating it has a unique binding site that is not involved in the entry process. This antibody, although a non-neutralizer, is useful as a diagnostic or reporter antibody which can bind tightly to gH/gL but does not interfere with the ability of gH/gL to mediate HCMV binding and/or fusion. Immunoprecipitation using U373-gB cell lysate in combination with 13G1, 15G1 was attempted to identify additional binding proteins but no clear bands were seen.

### Example 6: Anti-HCMV Antibodies do not Block Virus Attachment

To visualize HCMV virion attachment and entry, a recombinant HCMV strain was used that expresses eGFP fused to the C terminus of the tegument protein pUL32. The tegument protein is tightly associated with the capsid and allows for visualization of virus entry by immunofluorescence. TB40/E UL32-eGFP virus was incubated with either an irrelevant antibody (α-BKV) or 15G11 at 10 μg/mL for 1 hr at 4° C. to allow for immune complex formation before being added to ARPE-19 epithelial cells in a glass bottom plate. Cells were incubated with the virus/antibody for either 0 or 120 minutes before being washed with citrate buffer (pH 3.2) for two minutes and fixed with PFA. The presence of the neutralizing antibody 15G11 did not significantly change the number of virions bound to the cells, indicating that the gH antibody works to neutralize HCMV infection at a post attachment step.

### Example 7: Anti-HCMV Antibodies Bind Two Distinct Regions of gH/gL

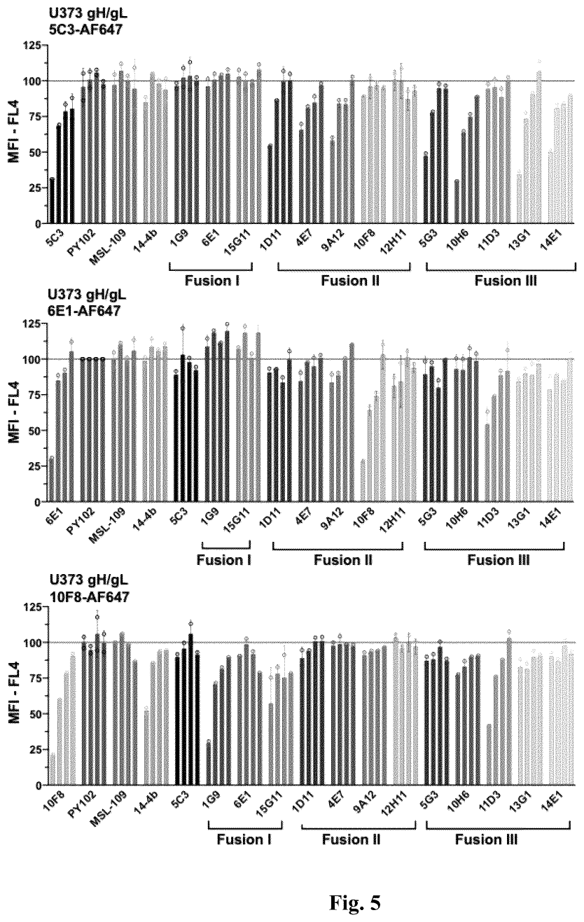

In order to determine which region of gH/gL the mAbs bind, a series of competition assays were performed (FIG. 5). A fixed amount of Alexa Fluor 647 labeled antibody (0.5 μg/mL) was mixed with increasing amounts of unlabeled antibody (0.1, 0.5, 1 or 5 μg/mL) before being added to U373-gHgL cells for 1 hr. The binding epitope of 5C3 was previously identified and is contained within a highly conserved region of gH (residues 481-HTTERREIFI-490) (SEQ ID NO:67).

The competition data indicates that seven of the new antibodies bind to a region close enough to the 5C3 epitope to compete away labeled 5C3. Similar experiments were performed labeling additional antibodies. A second region of gH was identified that is bound by 10F8. 10F8 can be displaced by increasing concentrations of 14-4b. a known gH antibody that recognizes a discontinuous epitope likely located around the membrane proximal ectodomain of gH. In addition to 10F8, 1G9, 15G11, and 11D3 all seem to compete for binding in this second region. An excess of 6E1 was not sufficient to reduce fluorescent signal when 10F8 was labeled but the reverse is true, which indicates either differences in binding affinity or slight differences in the antibody footprint on the surface of gH/gL.

To examine this further, 293 Expi cells were transfected with wildtype gH, wildtype gL, cotransfected with gH and gL (gH+gL), transfected with a single plasmid expressing both gH and gL (gH/gL) or co-transfected a variety of gH mutants and gL. Three days post transfection the cells were harvested and stained with unlabeled primary at 0.5 μg/mL using the antibodies indicated. Transfection efficiency was determined by measuring GFP expression on day 3 and binding was determined by percent Alexa Fluor 647 positive cells using a conjugated secondary. The difference in binding patterns between 10F8 and 6E1 indicates that 6E1 does bind a different epitope than 10F8 which is more sensitive to conformational changes in gH and cannot bind gH monomer. The binding profile for 1G9 is most similar to 10F8. Although the ratios of gH, gL and gO plasmid were 1:1:1 it is not certain how efficiently the trimer was assembled following transfection. Antibodies 5C3, 6E1 and 9A12 bound to cells transfected with gH alone or gH/gL/gO but antibodies 1G9 and 10F8 had reduced binding to gH monomer or gH/gL/gO trimer. To discern minute differences in antibody binding biolayer interferometry was used, immobilizing each of the purified monoclonal antibodies (50-0.016 μg/mL) onto anti-mouse IgG Fc capture (AMC) biosensors and exposing the sensors to recombinant pentamer (100 μg/mL). The binding constants ranged widely (1.41-64.60 nM), with 15G11 and 14E1 being the strongest and weakest binders respectively (Table 7).

TABLE 7
Binding data for indicated antibodies.

| | K_{D} (nM) | kₒₙ(1/Ms) | k_{dis}(1/s) | Full X\hat{ }2 | Full R\hat{ }2 |
|---|---|---|---|---|---|
| 5C3 | 7.64 | 1.42E+04 | 1.09E−04 | 0.4975 | 0.9993 |
| 1G9 | 19.20 | 2.29E+04 | 4.40E−04 | 0.5376 | 0.9977 |
| 6E1 | 16.90 | 5.40E+04 | 9.11E−04 | 1.9191 | 0.9944 |
| 15G11 | 2.28 | 3.82E+04 | 8.69E−05 | 1.6738 | 0.9986 |
| 1D11 | 5.66 | 8.23E+03 | 4.66E−05 | 0.0561 | 0.9999 |
| 4E7 | 8.08 | 2.13E+04 | 1.72E−04 | 0.2226 | 0.9997 |
| 9A12 | 3.47 | 2.48E+04 | 8.60E−05 | 0.2753 | 0.9992 |
| 10F8 | 4.91 | 2.59E+04 | 1.27E−04 | 0.2763 | 0.9997 |
| 12H11 | 15.90 | 2.44E+04 | 3.88E−04 | 0.1464 | 0.9854 |
| 10H6 | 13.00 | 7.57E+03 | 9.82E−05 | 0.1841 | 0.9993 |
| 11D3 | 1.41 | 1.17E+04 | 1.65E−05 | 0.3144 | 0.9996 |
| 13G1 | 5.05 | 1.29E+04 | 6.51E−05 | 0.113 | 0.9998 |
| 14E1 | 64.60 | 2.44E+04 | 1.58E−03 | 0.9419 | 0.9923 |

### Example 8: Epitope Mapping of Several Anti-HCMV Antibodies Using PEPperCHIP

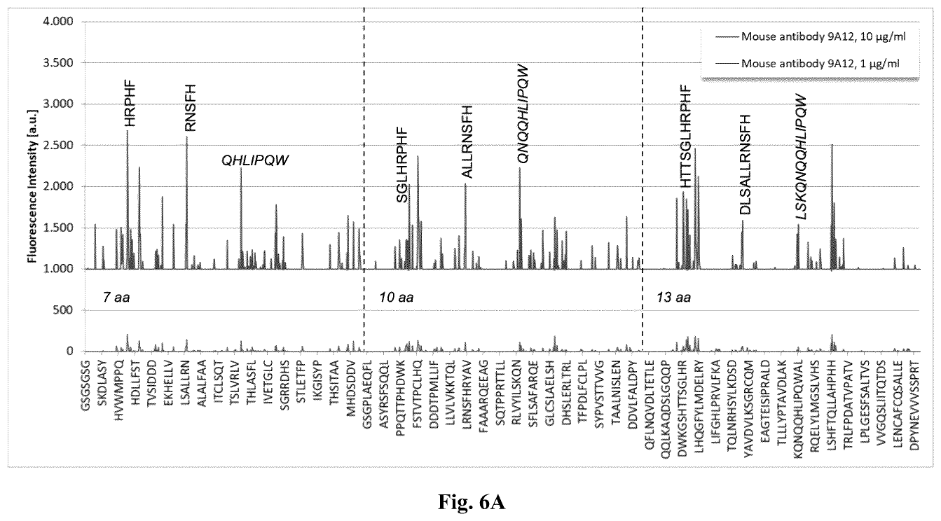

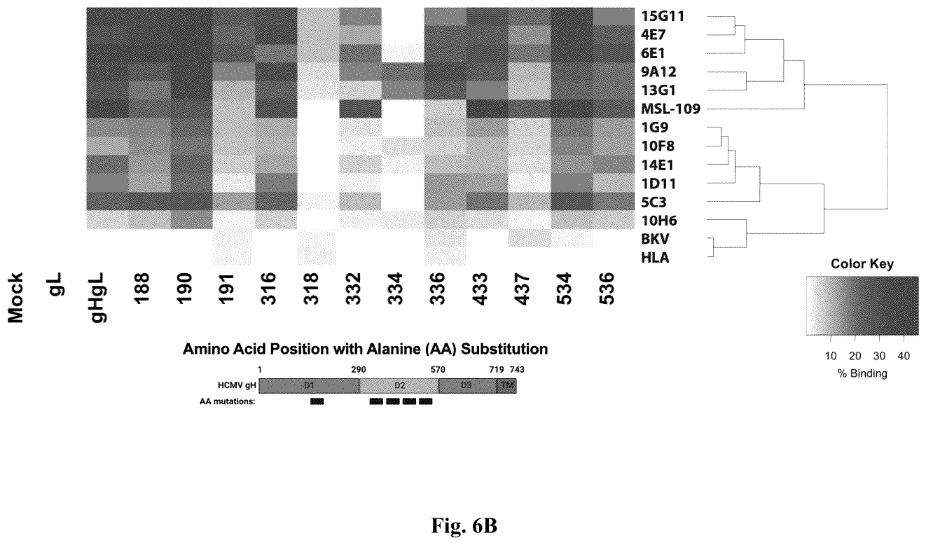

To further define the gH epitopes of several antibodies, a cyclic peptide microarray platform (PEPperCHIP) was used to identify the 5C3 and 10C10 epitopes in Gardner, et al. 2016. This assay utilizes an overlapping gH peptide library consisting of peptides of 7, 10 and 13 aa in length and is used to identify which region(s) of gH are bound by an individual antibody. The results for one representative microarray are shown in FIG. 6A. The 1D11 antibody bound two peptides within domain 2 of gH, similarly to 5C3 but localized to the opposite face of the glycoprotein and shared several amino acids, 525 SGRR 528. The minimal peptide 187HRPHF191, found in domain 1 of gH was consistently bound by 9A12, 10F8 and 15G11. In addition to this peptide 9A12 and 15G11 also bounding strongly to 426 LSKQNQQHLIPQW 438 located in domain 2. The PEPperCHIP results predicted binding to 2-3 distinct regions of gH, one of which lies within the alpha helix-rich domain (near the cleft) which has been shown to be susceptible to antibody neutralization in prior publications.

To confirm the identity of these epitopes, alanine mutations were designed at each of the predicted binding sites and performed transfections with wildtype or mutant gH cDNA in BHK cells. The percent binding relative to wildtype is summarized in a heat map (FIG. 6B) for each antibody except 11D3 and the non-neutralizing antibody 12H11. Several attempts were made to label these antibodies with AF647 but low labelling efficiency was observed each time. In addition to the antibody panel, human gH antibody MSL-109 was used as a positive control and two antibodies (anti-BKV and W6/32 an antibody that binds MHC-I) as negative controls. A total of 12 alanine mutants covering 5 regions and spanning domain 1 and domain 2 of gH were tested. Mutation of gH at 318 abbrogated binding across the entire panel and mutation at gH 334 disrupted binding for all antibodies except 9A12 and 13G1.

### Example 9: Anti-HCMV Antibodies can be Used in Combination with Ganciclovir

Ganciclovir is an acyclovir analog commonly used to treat HCMV retinitis in patients with immunosuppression, typically from HIV, AIDS or due to organ transplant. This broadly neutralizing synthetic nucleoside analogue of 2′-deoxyguanosine is incorporated into viral DNA during replication, leading to reduced infectious virions and reduced viral spread.

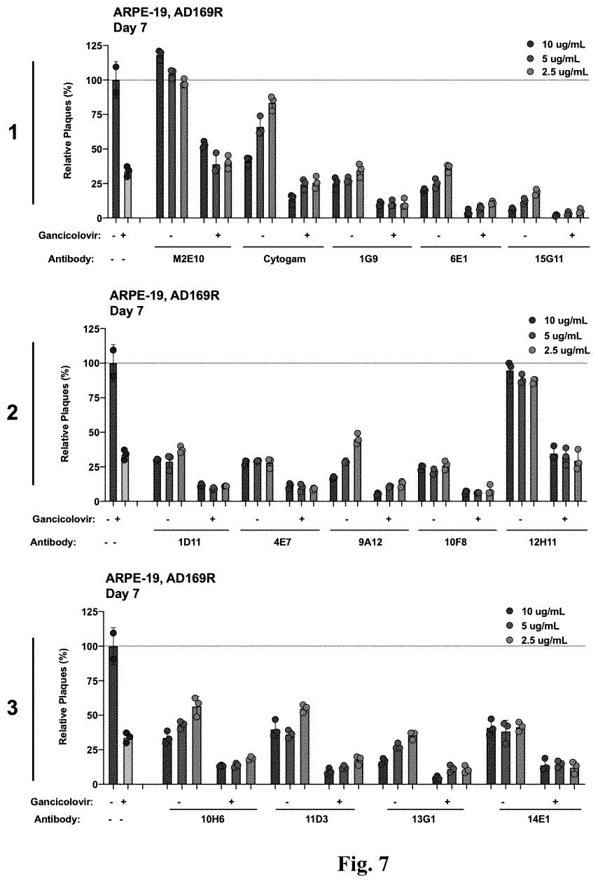

To test efficacy of the gH antibodies in combination with ganciclovir, the FFU assay was performed in ARPE-19 cells. A concentration of ganciclovir below EC50 was used (2.5 μM). Three concentrations of antibody were tested for each antibody in the panel and all conditions were performed in triplicate. M2E10 and CytoGam® were used as negative and positive controls respectively and the relative number of plaques was calculated based on cells which received virus alone without any antibody (black bar for each graph). Each 24 well plate also included wells that received ganciclovir alone as indicated by the grey bars on each graph. Although the results were striking when imaged at Day 7 post infection, the significant reduction in plaques number is still evident out to 12 days post infection (FIG. 7). For example, 15G11 when combined with ganciclovir resulted in a 95% reduction in plaques at 10 μg/mL on Day 12. The non-neutralizing antibody 12H11 served as good internal control and no significant reduction in infection was seen. By day 12, the M2E10 condition has large plaques that have begun to merge together making quantification difficult and this results in a reduction in relative plaques. However, the gH antibodies from fusion 1-3 outperform CytoGam® and work in combination with ganciclovir indicating that they are be useful in combination in the clinic to treat patients with severe immune deficiencies.

### Example 10: Fully Human CMV Neutralizing Antibodies Limit Virus Infection and Dissemination in an Animal Model

VelocImmune® animals, which were used for the production of antibodies 15G11, 9A12, 13G1 & 14E1, produce chimeric antibodies with human variable genes and mouse constant genes (see Example 2). Monoclonal antibodies 15G11, 9A12, 13G1 & 14E1 were selected for conversion to fully human antibodies. For this, the mouse Fc region of these mAbs was replaced with the human IgG1 Fc region.

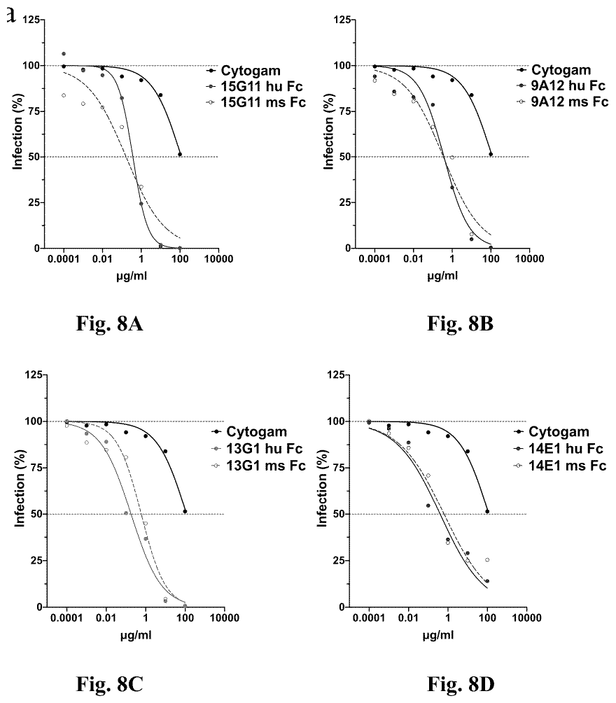

To ensure that the Fc domain did not impact their neutralization capacity, a neutralization study was performed. The completely human versions of 13G1 (FIG. 8C) and 14E1 (FIG. 8D) showed improved IC50 values of infection as compared to their chimeric counterparts. The IC50 value of infection for the fully human version of 9A12 remained unchanged as compared to the version of the antibody with mouse constant regions (FIG. 8B). The IC₅₀ of infection for 15G11 increased from 0.17 μg/mL to 0.37 μg/mL after replacing the murine constant regions with human constant regions (FIG. 8A). These findings show antibodies targeting gB and gH/gL-complexes have a similar impact on virus entry and provides additional evidence that fully human antibodies developed from transgenic mice maintain neutralization capacity after conversion of the Fc region.

To demonstrate that the fully human monoclonal antibodies limit virus proliferation in an in vivo model, fibroblasts infected with TB40/E (moi: 0.01, 500,000 cells and ˜10% infected cells) were embedded in gelfoam. The gelfoam was implanted subcutaneously in three mice per group. The animals were treated with (1) isotype control, (2) anti-gH mAb clones 9A12, 13G1, and 15G11, each at 4 mg/kg or 18 mg/kg, respectively, or (3) the antiviral ganciclovir (GCV) at 50 mg/kg, respectively. Treatments were administrated every three days for nine days. At day ten post-implant, the gelfoam was removed and cells were released from the gel foam by collagenase treatment. The total DNA was harvest from the cells, quantified, and subjected to qPCR for UL123 (CMV gene) and b-actin (housekeeping geen) using SYBR green to evaluate virus proliferation. The qPCR analysis was performed in triplicate from three animals to determine the Ct values for UL123 and b-actin to calculate the relative levels of CMV DNA from the gelfoam samples.

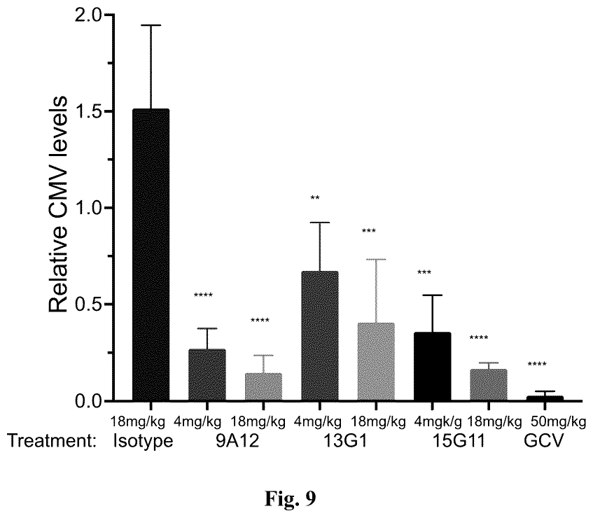

The mAb treatments significantly decreased CMV DNA levels in a concentration dependent manner (FIG. 9). The control ganciclovir was also effective at decreasing CMV DNA levels upon treatment. These data demonstrate that antibody treatment can limit virus proliferation in vivo. The antibodies are preventing viral dissemination by blocking cell-to-spread of virus.

### Overview of Sequences

An overview of nucleic acid sequences disclosed herein is shown in Table 8. An overview of amino acid sequences disclosed herein is shown in Tables 9 and 10.

TABLE 8
Nucleic acid sequences disclosed herein. n/a = not available.

| SEQ | | | |
|---|---|---|---|
| ID | | Anti- | |
| NO | Description | body | Sequence |
| 1 | CDR1H | 13G1 | gggttctcactctacaatgctagaatgggt |
| 2 | CDR2H | 13G1 | attttttcgaatgacgaaaga |
| 3 | CDR3H | 13G1 | gcacggatcgactacagtaactacataggaaactggttcgacccc |
| 4 | CDR1L | 13G1 | cagagtataagtaattac |
| n/a | CDR2L | 13G1 | gctgcatcc |
| 6 | CDR3L | 13G1 | caacagagttacagtaccccgttcact |
| 7 | VH | 13G1 | caggtcaccttgaaggagtctggtcctgtgctggtgaaacccacagagaccctcacgc |
| | | | tgacctgcaccgtctctgggttctcactctacaatgctagaatgggtgtgacctggatcc |
| | | | gtcagcccccagggaaggccctggagtggcttgcacacattttttcgaatgacgaaag |
| | | | atcctacacctcatctctgaagaacagactcaccatctccaaggacacctccaaaagcc |
| | | | aggtggtccttaccatgaccaacatggaccctgtggacacagccacatattactgtgca |
| | | | cggatcgactacagtaactacataggaaactggttcgacccctggggccagggaacc |
| | | | ctggtcaccgtctcctca |
| 8 | VL | 13G1 | gacatccagatgacccagtctccatcctccctgtctgcatctgtaggagacagagtcac |
| | | | catcacttgccgggcaagtcagagtataagtaattacttaaattggtatcagcagaaacc |
| | | | agggaaagcccctaagctcctgatctatgctgcatccagtttgcaaggtggggtcccat |
| | | | caaggttcagtggcagtggatctgggacagatttcactctcaccatcagcagtctgcaa |
| | | | cctgaagattttacaacttactactgtcaacagagttacagtaccccgttcactttcggcg |
| | | | gagggaccaaggtggagatcaaa |
| 9 | CDR1H | 14E1 | ggttacacctttaccagctatagt |
| 10 | CDR2H | 14E1 | atcaacacttacaatggtaacaca |
| 11 | CDR3H | 14E1 | gcgagagatcatgggttcttctttgactac |
| 12 | CDR1L | 14E1 | cagggcattagcagttat |
| n/a | CDR2L | 14E1 | gctgcatcc |
| 14 | CDR3L | 14E1 | caacagcttaatagttacccgtacact |
| 15 | VH | 14E1 | caggttcagctggtgcagtctggagctgaggtgaagaagcctggggcctcagtgaag |
| | | | gtctcctgcaaggcttctggttacacctttaccagctatagtatcagctgggtgcgacag |
| | | | gcccctggacaagggcttgagtggatgggatggatcaacacttacaatggtaacacaa |
| | | | actatgcacagagggtccaggccagagtcaccatgaccacagacacatccacgagca |
| | | | cagcctacatggagctgaggagcctgagatctgacgacacggccgtgtattactgtgc |
| | | | gagagatcatgggttcttctttgactactggggccagggaaacctggtcaccgtctcctc |
| | | | a |
| 16 | VL | 14E1 | gacatccagttgacccagtctccatccttcctgtctgcatctgtaggagacagagtcacc |
| | | | atcacttgctgggccagtcagggcattagcagttatttagcctggtatcagcaaaaacca |
| | | | gggaaagcccctaagctcctgatctatgctgcatccactttgcaaagtggggtcccatc |
| | | | aaggttcagcggcagtggatctgggacagaattcactctcacaatcagcagcctgcag |
| | | | cctgaagattttgcaacttattactgtcaacagcttaatagttacccgtacacttttggccag |
| | | | gggacccagctggagatcaaa |
| 17 | CDR1H | 15G11 | ggtggctccatcagcagtggttattactac |
| 18 | CDR2H | 15G11 | atctattacagtgggaccacc |
| 19 | CDR3H | 15G11 | gcgagagggggactgggggcttttgatatc |
| 20 | CDR1L | 15G11 | cagagtgttagcagcagctac |
| n/a | CDR2L | 15G11 | ggtgcatcc |
| 22 | CDR3L | 15G11 | cagcagtatggtatctcaccgctcact |
| 23 | VH | 15G11 | caggtgcagctgcaggagtcgggcccaggactggtgaagccttcacagaccctgtcc |
| | | | ctcacctgcactgtctctggtggctccatcagcagtggttattactactggaactggatcc |
| | | | gccagcacccagggaagggcctggaatgtattgggtacatctattacagtgggaccac |
| | | | ctactacaacccgtccctcaagagtcgacttttcatatcagtagacacgtctaagaacca |
| | | | gttctccctgaagctgagatctgtgactgccgcggacacggccgtgtattactgtgcga |
| | | | gagggggactgggggcttttgatatctggggccaagggacaatggtcaccgtctcttc |
| | | | a |
| 24 | VL | 15G11 | gaaattgtgttgacgcagtctccaggcaccctgtctttgtctccaggggaaagagccac |
| | | | cctctcctgcagggccagtcagagtgttagcagcagctacttagcctggtaccagcag |
| | | | aaacctggccaggctcccaggctcctcatctatggtgcatccagcagggcccctggca |
| | | | tcccagacaggttcagtggcactgggtctgggacagacttcactctcaccatcagcaga |
| | | | ctggagcctgaagattttgcagtgtattactgtcagcagtatggtatctcaccgctcacttt |
| | | | cggcggagggaccaaggtggagatcaaa |
| 25 | CDR1H | 9A12 | ggattcaccttcagtagtcatgaa |
| 26 | CDR2H | 9A12 | attagtagtactggtagtacaata |
| 27 | CDR3H | 9A12 | gcgaggagcagcggctggtatcggaactactcctatggtatggacgtc |
| 28 | CDR1L | 9A12 | cagggcatttacaattat |
| n/a | CDR2L | 9A12 | gctgcatcc |
| 30 | CDR3L | 9A12 | caacagtataatagttttcctcggacg |
| 31 | VH | 9A12 | gaggtgcagctggtggagtctgggggagtcttggttcagcctggagggtccctgagac |
| | | | tctcctgtgcagcctctggattcaccttcagtagtcatgaaatgaactgggtccgccagg |
| | | | ctccaggaaaggggctggagtggatttcatatattagtagtactggtagtacaatatacg |
| | | | acgcagactctgtgaagggccgattcaccatttccagagacaacgccaagaattcattg |
| | | | tatctgcaaatgaacagcctgagagccgaggacacggctgtttattactgtgcgaggag |
| | | | cagcggctggtatcggaactactcctatggtatggacgtctggggccaggggaccac |
| | | | ggtcaccgtctcctca |
| 32 | VL | 9A12 | gacatccagatggcccagtctccatcctcactgtctgcttcagtgggagacagagtcac |
| | | | catcacttgtcgggcgagtcagggcatttacaattatttagcctggtttcagcagaaacca |
| | | | gggaaagcccctaagtccctgatctatgctgcatccagtttgcaaactggggtcccctc |
| | | | aaagttcagcggcagtggatctgggacagatttcactctcaccatcagcaacctgcagc |
| | | | ctgaagattttgcaacttattactgccaacagtataatagttttcctcggacgttcggccaa |
| | | | gggaccaaggtggaaatcaaa |

TABLE 9
Amino acid sequences of selected antibodies disclosed herein

| SEQ | | | |
|---|---|---|---|
| ID | | Anti- | |
| NO | Description | body | Sequence |
| 33 | CDR1H | 13G1 | GFSLYNARMG |
| 34 | CDR2H | 13G1 | IFSNDER |
| 35 | CDR3H | 13G1 | ARIDYSNYIGNWFDP |
| 36 | CDR1L | 13G1 | QSISNY |
| n/a | CDR2L | 13G1 | AAS |
| 37 | CDR3L | 13G1 | QQSYSTPFT |
| 38 | VH | 13G1 | QVTLKESGPVLVKPTETLTLTCTVSGFSLYNARMGVT |
| | | | WIRQPPGKALEWLAHIFSNDERSYTSSLKNRLTISKDT |
| | | | SKSQVVLTMTNMDPVDTATYYCARIDYSNYIGNWFD |
| | | | PWGQGTLVTVSS |
| 39 | VL | 13G1 | DIQMTQSPSSLSASVGDRVTITCRASQSISNYLNWYQ |
| | | | QKPGKAPKLLIYAASSLQGGVPSRFSGSGSGTDFTLTI |
| | | | SSLQPEDFTTYYCQQSYSTPFTFGGGTKVEIK |
| 40 | CDR1H | 14E1 | GYTFTSYS |
| 41 | CDR2H | 14E1 | INTYNGNT |
| 42 | CDR3H | 14E1 | ARDHGFFFDY |
| 43 | CDR1L | 14E1 | QGISSY |
| n/a | CDR2L | 14E1 | AAS |
| 44 | CDR3L | 14E1 | QQLNSYPYT |
| 45 | VH | 14E1 | QVQLVQSGAEVKKPGASVKVSCKASGYTFTSYSISW |
| | | | VRQAPGQGLEWMGWINTYNGNTNYAQRVQARVTM |
| | | | TTDTSTSTAYMELRSLRSDDTAVYYCARDHGFFFDY |
| | | | WGQGNLVTVSS |
| 46 | VL | 14E1 | DIQLTQSPSFLSASVGDRVTITCWASQGISSYLAWYQ |
| | | | QKPGKAPKLLIYAASTLQSGVPSRFSGSGSGTEFTLTI |
| | | | SSLQPEDFATYYCQQLNSYPYTFGQGTQLEIK |
| 47 | CDR1H | 15G11 | GGSISSGYYY |
| 48 | CDR2H | 15G11 | IYYSGTT |
| 49 | CDR3H | 15G11 | ARGGLGAFDI |
| 50 | CDR1L | 15G11 | QSVSSSY |
| n/a | CDR2L | 15G11 | GAS |
| 51 | CDR3L | 15G11 | QQYGISPLT |
| 52 | VH | 15G11 | QVQLQESGPGLVKPSQTLSLTCTVSGGSISSGYYYWN |
| | | | WIRQHPGKGLECIGYIYYSGTTYYNPSLKSRLFISVDT |
| | | | SKNQFSLKLRSVTAADTAVYYCARGGLGAFDIWGQG |
| | | | TMVTVSS |
| 53 | VL | 15G11 | EIVLTQSPGTLSLSPGERATLSCRASQSVSSSYLAWYQ |
| | | | QKPGQAPRLLIYGASSRAPGIPDRFSGTGSGTDFTLTIS |
| | | | RLEPEDFAVYYCQQYGISPLTFGGGTKVEIK |
| 54 | CDR1H | 9A12 | GFTFSSHE |
| 55 | CDR2H | 9A12 | ISSTGSTI |
| 56 | CDR3H | 9A12 | ARSSGWYRNYSYGMDV |
| 57 | CDR1L | 9A12 | QGIYNY |
| n/a | CDR2L | 9A12 | AAS |
| 58 | CDR3L | 9A12 | QQYNSFPRT |
| 59 | VH | 9A12 | EVQLVESGGVLVQPGGSLRLSCAASGFTFSSHEMNW |
| | | | VRQAPGKGLEWISYISSTGSTIYDADSVKGRFTISRDN |
| | | | AKNSLYLQMNSLRAEDTAVYYCARSSGWYRNYSYG |
| | | | MDVWGQGTTVTVSS |
| 60 | VL | 9A12 | DIQMAQSPSSLSASVGDRVTITCRASQGIYNYLAWFQ |
| | | | QKPGKAPKSLIYAASSLQTGVPSKFSGSGSGTDFTLTI |
| | | | SNLQPEDFATYYCQQYNSFPRTFGQGTKVEIK |

TABLE 10
Additional amino acid sequences disclosed herein

| SEQ | | |
|---|---|---|
| ID | | |
| NO | Description | Sequence |
| 61 | Peptide derived | PAHSRYGPQAVDAR |
| | from the CMV | |
| | TB40/E gL | |
| | sequence | |
| 62 | Linearized | KRKMDPDNPDEGPS |
| | peptide | |
| 63 | gH | MRPGLPFYLTVFAVYLLSHLPSQRYGADAASEALDPHAF |
| | | HLLLNTYGRPIRFLRENTTQCTYNSSLRNSTVVRENAISF |
| | | NFFQSYNQYYVFHMPRCLFAGPLAEQFLNQVDLTETLE |
| | | RYQQRLNTYALVSKDLASYRSFSQQLKAQDSLGQQPTT |
| | | VPPPIDLSIPHVWMPPQTTPHDWKGSHTTSGLHRPHENQ |
| | | TCILFDGHDLLFSTVTPCLHQGFYLMDELRYVKITLTEDF |
| | | FVVTVSIDDDTPMLLIFGHLPRVLFKAPYQRDNFILRQTE |
| | | KHELLVLVKKTQLNRHSYLKDSDFLDAALDFNYLDLSA |
| | | LLRNSFHRYAVDVLKSGRCQMLDRRTVEMAFAYALAL |
| | | FAAARQEEAGTEISIPRALDRQAALLQIQEFMITCLSQTPP |
| | | RTTLLLYPTAVDLAKRALWTPDQITDITSLVRLVYILSKQ |
| | | NQQHLIPQWALRQIADFALQLHKTHLASFLSAFARQELY |
| | | LMGSLVHSMLVHTTERREIFIVETGLCSLAELSHFTQLLA |
| | | HPHHEYLSDLYTPCSSSGRRDHSLERLTRLFPDATVPAT |
| | | VPAALSILSTMQPSTLETFPDLFCLPLGESFSALTVSEHVS |
| | | YVVTNQYLIKGISYPVSTTVVGQSLIITQTDSQSKCELTR |
| | | NMHTTHSITAALNISLENCAFCQSALLEYDDTQGVINIM |
| | | YMHDSDDVLFALDPYNEVVVSSPRTHYLMLLKNGTVLE |
| | | VTDVVVDATDSRLLMMSVYALSAIIGIYLLYRMLKTC |
| 64 | gL | MCRRPDCGFSFSPGPVVLLWCCLLLPIVSSVAVSVAPTA |
| | | AEKVPAECPELTRRCLLGEVFQGDKYESWLRPLVNVTR |
| | | RDGPLSQLIRYRPVTPEAANSVLLDDAFLDTLALLYNNP |
| | | DQLRALLTLLSSDTAPRWMTVMRGYSECGDGSPAVYTC |
| | | VDDLCRGYDLTRLSYGRSIFTEVLGFELVPPSLFNVVVAI |
| | | RNEATRTNRAVRLPVSTAAAPEGITLFYGLYNAVKEFCL |
| | | RHQLDPPLLRHLDKYYAGLPPELKQTRVNLPAHSRYGP |
| | | QAVDAR |
| 65 | Linker | GSGSGSG |
| 66 | Control peptide | YPYDVPDYAG |
| 67 | Highly | HTTERREIFI |
| | conserved region | |
| | of gH | |

We claim:

1. An antibody or antigen-binding fragment thereof which binds to human cytomelagovirus (HCMV), the antibody or antigen-binding fragment thereof comprising a heavy chain variable region and a light chain variable region, wherein each of the heavy chain and the light chain variable regions comprises a CDR1, CDR2, and CDR3, and wherein:
(a) the sequence of CDR1H comprises SEQ ID NO:33;
the sequence of CDR2H comprises SEQ ID NO:34;
the sequence of CDR3H comprises SEQ ID NO:35;
the sequence of CDR1L comprises SEQ ID NO:36;
the sequence of CDR2L comprises sequence AAS; and
the sequence of CDR3L comprises SEQ ID NO:37;
(b) the sequence of CDR1H comprises SEQ ID NO:40;
the sequence of CDR2H comprises SEQ ID NO:41;
the sequence of CDR3H comprises SEQ ID NO:42;
the sequence of CDR1L comprises SEQ ID NO:43;
the sequence of CDR2L comprises sequence AAS; and
the sequence of CDR3L comprises SEQ ID NO:44;
(c) the sequence of CDR1H comprises SEQ ID NO:47;
the sequence of CDR2H comprises SEQ ID NO:48;
the sequence of CDR3H comprises SEQ ID NO:49;
the sequence of CDR1L comprises SEQ ID NO:50;
the sequence of CDR2L comprises sequence GAS; and
the sequence of CDR3L comprises SEQ ID NO:51; or
(d) the sequence of CDR1H comprises SEQ ID NO:54;
the sequence of CDR2H comprises SEQ ID NO:55;
the sequence of CDR3H comprises SEQ ID NO:56;
the sequence of CDR1L comprises SEQ ID NO:57;
the sequence of CDR2L comprises sequence AAS; and
the sequence of CDR3L comprises SEQ ID NO:58.

2. The antibody or antigen-binding fragment thereof of claim 1, wherein:
(a) the sequence of the heavy chain variable region comprises a sequence that is at least 90% identical to SEQ ID NO:38 and wherein the sequence of the light chain variable region comprises a sequence that is at least 90% identical to SEQ ID NO:39;
(b) the sequence of the heavy chain variable region comprises a sequence that is at least 90% identical to SEQ ID NO:45 and wherein the sequence of the light chain variable region comprises a sequence that is at least 90% identical to SEQ ID NO:46;
(c) the sequence of the heavy chain variable region comprises a sequence that is at least 90% identical to SEQ ID NO:52 and wherein the sequence of the light chain variable region comprises a sequence that is at least 90% identical to SEQ ID NO:53; or
(d) the sequence of the heavy chain variable region comprises a sequence that is at least 90% identical to SEQ ID NO:59 and wherein the sequence of the light chain variable region comprises a sequence that is at least 90% identical to SEQ ID NO:60.

3. The antibody or antigen-binding fragment thereof of claim 2, wherein:
(a) the sequence of the heavy chain variable region comprises a sequence that is at least 95% identical to SEQ ID NO:38 and wherein the sequence of the light chain variable region comprises a sequence that is at least 95% identical to SEQ ID NO:39;
(b) the sequence of the heavy chain variable region comprises a sequence that is at least 95% identical to SEQ ID NO:45 and wherein the sequence of the light chain variable region comprises a sequence that is at least 95% identical to SEQ ID NO:46;
(c) the sequence of the heavy chain variable region comprises a sequence that is at least 95% identical to SEQ ID NO:52 and wherein the sequence of the light chain variable region comprises a sequence that is at least 95% identical to SEQ ID NO:53; or
(d) the sequence of the heavy chain variable region comprises a sequence that is at least 95% identical to SEQ ID NO:59 and wherein the sequence of the light chain variable region comprises a sequence that is at least 95% identical to SEQ ID NO:60.

4. The antibody or antigen-binding fragment thereof according claim 3, wherein:
(a) the sequence of the heavy chain variable region comprises SEQ ID NO:38 and the sequence of the light chain variable region comprises SEQ ID NO:39;
(b) the sequence of the heavy chain variable region comprises SEQ ID NO:45 and the sequence of the light chain variable region comprises SEQ ID NO:46;
(c) the sequence of the heavy chain variable region comprises SEQ ID NO:52 and the sequence of the light chain variable region comprises SEQ ID NO:53; or
(d) the sequence of the heavy chain variable region comprises SEQ ID NO:59 and the sequence of the light chain variable region comprises SEQ ID NO:60.

5. The antibody or antigen-binding fragment thereof of any one of claims 1-3, wherein the antibody or antigen-binding fragment thereof is a chimeric antibody, a CDR-grafted antibody, or a humanized antibody or antigen-binding fragment thereof.

6. The antibody or antigen-binding fragment thereof according to any of the preceding claims, wherein the antibody or antigen-binding fragment thereof is a monoclonal antibody or antigen-binding fragment thereof.

7. The antibody or antigen-binding fragment thereof according to any of the preceding claims, wherein the antibody or antigen-binding fragment thereof is a multispecific or a bispecific antibody or antigen-binding fragment thereof.

8. The antibody or antigen-binding fragment thereof according to any of the preceding claims, wherein the antibody or antigen-binding fragment thereof is an scFv, Fv, Fab′, Fab, F(ab′)₂, or diabody.

9. The antibody or antigen-binding fragment thereof according to any of the preceding claims, wherein the antibody or antigen-binding fragment thereof has isotype IgG2.

10. The antibody or antigen-binding fragment thereof according to any of the preceding claims, wherein the antibody or antigen-binding portion thereof is capable of broadly neutralizing an HCMV infection.

11. An isolated nucleic acid encoding the antibody or antigen-binding fragment thereof according to any of the preceding claims.

12. A vector comprising the nucleic acid of claim 11.

13. A vector or set of vectors encoding an antibody or antigen-binding fragment thereof which binds to HCMV, the antibody or antigen-binding fragment thereof comprising a heavy chain variable region and a light chain variable region, wherein:
(a) the sequence encoding the heavy chain variable region comprises a sequence that is at least 80% identical to SEQ ID NO:7 and the sequence encoding the light chain variable region comprises a sequence that is at least 80% identical to SEQ ID NO:8;
(b) the sequence encoding the heavy chain variable region comprises a sequence that is at least 80% identical to SEQ ID NO:15 and the sequence encoding the light chain variable region comprises a sequence that is at least 80% identical to SEQ ID NO:16;
(c) the sequence encoding the heavy chain variable region comprises a sequence that is at least 80% identical to SEQ ID NO:23 and the sequence encoding the light chain variable region comprises a sequence that is at least 80% identical to SEQ ID NO:24; or
(d) the sequence encoding the heavy chain variable region comprises a sequence that is at least 80% identical to SEQ ID NO:31 and the sequence encoding the light chain variable region comprises a sequence that is at least 80% identical to SEQ ID NO:32.

14. The vector or set of vectors of claim 13, wherein:
(a) the sequence encoding the heavy chain variable region comprises SEQ ID NO:7 and the sequence encoding the light chain variable region comprises SEQ ID NO:8;
(b) the sequence encoding the heavy chain variable region comprises SEQ ID NO:15 and the sequence encoding the light chain variable region comprises SEQ ID NO:16;
(c) the sequence encoding the heavy chain variable region comprises SEQ ID NO:23 and the sequence encoding the light chain variable region comprises SEQ ID NO:24; or
(d) the sequence encoding the heavy chain variable region comprises SEQ ID NO:31 and the sequence encoding the light chain variable region comprises SEQ ID NO:32.

15. A cell comprising the vector of claim 12 or the vector or set of vectors of any one of claims 13-14.

16. The cell of claim 15, wherein the cell is a bacterial cell, a yeast cell, or an isolated mammalian cell.

17. A pharmaceutical composition comprising the antibody or antigen-binding fragment thereof of any one of claims 1-10 and a pharmaceutically acceptable carrier or excipient.

18. A T-cell comprising a chimeric antigen receptor comprising the CDRs of the antibody or antigen-binding fragment thereof of any one of claims 1-10.

19. The antibody or antigen-binding fragment thereof of any one of claims 1-10, wherein the antibody or antigen-binding fragment thereof is conjugated to one or more of a cytotoxin, a fluorescent label, and an imaging agent.

20. A kit for detecting the presence of HCMV, or an antigenic fragment of HCMV thereof, in a sample comprising: (i) the antibody or antigen-binding portion thereof of any one of claims 1-10, and (ii) a buffer.

21. The kit of claim 20, wherein the antibody or antigen-binding portion thereof is bound to a substrate.

22. The kit of any one of claims 20-21, wherein the antibody or antigen-binding portion thereof is detectably labeled.

23. The kit of any one of claims 20-22, the kit further comprising a secondary antibody that specifically binds to the antibody or antigen-binding portion thereof.

24. The kit of claim 23, wherein the secondary antibody is an anti-IgG antibody.

25. The kit of any one of claims 23-24, wherein the secondary antibody is detectably labeled.

26. A method of making an antibody or antigen-binding fragment thereof which binds to HCMV, the method comprising:
(i) providing a cell comprising one or more nucleic acid molecules encoding the antibody or antigen-binding fragment thereof of any one of claims 1-10;
(ii) expressing in the cell at least one of a heavy variable chain, a light variable chain, or combinations thereof; and
(iii) collecting the antibody or antigen-binding fragment thereof.

27. A method of detecting the presence of HCMV, or an antigenic fragment thereof, in a sample comprising:
(i) obtaining a sample containing, or suspecting of containing, HCMV, or an antigenic fragment thereof;
(ii) contacting the sample with the antibody or antigen-binding fragment thereof of any one of claims 1-10; and
(iii) detecting the presence of specific binding of the antibody or antigen-binding fragment thereof to HCMV, or an antigenic fragment thereof.

28. The method of claim 27, further comprising quantifying the amount of HCMV, or antigenic fragments thereof, present in the sample.

29. The method of any one of claims 27-28, wherein the sample is an environmental sample.

30. The method of any one of claims 27-28, wherein the sample is a biological sample.

31. A method of treating an HCMV infection in a subject in need thereof comprising, the method comprising administering to the subject the antibody or antigen-binding fragment thereof of any one of claims 1-10.

32. The method of claim 31, the method further comprising administering to the individual at least one additional anti-HCMV antibody or antigen-binding portion thereof.

33. The method of claim 32, wherein the at least one additional anti-HCMV antibody, or antigen-binding portion thereof, is an antibody or antigen-binding fragment thereof of any one of claims 1-10.

34. The method of any one of claims 31-33, further comprising administering to the individual at least one additional antiviral composition.

35. The method of claim 34, wherein the at least one additional antiviral composition is selected from the group consisting of ganciclovir, valganciclovir, foscarnet, cidofovir, and combinations thereof.

36. A method of preventing an HCMV infection in a subject comprising administering to the individual the antibody or antigen-binding fragment thereof of any one of claims 1-10.

37. A method of diagnosing a subject as having an HCMV infection comprising:
(i) identifying a subject;
(ii) obtaining from the subject a biological sample containing HCMV or an antigenic fragment thereof;
(iii) contacting the sample with the antibody or antigen-binding fragment thereof of any one of claims 1-10;
(iv) detecting the presence of specific binding of the antibody or antigen-binding fragment thereof to HCMV, or an antigenic fragment thereof; and
(v) diagnosing the subject as having an HCMV infection.

38. A method of inhibiting binding of HCMV glycoprotein gH and/or glycoprotein gL to a cellular surface protein, the method comprising contacting gH and/or gL with the antibody or antigen-binding fragment thereof of any one of claims 1-10.

39. The method of claim 38, wherein the cellular surface protein is selected from the group consisting of Nectin 1, EphA2, Nrp2, PDGFRalpha.

40. The antibody or antigen-binding fragment thereof of any one of claims 1-10 for use in medicine.

41. The antibody or antigen-binding fragment thereof of any one of claims 1-10 for use in treating or preventing an HCMV infection.

42. Use of the antibody or antigen-binding fragment thereof of any one of claims 1-10 in the manufacture of a medicament for use in treating or preventing an HCMV infection.


SEQUENCE LISTING (67 sequences)

SEQ ID NO: 1 - DNA, 30 bp, synthetic construct
  source 1..30: mol_type = other DNA
gggttctcactctacaatgctagaatgggt

SEQ ID NO: 2 - DNA, 21 bp, synthetic construct
  source 1..21: mol_type = other DNA
attttttcgaatgacgaaaga

SEQ ID NO: 3 - DNA, 45 bp, synthetic construct
  source 1..45: mol_type = other DNA
gcacggatcgactacagtaactacataggaaactggttcgacccc

SEQ ID NO: 4 - DNA, 18 bp, synthetic construct
  source 1..18: mol_type = other DNA
cagagtataagtaattac

SEQ ID NO: 5 - skipped

SEQ ID NO: 6 - DNA, 27 bp, synthetic construct
  source 1..27: mol_type = other DNA
caacagagttacagtaccccgttcact

SEQ ID NO: 7 - DNA, 369 bp, synthetic construct
  source 1..369: mol_type = other DNA
caggtcaccttgaaggagtctggtcctgtgctggtgaaacccacagagaccctcacgctgacctgcaccgtctctgggttctcactctacaatgctagaatgggtgtgacctggatccgtcagcccccagggaaggccctggagtggcttgcacacattttttcgaatgacgaaagatcctacacctcatctctgaagaacagactcaccatctccaaggacacctccaaaagccaggtggtccttaccatgaccaacatggaccctgtggacacagccacatattactgtgcacggatcgactacagtaactacataggaaactggttcgacccctggggccagggaaccctggtcaccgtctcctca

SEQ ID NO: 8 - DNA, 321 bp, synthetic construct
  source 1..321: mol_type = other DNA
gacatccagatgacccagtctccatcctccctgtctgcatctgtaggagacagagtcaccatcacttgccgggcaagtcagagtataagtaattacttaaattggtatcagcagaaaccagggaaagcccctaagctcctgatctatgctgcatccagtttgcaaggtggggtcccatcaaggttcagtggcagtggatctgggacagatttcactctcaccatcagcagtctgcaacctgaagattttacaacttactactgtcaacagagttacagtaccccgttcactttcggcggagggaccaaggtggagatcaaa

SEQ ID NO: 9 - DNA, 24 bp, synthetic construct
  source 1..24: mol_type = other DNA
ggttacacctttaccagctatagt

SEQ ID NO: 10 - DNA, 24 bp, synthetic construct
  source 1..24: mol_type = other DNA
atcaacacttacaatggtaacaca

SEQ ID NO: 11 - DNA, 30 bp, synthetic construct
  source 1..30: mol_type = other DNA
gcgagagatcatgggttcttctttgactac

SEQ ID NO: 12 - DNA, 18 bp, synthetic construct
  source 1..18: mol_type = other DNA
cagggcattagcagttat

SEQ ID NO: 13 - skipped

SEQ ID NO: 14 - DNA, 27 bp, synthetic construct
  source 1..27: mol_type = other DNA
caacagcttaatagttacccgtacact

SEQ ID NO: 15 - DNA, 351 bp, synthetic construct
  source 1..351: mol_type = other DNA
caggttcagctggtgcagtctggagctgaggtgaagaagcctggggcctcagtgaaggtctcctgcaaggcttctggttacacctttaccagctatagtatcagctgggtgcgacaggcccctggacaagggcttgagtggatgggatggatcaacacttacaatggtaacacaaactatgcacagagggtccaggccagagtcaccatgaccacagacacatccacgagcacagcctacatggagctgaggagcctgagatctgacgacacggccgtgtattactgtgcgagagatcatgggttcttctttgactactggggccagggaaacctggtcaccgtctcctca

SEQ ID NO: 16 - DNA, 321 bp, synthetic construct
  source 1..321: mol_type = other DNA
gacatccagttgacccagtctccatccttcctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttacccgtacacttttggccaggggacccagctggagatcaaa

SEQ ID NO: 17 - DNA, 30 bp, synthetic construct
  source 1..30: mol_type = other DNA
ggtggctccatcagcagtggttattactac

SEQ ID NO: 18 - DNA, 21 bp, synthetic construct
  source 1..21: mol_type = other DNA
atctattacagtgggaccacc

SEQ ID NO: 19 - DNA, 30 bp, synthetic construct
  source 1..30: mol_type = other DNA
gcgagagggggactgggggcttttgatatc

SEQ ID NO: 20 - DNA, 21 bp, synthetic construct
  source 1..21: mol_type = other DNA
cagagtgttagcagcagctac

SEQ ID NO: 21 - skipped

SEQ ID NO: 22 - DNA, 27 bp, synthetic construct
  source 1..27: mol_type = other DNA
cagcagtatggtatctcaccgctcact

SEQ ID NO: 23 - DNA, 354 bp, synthetic construct
  source 1..354: mol_type = other DNA
caggtgcagctgcaggagtcgggcccaggactggtgaagccttcacagaccctgtccctcacctgcactgtctctggtggctccatcagcagtggttattactactggaactggatccgccagcacccagggaagggcctggaatgtattgggtacatctattacagtgggaccacctactacaacccgtccctcaagagtcgacttttcatatcagtagacacgtctaagaaccagttctccctgaagctgagatctgtgactgccgcggacacggccgtgtattactgtgcgagagggggactgggggcttttgatatctggggccaagggacaatggtcaccgtctcttca

SEQ ID NO: 24 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggcccctggcatcccagacaggttcagtggcactgggtctgggacagacttcactctcaccatcagcagactggagcctgaagattttgcagtgtattactgtcagcagtatggtatctcaccgctcactttcggcggagggaccaaggtggagatcaaa

SEQ ID NO: 25 - DNA, 24 bp, synthetic construct
  source 1..24: mol_type = other DNA
ggattcaccttcagtagtcatgaa

SEQ ID NO: 26 - DNA, 24 bp, synthetic construct
  source 1..24: mol_type = other DNA
attagtagtactggtagtacaata

SEQ ID NO: 27 - DNA, 48 bp, synthetic construct
  source 1..48: mol_type = other DNA
gcgaggagcagcggctggtatcggaactactcctatggtatggacgtc

SEQ ID NO: 28 - DNA, 18 bp, synthetic construct
  source 1..18: mol_type = other DNA
cagggcatttacaattat

SEQ ID NO: 29 - skipped

SEQ ID NO: 30 - DNA, 27 bp, synthetic construct
  source 1..27: mol_type = other DNA
caacagtataatagttttcctcggacg

SEQ ID NO: 31 - DNA, 369 bp, synthetic construct
  source 1..369: mol_type = other DNA
gaggtgcagctggtggagtctgggggagtcttggttcagcctggagggtccctgagactctcctgtgcagcctctggattcaccttcagtagtcatgaaatgaactgggtccgccaggctccaggaaaggggctggagtggatttcatatattagtagtactggtagtacaatatacgacgcagactctgtgaagggccgattcaccatttccagagacaacgccaagaattcattgtatctgcaaatgaacagcctgagagccgaggacacggctgtttattactgtgcgaggagcagcggctggtatcggaactactcctatggtatggacgtctggggccaggggaccacggtcaccgtctcctca

SEQ ID NO: 32 - DNA, 321 bp, synthetic construct
  source 1..321: mol_type = other DNA
gacatccagatggcccagtctccatcctcactgtctgcttcagtgggagacagagtcaccatcacttgtcgggcgagtcagggcatttacaattatttagcctggtttcagcagaaaccagggaaagcccctaagtccctgatctatgctgcatccagtttgcaaactggggtcccctcaaagttcagcggcagtggatctgggacagatttcactctcaccatcagcaacctgcagcctgaagattttgcaacttattactgccaacagtataatagttttcctcggacgttcggccaagggaccaaggtggaaatcaaa

SEQ ID NO: 33 - protein, 10 aa, synthetic construct
GFSLYNARMG

SEQ ID NO: 34 - protein, 7 aa, synthetic construct
IFSNDER

SEQ ID NO: 35 - protein, 15 aa, synthetic construct
ARIDYSNYIGNWFDP

SEQ ID NO: 36 - protein, 6 aa, synthetic construct
QSISNY

SEQ ID NO: 37 - protein, 9 aa, synthetic construct
QQSYSTPFT

SEQ ID NO: 38 - protein, 123 aa, synthetic construct
QVTLKESGPVLVKPTETLTLTCTVSGFSLYNARMGVTWIRQPPGKALEWLAHIFSNDERSYTSSLKNRLTISKDTSKSQVVLTMTNMDPVDTATYYCARIDYSNYIGNWFDPWGQGTLVTVSS

SEQ ID NO: 39 - protein, 107 aa, synthetic construct
DIQMTQSPSSLSASVGDRVTITCRASQSISNYLNWYQQKPGKAPKLLIYAASSLQGGVPSRFSGSGSGTDFTLTISSLQPEDFTTYYCQQSYSTPFTFGGGTKVEIK

SEQ ID NO: 40 - protein, 8 aa, synthetic construct
GYTFTSYS

SEQ ID NO: 41 - protein, 8 aa, synthetic construct
INTYNGNT

SEQ ID NO: 42 - protein, 10 aa, synthetic construct
ARDHGFFFDY

SEQ ID NO: 43 - protein, 6 aa, synthetic construct
QGISSY

SEQ ID NO: 44 - protein, 9 aa, synthetic construct
QQLNSYPYT

SEQ ID NO: 45 - protein, 117 aa, synthetic construct
QVQLVQSGAEVKKPGASVKVSCKASGYTFTSYSISWVRQAPGQGLEWMGWINTYNGNTNYAQRVQARVTMTTDTSTSTAYMELRSLRSDDTAVYYCARDHGFFFDYWGQGNLVTVSS

SEQ ID NO: 46 - protein, 107 aa, synthetic construct
DIQLTQSPSFLSASVGDRVTITCWASQGISSYLAWYQQKPGKAPKLLIYAASTLQSGVPSRFSGSGSGTEFTLTISSLQPEDFATYYCQQLNSYPYTFGQGTQLEIK

SEQ ID NO: 47 - protein, 10 aa, synthetic construct
GGSISSGYYY

SEQ ID NO: 48 - protein, 7 aa, synthetic construct
IYYSGTT

SEQ ID NO: 49 - protein, 10 aa, synthetic construct
ARGGLGAFDI

SEQ ID NO: 50 - protein, 7 aa, synthetic construct
QSVSSSY

SEQ ID NO: 51 - protein, 9 aa, synthetic construct
QQYGISPLT

SEQ ID NO: 52 - protein, 118 aa, synthetic construct
QVQLQESGPGLVKPSQTLSLTCTVSGGSISSGYYYWNWIRQHPGKGLECIGYIYYSGTTYYNPSLKSRLFISVDTSKNQFSLKLRSVTAADTAVYYCARGGLGAFDIWGQGTMVTVSS

SEQ ID NO: 53 - protein, 108 aa, synthetic construct
EIVLTQSPGTLSLSPGERATLSCRASQSVSSSYLAWYQQKPGQAPRLLIYGASSRAPGIPDRFSGTGSGTDFTLTISRLEPEDFAVYYCQQYGISPLTFGGGTKVEIK

SEQ ID NO: 54 - protein, 8 aa, synthetic construct
GFTFSSHE

SEQ ID NO: 55 - protein, 8 aa, synthetic construct
ISSTGSTI

SEQ ID NO: 56 - protein, 16 aa, synthetic construct
ARSSGWYRNYSYGMDV

SEQ ID NO: 57 - protein, 6 aa, synthetic construct
QGIYNY

SEQ ID NO: 58 - protein, 9 aa, synthetic construct
QQYNSFPRT

SEQ ID NO: 59 - protein, 123 aa, synthetic construct
EVQLVESGGVLVQPGGSLRLSCAASGFTFSSHEMNWVRQAPGKGLEWISYISSTGSTIYDADSVKGRFTISRDNAKNSLYLQMNSLRAEDTAVYYCARSSGWYRNYSYGMDVWGQGTTVTVSS

SEQ ID NO: 60 - protein, 107 aa, synthetic construct
DIQMAQSPSSLSASVGDRVTITCRASQGIYNYLAWFQQKPGKAPKSLIYAASSLQTGVPSKFSGSGSGTDFTLTISNLQPEDFATYYCQQYNSFPRTFGQGTKVEIK

SEQ ID NO: 61 - protein, 14 aa, Human betaherpesvirus 5
PAHSRYGPQAVDAR

SEQ ID NO: 62 - protein, 14 aa, synthetic construct
KRKMDPDNPDEGPS

SEQ ID NO: 63 - protein, 743 aa, Human betaherpesvirus 5
MRPGLPFYLTVFAVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKTQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQSKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 64 - protein, 277 aa, Human betaherpesvirus 5
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 65 - protein, 7 aa, synthetic construct
GSGSGSG

SEQ ID NO: 66 - protein, 10 aa, synthetic construct
YPYDVPDYAG

SEQ ID NO: 67 - protein, 10 aa, Human betaherpesvirus 5
HTTERREIFI